SEQUENCE LISTING (86 sequences)

SEQ ID NO: 1 - protein, 20 aa, Mus musculus
MARSVTLVFLVLVSLTGLYA

SEQ ID NO: 2 - protein, 99 aa, Mus musculus
IQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDM

SEQ ID NO: 3 - protein, 275 aa, Mus musculus
GPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWE

SEQ ID NO: 4 - protein, 275 aa, Mus musculus
GPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQNMELVETRPAGDGTFQKWASVVVPLGKEQYYTCHVYHQGLPEPLTLRWE

SEQ ID NO: 5 - protein, 275 aa, Mus musculus
GPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELILNMELVETRPAGDGTFQKWASVVVPLGKEQYYTCHVYHQGLPEPLTLRWE

SEQ ID NO: 6 - protein, 8 aa, Gallus gallus
SIINFEKL

SEQ ID NO: 7 - protein, 8 aa, synthetic construct
  REGION 1..8: note = OVA-Y3-AA
SIYNFEKL

SEQ ID NO: 8 - protein, 8 aa, synthetic construct
  REGION 1..8: note = OVA-Q4-AA
SIIQFEKL

SEQ ID NO: 9 - protein, 8 aa, synthetic construct
  REGION 1..8: note = OVA-T4-AA
SIITFEKL

SEQ ID NO: 10 - protein, 8 aa, synthetic construct
  REGION 1..8: note = OVA-V4-AA
SIIVFEKL

SEQ ID NO: 11 - protein, 5 aa, synthetic construct
  REGION 1..5: note = linker-AA
GGGGS

SEQ ID NO: 12 - protein, 27 aa, Mus musculus
FWALVVVAGVLFCYGLLVTVALCVIWT

SEQ ID NO: 13 - protein, 27 aa, Homo sapiens
FWVLVVVGGVLACYSLLVTVAFIIFWV

SEQ ID NO: 14 - protein, 41 aa, Mus musculus
NSRRNRGGQSDYMNMTPRRPGLTRKPYQPYAPARDFAAYRP

SEQ ID NO: 15 - protein, 41 aa, Homo sapiens
RSKRSRGGHSDYMNMTPRRPGPTRKHYQPYAPPRDFAAYRS

SEQ ID NO: 16 - protein, 114 aa, Homo sapiens
DIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 113 aa, Mus musculus
RAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 19 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 20 - protein, 654 aa, synthetic construct
  REGION 1..654: note = OVA-bait-1-AA
MARSVTLVFLVLVSLTGLYASIINFEKLGGGGSGGGGSGGGGSIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 21 - DNA, 1974 bp, synthetic construct
  misc_feature 1..1974: note = OVA-bait-1-NT
  source 1..1974: mol_type = other DNA
atggccagatctgtgaccctggtgttcctggtgctggtgtctctgacaggactgtacgccagcatcatcaacttcgagaagctcggcggaggcggatctggtggcggaggaagtggcggcggaggatctatccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatgggaggcggtggaagcggaggtggtggatctggcggtggcggtagcggaggcggaggttctggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccaggacatggaactggtggaaaccagacctgctggcgacggcaccttccagaaatgggcttctgtggtggtgcccctgggcaaagagcagtactacacctgtcacgtgtaccaccagggcctgcctgaacctctgacactgagatgggaaggatccatcgagttcatgtaccctccaccttacctggacaacgagagaagcaacggcaccatcatccacatcaaagagaagcacctctgtcacacccagagcagccccaagctgttttgggctcttgtggtggtggctggcgtgctgttttgttacggcctgctcgtgacagtggccctgtgtgtgatctggaccaacagcagaagaaacagaggcggccagagcgactacatgaacatgacccctagaaggcctggcctgaccagaaagccttaccagccttacgctcccgccagagatttcgccgcttatagaccaccgcggagagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 22 - protein, 654 aa, synthetic construct
  REGION 1..654: note = OVA-bait-SIIQ-AA
MARSVTLVFLVLVSLTGLYASIIQFEKLGGGGSGGGGSGGGGSIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 23 - DNA, 1974 bp, synthetic construct
  misc_feature 1..1974: note = OVA-bait-SIIQ-NT
  source 1..1974: mol_type = other DNA
atggccagatctgtgaccctggtgttcctggtgctggtgtctctgacaggactgtacgccagcatcatccagttcgagaagctcggcggaggcggatctggtggcggaggaagtggcggcggaggatctatccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatgggaggcggtggaagcggaggtggtggatctggcggtggcggtagcggaggcggaggttctggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccaggacatggaactggtggaaaccagacctgctggcgacggcaccttccagaaatgggcttctgtggtggtgcccctgggcaaagagcagtactacacctgtcacgtgtaccaccagggcctgcctgaacctctgacactgagatgggaaggatccatcgagttcatgtaccctccaccttacctggacaacgagagaagcaacggcaccatcatccacatcaaagagaagcacctctgtcacacccagagcagccccaagctgttttgggctcttgtggtggtggctggcgtgctgttttgttacggcctgctcgtgacagtggccctgtgtgtgatctggaccaacagcagaagaaacagaggcggccagagcgactacatgaacatgacccctagaaggcctggcctgaccagaaagccttaccagccttacgctcccgccagagatttcgccgcttatagaccaccgcggagagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 24 - protein, 654 aa, synthetic construct
  REGION 1..654: note = OVA-bait-SIIT-AA
MARSVTLVFLVLVSLTGLYASIITFEKLGGGGSGGGGSGGGGSIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 25 - DNA, 1974 bp, synthetic construct
  misc_feature 1..1974: note = OVA-bait-SIIT-NT
  source 1..1974: mol_type = other DNA
atggccagatctgtgaccctggtgttcctggtgctggtgtctctgacaggactgtacgccagcatcatcaccttcgagaagctcggcggaggcggatctggtggcggaggaagtggcggcggaggatctatccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatgggaggcggtggaagcggaggtggtggatctggcggtggcggtagcggaggcggaggttctggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccaggacatggaactggtggaaaccagacctgctggcgacggcaccttccagaaatgggcttctgtggtggtgcccctgggcaaagagcagtactacacctgtcacgtgtaccaccagggcctgcctgaacctctgacactgagatgggaaggatccatcgagttcatgtaccctccaccttacctggacaacgagagaagcaacggcaccatcatccacatcaaagagaagcacctctgtcacacccagagcagccccaagctgttttgggctcttgtggtggtggctggcgtgctgttttgttacggcctgctcgtgacagtggccctgtgtgtgatctggaccaacagcagaagaaacagaggcggccagagcgactacatgaacatgacccctagaaggcctggcctgaccagaaagccttaccagccttacgctcccgccagagatttcgccgcttatagaccaccgcggagagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 26 - protein, 654 aa, synthetic construct
  REGION 1..654: note = OVA-bait-SIIV-AA
MARSVTLVFLVLVSLTGLYASIIVFEKLGGGGSGGGGSGGGGSIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQDMELVETRPAGDGTFQKWASVVVPLGKEQYYTCHVYHQGLPEPLTLRWEIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 27 - DNA, 1974 bp, synthetic construct
  misc_feature 1..1974: note = OVA-bait-SIIV-NT
  source 1..1974: mol_type = other DNA
atggccagatctgtgaccctggtgttcctggtgctggtgtctctgacaggactgtacgccagcatcatcgtgttcgagaagctcggcggaggcggatctggtggcggaggaagtggcggcggaggatctatccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatgggaggcggtggaagcggaggtggtggatctggcggtggcggtagcggaggcggaggttctggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccaggacatggaactggtggaaaccagacctgctggcgacggcaccttccagaaatgggcttctgtggtggtgcccctgggcaaagagcagtactacacctgtcacgtgtaccaccagggcctgcctgaacctctgacactgagatgggaaggatccatcgagttcatgtaccctccaccttacctggacaacgagagaagcaacggcaccatcatccacatcaaagagaagcacctctgtcacacccagagcagccccaagctgttttgggctcttgtggtggtggctggcgtgctgttttgttacggcctgctcgtgacagtggccctgtgtgtgatctggaccaacagcagaagaaacagaggcggccagagcgactacatgaacatgacccctagaaggcctggcctgaccagaaagccttaccagccttacgctcccgccagagatttcgccgcttatagaccaccgcggagagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 28 - protein, 654 aa, synthetic construct
  REGION 1..654: note = OVA-bait-int-AA
MARSVTLVFLVLVSLTGLYASIINFEKLGGGGSGGGGSGGGGSIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELILNMELVETRPAGDGTFQKWASVVVPLGKEQYYTCHVYHQGLPEPLTLRWEIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 29 - DNA, 1974 bp, synthetic construct
  misc_feature 1..1974: note = OVA-bait-int-NT
  source 1..1974: mol_type = other DNA
atggccagatctgtgaccctggtgttcctggtgctggtgtctctgacaggactgtacgccagcatcatcaacttcgagaagctcggcggaggcggatctggtggcggaggaagtggcggcggaggatctatccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatgggaggcggtggaagcggaggtggtggatctggcggtggcggtagcggaggcggaggttctggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccagaacatggaactggtggaaaccagacctgccggcgacggcacattccagaaatgggcatctgtggtggtgcccctgggcaaagagcagtactacacctgtcacgtgtaccaccagggcctgcctgaacctctgacactgagatgggaaggatccatcgagttcatgtaccctccaccttacctggacaacgagagaagcaacggcaccatcatccacatcaaagagaagcacctctgtcacacccagagcagccccaagctgttttgggctcttgtggtggtggctggcgtgctgttttgttacggcctgctcgtgacagtggccctgtgtgtgatctggaccaacagcagaagaaacagaggcggccagagcgactacatgaacatgacccctagaaggcctggcctgaccagaaagccttaccagccttacgctcccgccagagatttcgccgcttatagaccaccgcggagagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 30 - protein, 654 aa, synthetic construct
  REGION 1..654: note = OVA-bait-null-AA
MARSVTLVFLVLVSLTGLYASIINFEKLGGGGSGGGGSGGGGSIQKTPQIQVYSRHPPENGKPNILNCYVTQFHPPHIEIQMLENGKKIPKVEMSDMSFSKDWSFYILAHTEFTPTETDTYACRVKHASMAEPKTVYWDRDMGGGGSGGGGSGGGGSGGGGSGPHSLRYFVTAVSRPGLGEPRYMEVGYVDDTEFVRFDSDAENPRYEPRARWMEQEGPEYWERETQKAKGNEQSFRVDLRTLLGYYNQSKGGSHTIQVISGCEVGSDGRLLRGYQQYAYDGCDYIALNEDLKTWTAADMAALITKHKWEQAGEAERLRAYLEGTCVEWLRRYLKNGNATLLRTDSPKAHVTHHSRPEDKVTLRCWALGFYPADITLTWQLNGEELIQNMELVETRPAGDGTFQKWASVVVPLGKEQYYTCHVYHQGLPEPLTLRWEIEFMYPPPYLDNERSNGTIIHIKEKHLCHTQSSPKLFWALVVVAGVLFCYGLLVTVALCVIWTNSRRNRGGQSDYMNMTPRRPGLTRKPYQPYAPARDFAAYRPRAKFSRSAETAANLQDPNQLFNELNLGRREEFDVLEKKRARDPEMGGKQQRRRNPQEGVYNALQKDKMAEAYSEIGTKGERRRGKGHDGLFQGLSTATKDTFDALHMQTLAPR

SEQ ID NO: 31 - DNA, 1974 bp, synthetic construct
  misc_feature 1..1974: note = OVA-bait-null-NT
  source 1..1974: mol_type = other DNA
atggccagatctgtgaccctggtgttcctggtgctggtgtctctgacaggactgtacgccagcatcatcaacttcgagaagctcggcggaggcggatctggtggcggaggaagtggcggcggaggatctatccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatgggaggcggtggaagcggaggtggtggatctggcggtggcggtagcggaggcggaggttctggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatcctgaacatggaactggtggaaaccagacctgccggcgacggcacattccagaaatgggcatctgtggtggtgcccctgggcaaagagcagtactacacctgtcacgtgtaccaccagggcctgcctgaacctctgacactgagatgggaaggatccatcgagttcatgtaccctccaccttacctggacaacgagagaagcaacggcaccatcatccacatcaaagagaagcacctctgtcacacccagagcagccccaagctgttttgggctcttgtggtggtggctggcgtgctgttttgttacggcctgctcgtgacagtggccctgtgtgtgatctggaccaacagcagaagaaacagaggcggccagagcgactacatgaacatgacccctagaaggcctggcctgaccagaaagccttaccagccttacgctcccgccagagatttcgccgcttatagaccaccgcggagagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 32 - protein, 654 aa, synthetic construct; identical to SEQ ID NO: 20
  REGION 1..654: note = OVA-bait-CD3full-AA

SEQ ID NO: 33 - DNA, 1974 bp, synthetic construct; identical to SEQ ID NO: 21
  misc_feature 1..1974: note = OVA-bait-CD3full-NT
  source 1..1974: mol_type = other DNA

SEQ ID NO: 34 - protein, 22 aa, synthetic construct
  REGION 1..22: note = P2A-AA
GSGATNFSLLKQAGDVEENPGP

SEQ ID NO: 35 - protein, 41 aa, Homo sapiens
RSKRSRLLHSDYMNMTPRRPGPTRKHYQPYAPPRDFAAYRS

SEQ ID NO: 36 - DNA, 60 bp, Mus musculus
  source 1..60: mol_type = other DNA
atggccagatctgtgaccctggtgttcctggtgctggtgtctctgacaggactgtacgcc

SEQ ID NO: 37 - DNA, 24 bp, synthetic construct
  misc_feature 1..24: note = OVA-1-N4-NT
  source 1..24: mol_type = other DNA
agcatcatcaacttcgagaagctc

SEQ ID NO: 38 - skipped

SEQ ID NO: 39 - DNA, 24 bp, synthetic construct
  misc_feature 1..24: note = OVA-Q4-NT
  source 1..24: mol_type = other DNA
agcatcatccagttcgagaagctc

SEQ ID NO: 40 - DNA, 24 bp, synthetic construct
  misc_feature 1..24: note = OVA-T4-NT
  source 1..24: mol_type = other DNA
agcatcatcaccttcgagaagctc

SEQ ID NO: 41 - DNA, 24 bp, synthetic construct
  misc_feature 1..24: note = OVA-V4-NT
  source 1..24: mol_type = other DNA
agcatcatcgtgttcgagaagctc

SEQ ID NO: 42 - DNA, 297 bp, Mus musculus
  source 1..297: mol_type = other DNA
atccagaaaacccctcagatccaggtgtacagcagacaccctcctgagaacggcaagcccaacatcctgaactgctacgtgacccagtttcaccctcctcacatcgagatccagatgctggaaaacggcaagaagatccccaaggtcgagatgagcgacatgagcttcagcaaggactggtccttctacatcctggctcacaccgagttcacccctaccgagacagacacctacgcctgcagagtgaagcacgcctctatggccgagcctaagaccgtgtactgggacagagatatg

SEQ ID NO: 43 - DNA, 825 bp, Mus musculus
  source 1..825: mol_type = other DNA
ggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccaggacatggaactggtggaaaccagacctgctggcgacggcaccttccagaaatgggcttctgtggtggtgcccctgggcaaagagcagtactacacctgtcacgtgtaccaccagggcctgcctgaacctctgacactgagatgggaa

SEQ ID NO: 44 - DNA, 825 bp, Mus musculus
  source 1..825: mol_type = other DNA
ggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatccagaacatggaactggtggaaaccagacctgccggcgacggcacattccagaaatgggcatctgtggtggtgcccctgggcaaagagcagtactacacctgtcacgtgtaccaccagggcctgcctgaacctctgacactgagatgggaa

SEQ ID NO: 45 - DNA, 825 bp, Mus musculus
  source 1..825: mol_type = other DNA
ggacctcacagcctgagatacttcgtgaccgccgtgtctagacctggccttggcgagcccagatatatggaagtgggctacgtggacgacacagagttcgtcagattcgacagcgacgctgagaaccccagatacgagcctagagccagatggatggaacaagagggccccgagtattgggagagagagacacagaaggccaagggcaacgagcagagcttcagagtggacctgagaacactgctgggctactacaaccagagcaaaggcggcagccacaccatccaagtgatcagcggatgtgaagtgggcagcgacggcagactgctgagaggataccagcagtacgcctacgacggctgcgactatatcgccctgaacgaggacctgaaaacctggacagccgccgatatggccgctctgatcaccaagcacaagtgggagcaagctggcgaggccgagagactgagagcttacctggaaggcacctgtgtggaatggctgcggagatacctgaagaacggaaacgccacactgctgaggacagacagccctaaggctcacgtgacacaccacagcagacccgaggacaaagtgaccctgagatgctgggctctgggcttctaccctgccgacatcacactgacctggcagctgaatggcgaggaactgatcctgaacatggaactggtggaaaccagacctgccggcgacggcacattccagaaatgggcatctgtggtggtgcccctgggcaaagagcagtactacacctgtcacgtgtaccaccagggcctgcctgaacctctgacactgagatgggaa

SEQ ID NO: 46 - DNA, 81 bp, Homo sapiens
  source 1..81: mol_type = other DNA
ttctgggtgctcgttgttgttggcggcgtgctggcctgttacagcctgctggttaccgtggccttcatcatcttttgggtg

SEQ ID NO: 47 - DNA, 81 bp, Mus musculus
  source 1..81: mol_type = other DNA
ttttgggctcttgtggtggtggctggcgtgctgttttgttacggcctgctcgtgacagtggccctgtgtgtgatctggacc

SEQ ID NO: 48 - DNA, 123 bp, Homo sapiens
  source 1..123: mol_type = other DNA
cgaagcaagcggagccggggaggacacagcgactacatgaacatgacccctcggaggccaggccccaccagaaagcactaccagccctacgcccctccccgggactttgccgcctatcggagc

SEQ ID NO: 49 - DNA, 123 bp, Homo sapiens
  source 1..123: mol_type = other DNA
cgaagcaagcggagccggctgctgcacagcgactacatgaacatgacccctcggaggccaggccccaccagaaagcactaccagccctacgcccctccccgggactttgccgcctatcggagc

SEQ ID NO: 50 - DNA, 123 bp, Mus musculus
  source 1..123: mol_type = other DNA
aacagcagaagaaacagaggcggccagagcgactacatgaacatgacccctagaaggcctggcctgaccagaaagccttaccagccttacgctcccgccagagatttcgccgcttatagacca

SEQ ID NO: 51 - DNA, 342 bp, Homo sapiens
  source 1..342: mol_type = other DNA
gatatcagggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 52 - DNA, 336 bp, Homo sapiens
  source 1..336: mol_type = other DNA
agggtgaagttcagcaggagcgccgacgcccccgcttatcaacagggccagaaccagctgtacaacgagctgaacctcggcagaagagaggagtatgacgtgctggacaagaggaggggcagggaccctgagatgggcggcaagcctagaagaaagaacccccaggaaggcctctacaacgaactgcagaaggacaagatggccgaggcctacagcgagatcggcatgaaaggcgagagaaggaggggaaagggacatgacggcctgtaccagggactctccacagccaccaaggacacctacgatgccctgcacatgcaggctctgccccctaga

SEQ ID NO: 53 - DNA, 339 bp, Mus musculus
  source 1..339: mol_type = other DNA
agagccaagttctctagatctgccgagacagccgccaacctgcaggaccctaaccagctgttcaacgagctgaacctggggagaagagaagagttcgacgtgctggaaaagaagagagccagagatcccgagatgggcggcaagcagcagagaagaagaaaccctcaagagggcgtgtacaacgctctgcagaaagacaagatggccgaggcctacagcgagatcggaactaagggcgaacgcagaagaggcaagggccacgatggactgtttcagggcctgagcacagccaccaaggacacattcgatgccctgcacatgcagacactggcccctaga

SEQ ID NO: 54 - DNA, 339 bp, Mus musculus
  source 1..339: mol_type = other DNA
agagccaagttctccagatctgctgagacagccgccaacctgcaggaccctaaccagctgtacaacgagctgaacctggggagaagagaagagtacgacgtgctggaaaagaagagggccagagatcctgagatgggcggcaagcagcagagaagaagaaaccctcaagaaggcgtgtacaacgccctgcagaaagacaagatggccgaggcctacagcgagatcggaactaagggcgaacgcagaagaggcaagggccacgatggactgtaccagggcctgtctacagccaccaaggacacatacgatgccctgcacatgcagacactggcccctaga

SEQ ID NO: 55 - DNA, 66 bp, synthetic construct
  misc_feature 1..66: note = P2A-NT
  source 1..66: mol_type = other DNA
ggaagcggcgctaccaacttctccctgctcaagcaggctggcgatgtggaggagaaccccggcccc

SEQ ID NO: 56 - protein, 8 aa, synthetic construct
  REGION 1..8: note = OVA-A2-AA
SAINFEKL

SEQ ID NO: 57 - protein, 15 aa, synthetic construct
  REGION 1..15: note = G4S3-linker-AA
GGGGSGGGGSGGGGS

SEQ ID NO: 58 - protein, 20 aa, synthetic construct
  REGION 1..20: note = G4S4-linker-AA
GGGGSGGGGSGGGGSGGGGS

SEQ ID NO: 59 - skipped

SEQ ID NO: 60 - DNA, 44 bp, synthetic construct
  misc_feature 1..44: note = G4S3-linker-NT
  source 1..44: mol_type = other DNA
gcggaggcggatctggtggcggaggaagtggcggcggaggatct

SEQ ID NO: 61 - DNA, 59 bp, synthetic construct
  misc_feature 1..59: note = G4S4-linker-NT
  source 1..59: mol_type = other DNA
gaggcggtggaagcggaggtggtggatctggcggtggcggtagcggaggcggaggttct

SEQ ID NO: 62 - protein, 8 aa, synthetic construct
  VARIANT 2: note = X is A or I
  VARIANT 3: note = X is I or Y
  VARIANT 4: note = X is N, Q, T or V
SXXXFEKL

SEQ ID NO: 63 - protein, 36 aa, Mus musculus
IEFMYPPPYLDNERSNGTIIHIKEKHLCHTQSSPKL

SEQ ID NO: 64 - DNA, 108 bp, Mus musculus
  source 1..108: mol_type = other DNA
atcgagttcatgtaccctccaccttacctggacaacgagagaagcaacggcaccatcatccacatcaaagagaagcacctctgtcacacccagagcagccccaagctg

SEQ ID NO: 65 - protein, 20 aa, synthetic construct
MSRSVALAVLALLSLSGLEA

SEQ ID NO: 66 - protein, 9 aa, synthetic construct
SLLMWITQC

SEQ ID NO: 67 - protein, 99 aa, synthetic construct [sequence omitted]

SEQ ID NO: 68 - protein, 283 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIP

SEQ ID NO: 69 - protein, 283 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWVAVVVPSGQEQRYTCHVQHEGLPKPLTLRWEPSSQPTIP

SEQ ID NO: 70 - DNA, 15 bp, synthetic construct
  source 1..15: mol_type = other DNA
gcgasggggsggggs

SEQ ID NO: 71 - protein, 43 aa, synthetic construct
GAAAIEVMYPPPYLDNEKSNGTIIHVKGKHLCPSPLFPGPSKP

SEQ ID NO: 72 - protein, 113 aa, synthetic construct
VKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 73 - DNA, 60 bp, synthetic construct
  source 1..60: mol_type = other DNA
atgtcacggagcgttgccttggctgtcttggcgcttttgtctctgtccggactagaggct

SEQ ID NO: 74 - DNA, 27 bp, synthetic construct
  source 1..27: mol_type = other DNA
agtctgctcatgtggatcacacaatgt

SEQ ID NO: 75 - DNA, 297 bp, synthetic construct
  source 1..297: mol_type = other DNA
atccaacgcacaccaaagattcaagtctatagccgacatcccgccgaaaatggcaagagtaactttctgaactgttacgtgtccggatttcatccgtctgatattgaagttgacctgcttaagaatggcgaacggattgagaaagtggaacattctgacctctctttctctaaagactggtccttttatctattgtactacaccgagtttacaccaacggaaaaggacgaatacgcttgtcgagtcaaccacgtcacactgtcccaaccgaaaattgtgaagtgggatcgggatatg

SEQ ID NO: 76 - DNA, 849 bp, synthetic construct
  source 1..849: mol_type = other DNA
gggagccactcaatgaggtacttctttactagcgtaagtaggccgggtagaggtgaaccaagatttattgcagtcgggtacgttgacgacacacagttcgtgagattcgatagtgacgccgcgtcccaacggatggaaccacgagcaccctggatagaacaagaaggcccagaatactgggacggtgaaacaaggaaagtaaaagcacatagccagacccaccgcgttgatctcggtacattgcggggttgctataatcaatccgaagccgggtctcacacggtgcaaaggatgtacggatgtgatgttggcagtgactggagatttctcagagggtatcaccaatacgcctatgatggcaaagactatattgcgcttaaggaagatctccgttcctggactgctgcagatatggcagctcaaacaacgaaacataaatgggaagcagcgcatgtagccgagcagttgcgagcttacctggaaggcacatgtgtggaatggctcaggcgatacctggaaaatggaaaagagacactccagcgaactgatgctcccaaaactcatatgactcatcatgcggtatccgatcatgaagctactctgcgctgctgggcactgagcttttatccagccgagatcactctgacctggcaacgagatggggaagaccaaactcaggacaccgaactcgtggaaacaagacccgcaggcgatggaaccttccaaaagtgggcggcagtggtcgttccaagcgggcaagagcaacgatacacatgtcacgttcaacacgaaggactccctaaaccgctgacacttcgatgggagccctcaagtcagcctacaataccg

SEQ ID NO: 77 - DNA, 849 bp, synthetic construct
  source 1..849: mol_type = other DNA
gggagccactcaatgaggtacttctttactagcgtaagtaggccgggtagaggtgaaccaagatttattgcagtcgggtacgttgacgacacacagttcgtgagattcgatagtgacgccgcgtcccaacggatggaaccacgagcaccctggatagaacaagaaggcccagaatactgggacggtgaaacaaggaaagtaaaagcacatagccagacccaccgcgttgatctcggtacattgcggggttgctataatcaatccgaagccgggtctcacacggtgcaaaggatgtacggatgtgatgttggcagtgactggagatttctcagagggtatcaccaatacgcctatgatggcaaagactatattgcgcttaaggaagatctccgttcctggactgctgcagatatggcagctcaaacaacgaaacataaatgggaagcagcgcatgtagccgagcagttgcgagcttacctggaaggcacatgtgtggaatggctcaggcgatacctggaaaatggaaaagagacactccagcgaactgatgctcccaaaactcatatgactcatcatgcggtatccgatcatgaagctactctgcgctgctgggcactgagcttttatccagccgagatcactctgacctggcaacgagatggggaagaccaaactcaggacaccgaactcgtggaaacaagacccgcaggcgatggaaccttccaaaagtgggtggcagtggtcgttccaagcgggcaagagcaacgatacacatgtcacgttcaacacgaaggactccctaaaccgctgacacttcgatgggagccctcaagtcagcctacaataccg

SEQ ID NO: 78 - DNA, 60 bp, synthetic construct
  source 1..60: mol_type = other DNA
ggtggtggcggaagcggtggtggtggatcaggtggcggcggcagtggtggtggtggatct

SEQ ID NO: 79 - DNA, 45 bp, synthetic construct
  source 1..45: mol_type = other DNA
ggatgtggtgcttctggcggtggcggaagtggtggtggcgggagc

SEQ ID NO: 80 - DNA, 129 bp, synthetic construct
  source 1..129: mol_type = other DNA
ggcgccgctgctattgaagtgatgtaccctcctccttacctggacaacgagaagtccaacggcaccatcatccacgtgaagggcaagcacctgtgtccttctccactgttccccggacctagcaagcct

SEQ ID NO: 81 - DNA, 81 bp, synthetic construct
  source 1..81: mol_type = other DNA
ttctgggtgctcgttgttgttggcggcgtgctggcctgttattccctgctggttaccgtggccttcatcatcttttgggtc

SEQ ID NO: 82 - DNA, 120 bp, synthetic construct
  source 1..120: mol_type = other DNA
cgaagcaagcggagccggctgctgcacagcgactacatgaacatgacccctagacggcccggaccaaccagaaagcactaccagccttacgctcctcctagagacttcgccgcctaccgg

SEQ ID NO: 83 - DNA, 339 bp, synthetic construct
  source 1..339: mol_type = other DNA
gtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatcc

SEQ ID NO: 84 - protein, 673 aa, synthetic construct
MSRSVALAVLALLSLSGLEASLLMWITQCGCGASGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGCYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPTSGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGS

SEQ ID NO: 85 - DNA, 2022 bp, synthetic construct
  source 1..2022: mol_type = other DNA
atgtcacggagcgttgccttggctgtcttggcgcttttgtctctgtccggactagaggctagtctgctcatgtggatcacacaatgtggatgtggtgcttctggcggtggcggaagtggtggtggcgggagcatccaacgcacaccaaagattcaagtctatagccgacatcccgccgaaaatggcaagagtaactttctgaactgttacgtgtccggatttcatccgtctgatattgaagttgacctgcttaagaatggcgaacggattgagaaagtggaacattctgacctctctttctctaaagactggtccttttatctattgtactacaccgagtttacaccaacggaaaaggacgaatacgcttgtcgagtcaaccacgtcacactgtcccaaccgaaaattgtgaagtgggatcgggatatgggtggtggcggaagcggtggtggtggatcaggtggcggcggcagtggtggtggtggatctgggagccactcaatgaggtacttctttactagcgtaagtaggccgggtagaggtgaaccaagatttattgcagtcgggtacgttgacgacacacagttcgtgagattcgatagtgacgccgcgtcccaacggatggaaccacgagcaccctggatagaacaagaaggcccagaatactgggacggtgaaacaaggaaagtaaaagcacatagccagacccaccgcgttgatctcggtacattgcggggttgctataatcaatccgaagccgggtctcacacggtgcaaaggatgtacggatgtgatgttggcagtgactggagatttctcagagggtatcaccaatacgcctatgatggcaaagactatattgcgcttaaggaagatctccgttcctggactgctgcagatatggcagctcaaacaacgaaacataaatgggaagcagcgcatgtagccgagcagttgcgagcttacctggaaggcacatgtgtggaatggctcaggcgatacctggaaaatggaaaagagacactccagcgaactgatgctcccaaaactcatatgactcatcatgcggtatccgatcatgaagctactctgcgctgctgggcactgagcttttatccagccgagatcactctgacctggcaacgagatggggaagaccaaactcaggacaccgaactcgtggaaacaagacccgcaggcgatggaaccttccaaaagtgggcggcagtggtcgttccaagcgggcaagagcaacgatacacatgtcacgttcaacacgaaggactccctaaaccgctgacacttcgatgggagccctcaagtcagcctacaataccgactagtggcgccgctgctattgaagtgatgtaccctcctccttacctggacaacgagaagtccaacggcaccatcatccacgtgaagggcaagcacctgtgtccttctccactgttccccggacctagcaagcctttctgggtgctcgttgttgttggcggcgtgctggcctgttattccctgctggttaccgtggccttcatcatcttttgggtccgaagcaagcggagccggctgctgcacagcgactacatgaacatgacccctagacggcccggaccaaccagaaagcactaccagccttacgctcctcctagagacttcgccgcctaccggtctagagtgaagttcagcagatccgccgacgctcctgcctatcagcagggacagaaccagctgtacaacgagctgaacctggggagaagagaagagtacgacgtgctggacaagcggagaggcagagatcctgagatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagaggatcctaa

SEQ ID NO: 86 - protein, 5 aa, synthetic construct
GCGAS

(19) United States
(12) Patent Application Publication
(10) Pub. No.: US 2025/0304913 A1
(43) Pub. Date: Oct. 2, 2025

(54) CHIMERIC ANTIGEN RECEPTOR T CELLS AND METHODS OF USE THEREOF

(71) Applicant: The Regents of the University of Colorado, A Body Corporate, Denver, CO (US)

(72) Inventors: Mathew Prashanth FRANCIS, Denver, CO (US); Terry J. FRY, Bethesda, MD (US); Michael Craig YARNELL, Denver, CO (US)

(21) Appl. No.: 18/853,458

(22) PCT Filed: Apr. 6, 2023
(86) PCT No.: PCT/US2023/065479

§ 371 Date: Oct. 2, 2024

Related U.S. Application Data
(60) Provisional application No. 63/327,890, filed on Apr. 6, 2022.
(60) Provisional application No. 63/339,100, filed on May 6, 2022.

(51) Int. Cl. C12N 5/0783; A61K 35/12; A61K 35/17; A61K 40/11; A61K 40/31; A61K 40/41; A61P 37/02; C12N 5/0789

(52) U.S. Cl. CPC C12N 5/0636; A61K 35/17; A61K 40/11; A61K 40/31; A61K 40/416; A61P 37/02; C12N 5/0647; A61K 2035/124; A61K 2239/21; A61K 2239/22

(57) ABSTRACT

The disclosure describes T cells that express chimeric antigen receptors (CARs), as well as pharmaceutical compositions comprising T cells and methods of making and using such T cells. Particularly, this disclosure describes T cells expressing a CAR that specifically bind to pathologic T-cells, and methods of use in the treatment of autoimmune disease, transplant rejection, T cell malignancies, and chronic inflammatory disease.

### RELATED APPLICATIONS

This application is a U.S. National Phase application, filed under 35 U.S.C. § 371, of International Application No. PCT/US2023/065479, filed Apr. 6, 2023, which claims priority to, and the benefit of, U.S. Provisional Patent Application No. 63/327,890, filed on Apr. 6, 2022, and U.S. Provisional Patent Application No. 63/339,100, filed on May 6, 2022, each of which is incorporated herein by reference in its entirety.

### GOVERNMENT SUPPORT

This invention was made with government support under Grant No. 5T32DK007038-45 awarded by the National Institutes of Health (T32 Grant-Institutional National Research Service Award). The government has certain rights in the invention.

### FIELD OF INVENTION

The present invention relates generally to the fields of molecular biology, immunology, oncology and medicine. More particularly, it concerns immune cells expressing chimeric antigen receptors, such as chimeric antigen receptors that bind to a target protein.

### REFERENCE TO AN ELECTRONIC SEQUENCE LISTING

This application contains a Sequence Listing that has been submitted electronically as an XML file named 58666-0007US1_SL_ST26.xml. The XML file, created on Apr. 30, 2025, is 117,428 bytes in size. The material in the XML file is hereby incorporated by reference in its entirety.

### BACKGROUND OF THE INVENTION

Aberrant or dysregulated immune responses represent the underlying mechanisms of numerous pathological conditions. Such conditions include cancers, autoimmune disorders, acute and chronic rejection of transplanted organs, graft versus host disease, allergic diseases, and conditions characterized by chronic inflammation.

Autoimmunity is a condition where the immune system mistakenly recognizes host tissue or cells as foreign. Autoimmune diseases affect millions of individuals worldwide. Common autoimmune disorders include type 1 diabetes mellitus, systemic lupus erythematosus, psoriasis and psoriatic arthritis, rheumatoid arthritis, (Hashimoto's) autoimmune thyroiditis, inflammatory bowel diseases such as ulcerative colitis and Crohn's disease, autoimmune hepatitis, primary biliary cholangitis, pernicious anemia, Celiac disease, autoimmune vasculitis, Sjogren's disease, and multiple sclerosis. Aberrant or pathological immune activation underlies diseases, such as autoimmune diseases, transplantation graft rejection, allergy, and asthma. These immune activation disorders are prevalent and contribute to significant morbidity and mortality.

Specifically, pathologic T cell reactivity is a critical component of many diseases, including autoimmune diseases, such as type 1 diabetes mellitus and rheumatoid arthritis, T cell leukemia, graft vs host disease, and transplant rejection. Recent discoveries have also shown their role in diseases such as hypertension and cardiovascular disease. Currently approved treatments have limited abilities in distinguishing between targeting of pathogenic T-cells vs non-pathogenic T cells. While such pan-T cell treatments can be effective, they have significant risks such as infection, malignancy, metabolic and cardiovascular disease, with many patients dying from complications of treatment instead of the original disease itself.

Few therapies exist that are sufficiently potent while maintaining specificity. Accordingly, few therapies exist to treat such pathologic T cell diseases of the immune system, and those that do tend to have substantial side effects and rarely target the underlying mechanism of disease. There is a need for effective targeted treatment of immune activation disorders with minimal or no side effects. The present invention addresses these unmet needs in the art.

### SUMMARY OF INVENTION

The present disclosure provides a chimeric antigen receptor (CAR) comprising: (a) an ectodomain comprising i. a beta-2 microglobulin leader peptide (B2ML), ii. a cognate peptide that is recognized by a CD8⁺ T-cell Receptor (P), iii. at least one linker domain (L), iv. a beta-2 microglobulin peptide (B2M), v. a MHC class I (MHCI), a HLA-A, a HLA-B or a HLA-C; and vi. a stalk/hinge domain (b) a transmembrane domain: (c) at least one costimulatory domain; and (d) an intracellular signaling domain.

The present disclosure provides a cell comprising a chimeric antigen receptor (CAR) comprising: (a) an ectodomain comprising: i. a beta-2 microglobulin leader peptide (B2ML), ii. a cognate peptide that is recognized by a CD8+ T-cell Receptor (P), iii. at least one linker domain, iv. a beta-2 microglobulin peptide (B2M), and v. a MHCI, a HLA-A, a HLA-B or a HLA-C; and vi. a stalk/hinge domain (b) a transmembrane domain: (c) at least one costimulatory domain; and (d) a intracellular signaling domain.

In some embodiments, the CAR comprises: (a) an ectodomain comprising: i. a human beta-2 microglobulin leader peptide (B2ML), ii. a cognate peptide that is recognized by a human CD8+ T-cell Receptor (P), iii. at least one linker domain, iv. a human beta-2 microglobulin peptide (B2M), and v. a HLA-A, a HLA-B or a HLA-C; and vi. a stalk/hinge domain (b) a human transmembrane domain: (c) at least one human costimulatory domain; and (d) a human intracellular signaling domain.

In some embodiments, the ectodomain comprises the following in the N-terminal to C-terminal direction: N-term-B2ML-P-(L)ₓ-B2M-(L)_{y}-(MHCI/HLA-A/HLA-B/HLA-C)-stalk/hinge-C-term wherein x is any integer between 0-5; and wherein y is any integer between 0)-5.

In some embodiments, the ectodomain comprises the following in the N-terminal to C-terminal direction: N-term-B2ML-P-(Linker 1)ₓ-(Linker 2)_{y}-B2M-(Linker 2) 2-(MHC-I/HLA-A/HLA-B/HLA-C)-stalk/hinge-C-term, wherein x is any integer between 0-5: wherein y is any integer between 0-5; and wherein z is any integer between 0-5.

In some embodiments, the cognate peptide is isolated or derived from ovalbumin. neoantigen or autoantigen of an autoimmune disease, neoantigen or alloantigen of transplant rejection, or cognate antigen of other pathogenic T cells. In some embodiments, the cognate peptide comprises the amino acid sequence SX₁X₂X₃FEKL (SEQ ID NO: 62), wherein X₁ is A or I: X₂ is I or Y; and X₃ is N. Q. T or V. In some embodiments, the cognate peptide comprises the amino acid sequence of SEQ ID NO: 7-10, or 56.

In some embodiments, the B2ML is a mouse B2ML or a human B2ML. In some embodiments, the B2ML comprises the amino acid sequence of SEQ ID NO: 1. In some embodiments, the human B2ML comprises the amino acid sequence of SEQ ID NO: 73.

In some embodiments, the at least one linker domain comprises the amino acid sequence of SEQ ID NO: 11, 57, 58, 70 or 86.

In some embodiments, the B2M is a mouse B2M or a human B2M. In some embodiments, the mouse B2M comprises the amino acid sequence of SEQ ID NO: 2. In some embodiments, the human B2M comprises the amino acid sequence of SEQ ID NO: 75.

In some embodiments, the ectodomain of (a) comprises a MHCI. In some embodiments, the MHCI comprises the amino acid sequence of SEQ ID NO: 3. In some embodiments, the MHCI comprises a mutation in the epitope binding domain of the MHCI. In some embodiments, the MHCI comprises a mutation in the CD8 binding site domain of the MHCI. In some embodiments, the MHCI comprises the amino acid sequence of SEQ ID NO: 3-5.

In some embodiments, the ectodomain of (a) comprises a HLA-A. HLA-B or HLA-C. In some embodiments, the ectodomain of (a) comprises a HLA-A. In some embodiments, the HLA-A comprises the amino acid sequence of SEQ ID NO: 76. In some embodiments, the MHCI comprises a mutation in the CD8 binding domain of the HLA-A. HLA-B or HLA-C. In some embodiments, the HLA-A comprises at least one mutation in the CD8 binding domain. In some embodiments, the HLA-A comprising at least one mutation in the CD8 binding domain comprises the amino acid sequence of SEQ ID NO: 77.

In some embodiments, the ectodomain of (a) comprises a HLA-A. a HLA-B or a HLA-C.

In some embodiments, the stalk/hinge domain of (a) comprises a CD28. CD8. CD8a. or CD8 beta extracellular domain. In some embodiments, the stalk/hinge domain comprises a CD28 stalk/hinge domain. In some embodiments, the CD28 stalk/hinge domain comprises the amino acid sequence of SEQ ID NO: 63 or 71.

In some embodiments, the transmembrane domain comprises a CD28, CD8, CD8α, CD8 beta, CD3-epsilon, CD3-delta, CD3-gamma, CD3%, CD4, 4-1BB, OX40, ICOS, PD-1, LAG-3, 2B4 or BTLA transmembrane domain or a portion thereof. In some embodiments, the transmembrane domain comprises a CD28 transmembrane domain. In some embodiments, the CD28 transmembrane domain comprises the amino acid sequence of SEQ ID NO: 12, 13 or 81.

In some embodiments, the at least one costimulatory domain comprises a CD28, 4-IBB (CD137), CD97, CD11a-CD18, CD2, ICOS, CD27, CD154, CD8α, OX40 (CD134) co-stimulatory domain or a portion thereof. In some embodiments, the at least one costimulatory domain comprises a CD28 costimulatory domain. In some embodiments, the CD28 costimulatory domain comprises the amino acid sequence of SEQ ID NO: 14 or 15.

In some embodiments, the intracellular signaling domain comprises a CD33 intracellular signaling domain. In some embodiments, the CD3ξ intracellular signaling domain comprises a mutation in at least one of the ITAM domains of the CD3ξ intracellular signaling domain. In some embodiments, the CD35 intracellular signaling domain comprises the amino acid sequence of SEQ ID NO: 16-19 or 72.

In some embodiments, the CAR comprises: (a) an ectodomain comprising i. a beta-2 microglobulin leader peptide (B2ML) comprising the amino acid sequence of SEQ ID NO: 1. ii. a cognate peptide that is recognized by a CD8 T-cell Receptor (P) comprising the amino acid sequence of SEQ ID NO: 6, 7, 8, 9, 10, 56 or 62, iii. at least one linker domain (L) comprising the amino acid sequence of SEQ ID NO: 11, 57 or 58, iv. a beta-2 microglobulin peptide (B2M) comprising the amino acid sequence of SEQ ID NO: 2, and v. a MHC class I (MHCI) comprising the amino acid sequence of SEQ ID NOs: 3-5; and vi. a stalk/hinge domain comprising amino acid SEQ ID NO: 63: (b) a transmembrane domain comprising the amino acid sequence of SEQ ID NO: 12: (c) a costimulatory domain comprising the amino acid sequence of SEQ ID NO: 14; and (d) a intracellular signaling domain comprising the amino acid sequence of SEQ ID NO: 18 or 19.

In some embodiments, the CAR comprises the amino acid sequence of SEQ ID NOs: 20, 22, 24, 26, 28, 30, 32 or 84.

The present disclosure provides a polynucleotide comprising a nucleic acid encoding any one of the CARs described herein. The present disclosure also provides a vector comprising the polynucleotide.

In some embodiments, the cell is a T-cell, a hematopoietic progenitor cell, a peripheral blood (PB) derived T-cell or an umbilical cord blood (UCB) derived T-cell. In some embodiments, the cell is a CD8+ T-cell. The present disclosure also provides composition comprising the cell described herein and a pharmaceutically acceptable carrier. The present disclosure provides a method of targeting a specific group of CD8+ T-cell comprising providing a population of the immune cells described herein to a subject in need thereof.

The disclosure provides a pharmaceutical composition comprising: i) a population of cells comprising about 1.0×10⁵ to about 1.0×10⁹ of the cells of any one of claims 1-13; and ii) a pharmaceutically acceptable carrier, wherein the pharmaceutical composition is suitable for administration to a human subject.

The disclosure provides a method of inducing cell death of a population of CD8+ pathologic T-cells in a human subject in need thereof, the method comprising: administering to the human subject a therapeutically effective amount of the pharmaceutical composition of the disclosure under a condition suitable for binding of the plurality of cells of the pharmaceutical composition with the plurality of CD8+ pathologic T cells of the human subject, thereby inducing the cell death of the population of CD8+ pathologic T-cells in the human subject.

In some embodiments, the cell death of the population of CD8+ pathologic T cells in the human subject is about 2-fold to about 100-fold higher than the cell death of a population of CD8+ pathologic T cells in a human subject that has not been administered with the pharmaceutical composition of the disclosure. In some embodiments, the condition is an autoimmune disease, transplant rejection, allergic disease, malignancy, or a chronic inflammatory disease.

### BRIEF DESCRIPTION OF THE DRAWINGS

FIG. 1A is a schematic depicting an exemplary embodiment of the MHC I bait chimeric antigen receptor (CAR) described herein.

FIG. 1B is a schematic depicting the polypeptide domains of an exemplary embodiment of the MHC I bait CAR described herein.

FIG. 1C is a schematic depicting an exemplary αVβ5 scFv CAR.

FIG. 1D is a schematic depicting exemplary variants of the bait CAR described herein. Mutations can be made in the cognate peptide (epitope binding mutations), the CD8 costimulatory binding site of the MHCI, or the CD3z intracellular domain (zeta variants).

FIG. 1E is a schematic depicting the CD3z intracellular domain variants (zeta variants). The “Lx3” variant has 3 live ITAM domains. The “Zi2” variant has 2 live ITAM domains. The “Dx3” variant has 0 live ITAM domains.

FIGS. 2A-2C show a series of graphs depicting expression of the exemplary MHC I bait CAR on the surface of T cells transduced with a nucleotide sequence encoding the exemplary bait chimeric antigen receptor. Expression was quantified using flow cytometry. FIG. 2A shows expression of exemplary CAR reporter protein. FIG. 2B shows expression of the exemplary CAR linker domain. FIG. 2C shows expression of the MHC I and cognate peptide.

FIGS. 3A-3B show a series of flow cytometry contour plots depicting the recognition and activation (degranulation) of target (OT-I) T cells in vitro by exemplary MHC I bait CAR T cells. CD107a expression was used as a marker of T cell activation and quantified using flow cytometry. FIG. 3A depicts activation of OT-I cells incubated alone. FIG. 3B depicts activation of OT-I cells co-cultured with exemplary bait CAR T cells.

FIGS. 4A-4B show a series of flow cytometry contour plots depicting the activation (degranulation) of exemplary bait CAR T cells in vitro. CD107a expression was used as a marker of T cell activation and quantified using flow cytometry. FIG. 4A shows activation of exemplary bait CAR T cells incubated alone. FIG. 4B shows exemplary bait CAR T cells incubated in coculture with target OT-I T cells.

FIGS. 5A-5D show a series of graphs depicting CAR T cell activation in the presence (blue) and absence (red) of target OT-I T cells in vitro. CD69 expression was used as a marker of activation and quantified by flow cytometry. FIG. 5A shows CD69 expression in mock CAR T cells in the presence and absence of OT-I T cells. FIG. 5B shows CD69 expression in exemplary bait CAR T cells in the presence and absence of OT-I T cells. FIG. 5C shows CD25 expression in mock CAR T cells in the presence and absence of OT-IT cells. FIG. 5D shows CD25 expression in exemplary bait CAR T cells in the presence and absence of OT-I T cells.

FIGS. 6A-6D show a series of graphs depicting cytokine production in mock and exemplary bait CAR T cells. FIG. 6A shows IFN-gamma production in mock CAR T cells in the presence of OT-I T cells. FIG. 6B shows IFN-gamma production in exemplary bait CAR T cells in the presence of OT-I T cells. FIG. 6C shows IL-2 production in mock CAR T cells in the presence of T cells. FIG. 6D shows IL-2 production in exemplary bait CAR T cells in the presence of OT-I T cells.

FIGS. 7A-7B show a series of flow cytometry dot plots depicting survival of exemplary bait CAR T cells in the presence of OT-I target T cells (45.2+). Cell populations were quantified and sorted using flow cytometry. FIG. 7A shows survival of mock CAR T cells (45.1+). FIG. 7B shows survival of exemplary bait CAR T cells (45.1+).

FIGS. 8A-8I show a series of flow cytometry dot plots depicting survival of exemplary bait CAR T cells (45.1+) comprising exemplary CD3 ITAM mutations incubated in the presence of OT-I target T cells (45.2+) for 18 hours to 60 hours. Exemplary bait and target T cell populations were quantified and sorted using flow cytometry. FIG. 8A shows survival of mock CAR T cells at 18 hours. FIG. 8B shows survival of mock CAR T cells at 36 hours. FIG. 8C shows survival of mock CAR T cells at 60 hours. FIG. 8D shows survival of a first exemplary bait CAR T cell comprising a first CD3 ITAM mutation at 18 hours.

FIG. 8E shows survival of a first exemplary bait CAR T cell comprising a first CD3 ITAM mutation at 36 hours. FIG. 8F shows survival of a first exemplary bait CAR T cell comprising a first CD3 ITAM mutation at 60 hours. FIG. 8G shows survival of a second exemplary bait CAR T cell comprising a second ITAM mutation at 18 hours. FIG. 8H shows survival a second exemplary bait CAR T cell comprising a second ITAM mutation at 36 hours. FIG. 8I shows survival a second exemplary bait CAR T cell comprising a second ITAM mutation at 60 hours.

FIG. 9A shows a chart depicting OT-I target T cell sensitivity to exemplary bait CAR T cells loaded with exemplary cognate peptide mutation variants. Sensitivity is presented as EC50. Data shown from Bevans, et al: Nature. 2009, 458 (7235): 211-214.

FIGS. 10A-10E show a series of flow cytometry dot plots depicting survival of exemplary bait CAR T cells (45.1+) loaded with exemplary cognate peptide mutants, identified in the legend in FIG. 9A, and incubated in the presence of target OT-I T cells. Exemplary bait and target T cell populations were sorted and quantified using flow cytometry. FIG. 10A shows mock CAR T cells. FIG. 10B shows an exemplary bait CAR T cell (“SIIVFEKL-Zi2”) comprising a first CD3 ITAM mutation (Zi2) and a first cognate peptide mutant (SIIVFEKL). FIG. 10C shows an exemplary bait CAR T cell (“SIITFEKL-Zi2”) comprising a first CD3 ITAM mutation (Zi2) and a second cognate peptide mutant (SIITFEKL). FIG. 10D shows an exemplary bait CAR T cell (“SIIQFEKL-Zi2”) comprising a first CD3 ITAM mutation (Zi2) and a third cognate peptide mutant (SIIQFEKL). FIG. 10E shows an exemplary bait CAR T cell (“SIINFEKL-Zi2”) comprising a first CD3 ITAM mutation (Zi2) and a fourth cognate peptide mutant (SIINFEKL).

FIGS. 11A-11D show a series of graphs depicting activation of exemplary bait CAR T cells comprising exemplary CD3 ITAM mutations and exemplary CD8 binding site domain mutations and incubated in coculture with target OT-I T cells, the cell types identified in the legend depicted in FIG. 11E. FIG. 11A shows CD69 expression in exemplary bait CAR T cells compared to mock control CAR T cells. FIG. 11B shows CD69 expression in OT-I T cells in compared to mock control CAR T cells. FIG. 11C shows CD25 expression in exemplary bait CAR T cells compared to mock control CAR T cells. FIG. 11D shows CD25 expression in OT-I T cells compared to mock control CAR T cells.

FIGS. 12A-12G show a series of flow cytometry dot plots depicting survival of exemplary bait CAR T cells comprising exemplary CD8 binding site mutations and exemplary CD3 ITAM mutations and incubated in the presence of target OT-I T cells (45.2+). Cell populations are sorted and quantified using flow cytometry. FIG. 12A shows survival of mock CAR T cells (45.1+). FIG. 12B shows survival of a first exemplary bait CAR T cell comprising a first CD8 binding site mutation and a first cognate peptide mutant (45.1+). FIG. 12C shows survival of a second exemplary bait CAR T cell comprising a second CD8 binding site mutation and a second cognate peptide mutant (45.1+). FIG. 12D is a series of flow cytometry plots showing that bait CAR T cells with CD8 null binder (SCT3) decreases the activation in OT-I cells. FIG. 12E is a graph showing the quantification of the results of FIG. 12D, showing the percent of OT-I cells activated by bait CAR T cells. FIG. 12F is a series of flow cytometry plots showing that bait CAR T cells with CD8 null binder (SCT3) exhibits increased targeted killing of OT-I cells than wildtype MHCI domain (SCT1). FIG. 12G is a graph showing the quantification of the results of FIG. 12F, showing the percent of OT-I cells killed by the bait CAR T cells.

FIG. 13 shows a schematic of a first model of in vivo effector T cell depletion by exemplary bait CAR T cells.

FIGS. 14A-14D show a series of flow cytometry contour plots depicting effector T cell depletion in the spleen and liver of mice treated with exemplary bait CAR T cells and mock CAR T cells using the first in vivo model depicted in FIG. 13. FIG. 14A shows effector T cell depletion in mock CAR T cell-treated mice. FIG. 14B shows effector T cell depletion in exemplary bait CAR T cell-treated mice. FIG. 14C shows the percentage of target effector CD8 T cells in mock-treated and exemplary bait CAR T cell-treated mice.

FIG. 14D shows the percentage of IFN-gamma positive CD8 T cells in mock-treated and exemplary bait CAR-treated mice.

FIG. 15 shows a schematic of a second model of in vivo effector T cell depletion by exemplary bait CAR T cells.

FIGS. 16A-16D show a series of graphs depicting effector T cell populations in the spleen of mice treated with mock CAR T cell and exemplary bait CAR T cell in the second in vivo model depicted in FIG. 15. FIG. 16A shows the percentage of peptide/tetramer positive CD8 T cells. FIG. 16B shows the total number of peptide/tetramer-positive T cells. FIG. 16C shows the percentage of CD8 T cells from these mice which produce IFN-gamma after stimulation with peptide-tetramer complexes. FIG. 16D shows the percentage of CD8 T cells from these mice which produce IFN-gamma and IL-2 after stimulation with peptide-tetramer complexes.

FIGS. 17A-17C show a series of graphs depicting results of CAR T cell and OT-IT cell in vitro co-culture experiments. FIG. 17A shows activation displayed by CD69 staining.

FIG. 17B shows degranulation (CD107a), and FIG. 17C shows production of cytokines IFN-y and IL-2.

FIGS. 18A-18C are a series of graphs depicting results of in Vitro Analysis of Kinetics of Target Killing and Activation, as well as Cytokine Production. FIG. 18A shows the percentage of remaining OT-I T cells at different timepoints after coculture with CAR T cells over 48 hrs with cytokines. FIG. 18B shows activation of SIINFEKL-H2-K^{b} Bait CAR and FIG. 18C shows cytokine production.

FIGS. 19A-19C show results of testing CAR T cells using in vivo models with polyclonal OVA-reactive T Cells. FIG. 19A shows target cell depletion in mice given OVA-expressing Listeria monocytogenes, followed by CAR T cells 2 days later. FIG. 19B shows depletion of OVA-reactive cells, including SIINFEKL-tetramer-induced IFNγ and IFNγ/IL-2 positive cells, in mice receiving OVA vaccination plus adjuvant. FIG. 19C shows infections in OVA-vaccinated mice challenged 28 days later with LM-OVA infection.

FIG. 20A is a series of flow cytometry plots showing that maximizing the number of live ITAM domains in the CD3 zeta intracellular domain of the bait CAR T cells increases in vitro killing of OT 1 cells. Mock cells, SCT-1-Dx3 CAR T cells, SCTI-Zi2 CAR T cells, and SCT1-Lx3 CAR T cells were tested.

FIG. 20B is a graph showing the quantification of the results shown in FIG. 20A. The y axis shows the percent of surviving OT-I cells following contact with Mock cells, SCT-1-Dx3 CAR T cells, SCTI-Zi2 CAR T cells, and SCT1-Lx3 CAR T cells.

FIG. 21A is a schematic diagram of an experimental protocol to test if adoptive transfer of T cells from OVA-vaccinated mice induces Type 1 diabetes in RIP-mOVA mice.

FIG. 21B is a graph showing the blood glucose (mg/dL) levels over time of the mice tested according to FIG. 21A.

FIG. 22A is a schematic diagram of an experimental protocol to test if bait CAR (OVA-MHC-I) prevents induction of Type 1 diabetes by OVA-reactive T cells.

FIG. 22B is a graph showing the blood glucose (mg/dL) levels over time of the mice tested according to FIG. 22A.

FIG. 23A is a schematic diagram of an experimental protocol to test if bait CAR (OVA-MHC-I) prevents induction of Type 1 diabetes by using a predetermined CD4: CD8 ratio of OVA-reactive T cells.

FIG. 23B is a graph showing the blood glucose (mg/dL) levels over time of the mice tested according to FIG. 23A.

FIG. 24 is a schematic diagram showing an exemplary human bait CAR (“HLA-A2-NYESO Bait CAR”) described herein.

FIGS. 25A-25B are a series of flow cytometry plots showing the transduction efficiency of the HLA-A2-NYESO Bait CARs in Donor 22 (FIG. 25A) or Donor 23 (FIG. 25B).

FIGS. 26A-26B are a series of flow cytometry plots showing the degranulation (CD107a levels) of HLA-A2-NYESO bait CAR T cells (upper) and target T cells (lower) whether alone (left) or in co-culture with one another (right), in Donor 22 (FIG. 26A) or Donor 23 (FIG. 26B).

FIGS. 27A-27B are a series of flow cytometry plots showing the activation (CD69 levels) of HLA-A2-NYESO bait CAR T cells (upper) and target T cells (lower) whether alone (left) or in co-culture with one another (right), in Donor 22 (FIG. 27A) or Donor 23 (FIG. 27B).

FIG. 28A is a series of flow cytometry plots showing the number of target m1G4 T cells when co-cultured in vitro with mock CAR T cells or HLA-A2 NYESO bait CAR T cells.

FIG. 28B is a graph showing the percent of surviving 1G4 T cells over time upon co culturing in vitro with mock CAR T cells or HLA-A2 NYESO bait CAR T cells.

FIG. 29A is a schematic diagram of a experimental protocol and mouse model used to test the in vivo effects of human bait CAR T cells.

FIG. 29B are a series of BLI images showing leukemia in mice treated according to the experimental protocol of FIG. 29A at Day-2, Day 2, Day 6, Day 9) and Day 13.

FIG. 29C is a graph showing the results of the mean flux of leukemia (quantified by BLI imaging) in mice treated according to the experimental protocol of FIG. 29A at Day-2, Day 2, Day 6, Day 9) and Day 13.

### DETAILED DESCRIPTION OF THE INVENTION

The present invention generally provides cells, including immune cells (e.g., T cells, B cells, Natural Killer (NK) cells, monocytes, macrophages or artificially generated cells with immune effector function) derived from a patient, a healthy donor, a differentiated stem cell (including but not limited to induced pluripotent stem cells (iPSC), embryonic stem cells, hematopoietic and/or other tissue specific stem cells) or a non-human source, which are genetically modified to express a chimeric antigen receptor (CAR) that specifically binds CD8+ T cells, and methods of use thereof for the treatment of autoimmune disease, T cell leukemia, solid organ transplant rejection, or any disease involving pathologic T cells.

The present invention provides a immune cell (e.g. T cell) expressing a chimeric antigen receptor (CAR) comprising: (a) an ectodomain comprising i. a beta-2 microglobulin leader peptide, ii. a cognate peptide that is recognized by a CD8+ T-cell Receptor, iii. at least one linker domain, iv. a beta-2 microglobulin peptide, v. a MHC class I (MHCI), a HLA-A, a HLA-B or a HLA-C domain; and vi. a stalk/hinge domain: (b) a transmembrane domain: (c) at least one costimulatory domain; and (d) an intracellular signaling domain.

The present disclosure overcomes problems associated with current technologies by providing immune cells (e.g. T cells) such as for the treatment of immune related diseases such as autoimmune disease, T cell leukemia, chronic inflammatory disease and solid transplant rejection. Pathologic T cell reactivity is a component of many diseases, including autoimmune diseases. The present disclosure represents the first discovery and the first use of immune cells (e.g. T cells) expressing chimeric antigen receptors to target other T cells, in particular CD8+ T cells and/or pathologic T cells. The present disclosure is based, at least in part, on the discovery that immune cell (e.g. T-cell) activation mediated by engagement of 1) the ectodomain of a CAR that comprises a MHCI bound cognate antigen with 2) the T cell receptor of a T cell (e.g. CD8+ T cell or pathologic T cell) that specifically binds to the cognate antigen and/or MHC molecule, leads to selective elimination of the T cell that binds to the cognate antigen. Accordingly, the present disclosure provides immune cells expressing CARs that specifically bind CD8+ T cells and/or pathologic T cells, and methods of generating the cells and methods of using this population of cells.

Genetic reprogramming of immune cells (e.g. T cells), for adoptive cancer immunotherapy has clinically relevant applications and benefits such as 1) increased ability to recognize target cells 2) increased cell persistence and proliferation. Accordingly, the present disclosure also provides methods for treating immune-related disorders, such as autoimmune disease, comprising adoptive cell immunotherapy with any of the engineered immune cells provided herein.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein in the specification, “a” or “an” may mean one or more. As used herein in the claim(s), when used in conjunction with the word “comprising,” the words “a” or “an” may mean one or more than one.

As used herein, the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

As used herein, the term “about” is used to indicate that a value includes the inherent variation of error for the device, the method being employed to determine the value, or the variation that exists among the study subjects.

As used herein, the term “portion” when used in reference to a polypeptide or a peptide refers to a fragment of the polypeptide or peptide. In some embodiments, a “portion” of a polypeptide or peptide retains at least one function and/or activity of the full-length polypeptide or peptide from which it was derived. In some embodiments, if a full-length polypeptide binds a given ligand, a portion of that full-length polypeptide also binds to the same ligand.

The terms “protein” and “polypeptide” are used interchangeably herein.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means: or in relation to a cell, the term refers to a cell that was isolated and subsequently introduced into a cell population or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature. The term “exogenous” is used interchangeably with the term “heterologous”.

By “expression construct” or “expression cassette” is used to mean a nucleic acid molecule that is capable of directing transcription. An expression construct includes, at a minimum, one or more transcriptional control elements (such as promoters, enhancers or a structure functionally equivalent thereof) that direct gene expression in one or more desired cell types, tissues or organs. Additional elements, such as a transcription termination signal, may also be included.

A “vector” or “construct” (sometimes referred to as a gene delivery system or gene transfer “vehicle”) refers to a macromolecule or complex of molecules comprising a polynucleotide, or the protein expressed by said polynucleotide, to be delivered to a host cell, either in vitro or in vivo.

A “plasmid.” a common type of a vector, is an extra-chromosomal DNA molecule separate from the chromosomal DNA that is capable of replicating independently of the chromosomal DNA. In certain cases, it is circular and double-stranded.

An “origin of replication” (“ori”) or “replication origin” is a DNA sequence, that when present in a plasmid in a cell is capable of maintaining linked sequences in the plasmid and/or a site at or near where DNA synthesis initiates. As an example, an ori for EBV (Ebstein-Barr virus) includes FR sequences (20 imperfect copies of a 30 bp repeat), and preferably DS sequences: however, other sites in EBV bind EBNA-1, e.g., Rep* sequences can substitute for DS as an origin of replication (Kirshmaier and Sugden. 1998). Thus, a replication origin of EBV includes FR. DS or Rep* sequences or any functionally equivalent sequences through nucleic acid modifications or synthetic combination derived therefrom. For example, methods of the present disclosure may also use genetically engineered replication origin of EBV, such as by insertion or mutation of individual elements.

A “gene,” “polynucleotide,” “coding region,” “sequence,” “segment,” “fragment,” or “transgene” that “encodes” a particular protein. is a section of a nucleic acid molecule that is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to. cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “control elements” refers collectively to promoter regions, polyadenylation signals, transcription termination sequences, upstream regulatory domains, origins of replication, internal ribosome entry sites (IRES), enhancers, splice junctions, and the like, which collectively provide for the replication, transcription, post-transcriptional processing, and translation of a coding sequence in a recipient cell. Not all of these control elements need be present so long as the selected coding sequence is capable of being replicated, transcribed, and translated in an appropriate host cell.

The term “promoter” is used herein to refer to a nucleotide region comprising a DNA regulatory sequence, wherein the regulatory sequence is derived from a gene that is capable of binding to a RNA polymerase and allowing for the initiation of transcription of a downstream (3′ direction) coding sequence. It may contain genetic elements at which regulatory proteins and molecules may bind, such as RNA polymerase and other transcription factors, to initiate the specific transcription of a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

By “enhancer” is meant a nucleic acid sequence that, when positioned proximate to a promoter, confers increased transcription activity relative to the transcription activity resulting from the promoter in the absence of the enhancer domain.

By “operably linked” with reference to nucleic acid molecules is meant that two or more nucleic acid molecules (e.g., a nucleic acid molecule to be transcribed, a promoter, and an functional effector element) are connected in such a way as to permit transcription of the nucleic acid molecule. “Operably linked” with reference to peptide and/or polypeptide molecules means that two or more peptide and/or polypeptide molecules are connected in such a way as to yield a single polypeptide chain, i.e., a fusion polypeptide, having at least one property of each peptide and/or polypeptide component of the fusion. The fusion polypeptide is preferably chimeric, i.e., composed of molecules that are not found in a single polypeptide in nature.

The term “homology” refers to the percent of identity between the nucleic acid residues of two polynucleotides or the amino acid residues of two polypeptides. The correspondence between one sequence and another can be determined by techniques known in the art. For example, homology can be determined by a direct comparison of the sequence information between two polypeptides by aligning the sequence information and using readily available computer programs. Alternatively, homology can be determined by hybridization of polynucleotides under conditions that promote the formation of stable duplexes between homologous regions, followed by digestion with single strand-specific nuclease(s), and size determination of the digested fragments. Two polynucleotide (e.g., DNA), or two polypeptide, sequences are “substantially homologous” to each other when at least about 80%, at least about 90%, and most preferably at least about 95% of the nucleotides, or amino acids, respectively match over a defined length of the molecules, as determined using the methods above.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

As used herein, the term “subject” or “subject in need thereof refers to a mammal, preferably a human being, male or female at any age that is in need of a therapeutic intervention, a cell transplantation or a tissue transplantation. Typically, the subject is in need of therapeutic intervention, cell or tissue transplantation (also referred to herein as recipient) due to a disorder or a pathological or undesired condition, state, or syndrome, or a physical, morphological or physiological abnormality which is amenable to treatment via therapeutic intervention, cell or tissue transplantation.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a NK cell, B cell, or a T cell, or innate immune cell to a stimulus. In one embodiment, the response is specific for a particular antigen (an “antigen-specific response”).

As used herein, the term “antigen” is a molecule capable of being bound by an antibody, T-cell receptor, Chimeric Antigen Receptor and or engineered immune receptor. An antigen may generally be used to induce a humoral immune response and/or a cellular immune response leading to the production of B and/or T lymphocytes.

The terms “tumor-associated antigen,” “tumor antigen” and “cancer cell antigen” are used interchangeably herein. In each case, the terms refer to proteins, glycoproteins or carbohydrates that are specifically or preferentially expressed by cancer cells.

An “epitope” is the site on an antigen recognized by an antibody as determined by the specificity of the amino acid sequence. Two antibodies are said to bind to the same epitope if each competitively inhibits (blocks) binding of the other to the antigen as measured in a competitive binding assay. Alternatively, two antibodies bind to the same epitope if most amino acid mutations in the antigen that reduce or eliminate binding of one antibody reduce or eliminate binding of the other. Two antibodies are said to have overlapping epitopes if each partially inhibits binding of the other to the antigen, and/or if some amino acid mutations that reduce or eliminate binding of one antibody reduce or eliminate binding of the other.

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the micro-organisms (known as commensal organisms) that normally colonize mucosal surfaces, or may be a hybrid constituted from separate other peptides.

A “parameter of an immune response” is any particular measurable aspect of an immune response, including, but not limited to, cytokine secretion (IFN-γ, etc.), chemokine secretion, altered migration or cell accumulation, immunoglobulin production, dendritic cell maturation, regulatory activity, number of immune cells and proliferation of any cell of the immune system. Another parameter of an immune response is structural damage or functional deterioration of any organ resulting from immunological attack. One of skill in the art can readily determine an increase in any one of these parameters, using known laboratory assays. In one specific non-limiting example, to assess cell proliferation, incorporation of 3H-thymidine can be assessed. A “substantial” increase in a parameter of the immune response is a significant increase in this parameter as compared to a control. Specific, non-limiting examples of a substantial increase are at least about a 50% increase, at least about a 75% increase, at least about a 90% increase, at least about a 100% increase, at least about a 200% increase, at least about a 300% increase, and at least about a 500% increase. Similarly, an inhibition or decrease in a parameter of the immune response is a significant decrease in this parameter as compared to a control. Specific, non-limiting examples of a substantial decrease are at least about a 50% decrease, at least about a 75% decrease, at least about a 90% decrease, at least about a 100% decrease, at least about a 200% decrease, at least about a 300% decrease, and at least about a 500% decrease. A statistical test, such as a non-parametric ANOVA, or a T-test, can be used to compare differences in the magnitude of the response induced by one agent as compared to the percent of samples that respond using a second agent. In some examples, p≤0.05 is significant, and indicates that the chance that an increase or decrease in any observed parameter is due to random variation is less than 5%. One of skill in the art can readily identify other statistical assays of use.

“Treating” or treatment of a disease or condition refers to executing a protocol or treatment plan, which may include administering one or more drugs to a patient, in an effort to alleviate signs or symptoms of the disease or the recurrence of the disease. Desirable effects of treatment include decreasing the rate of disease progression, ameliorating or palliating the disease state, and remission, increased survival, improved quality of life or improved prognosis. Alleviation or prevention can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and includes protocols or treatment plans that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis or recurrence. Treatment of cancer may also refer to prolonging survival of a subject with cancer.

“Antigen recognition moiety” or “antigen recognition domain” refers to a molecule or portion of a molecule that specifically binds to an antigen. In one embodiment, the antigen recognition moiety is an antibody, antibody like molecule or fragment thereof and the antigen is a tumor antigen.

“Antibody” as used herein refers to monoclonal or polyclonal antibodies. The term “monoclonal antibodies,” as used herein, refers to antibodies that are produced by a single clone of B-cells and bind to the same epitope. In contrast, “polyclonal antibodies” refer to a population of antibodies that are produced by different B-cells and bind to different epitopes of the same antigen. A whole antibody typically consists of four polypeptides: two identical copies of a heavy (H) chain polypeptide and two identical copies of a light (L) chain polypeptide. Each of the heavy chains contains one N-terminal variable (VH) region and three C-terminal constant (CHL CH2 and CH3) regions, and each light chain contains one N-terminal variable (VL) region and one C-terminal constant (CL) region. The variable regions of each pair of light and heavy chains form the antigen binding site of an antibody. The VH and VL regions have a similar general structure, with each region comprising four framework regions, whose sequences are relatively conserved. The framework regions are connected by three complementarity determining regions (CDRs). The three CDRs, known as CDR1, CDR2, and CDR3, form the “hypervariable region” of an antibody, which is responsible for antigen binding.

“Antibody like molecules” may be for example proteins that are members of the Ig-superfamily which are able to selectively bind a partner.

The terms “fragment of an antibody.” “antibody fragment.”, “functional fragment of an antibody.” and “antigen-binding portion” are used interchangeably herein to mean one or more fragments or portions of an antibody that retain the ability to specifically bind to an antigen (see, generally, Holliger et al. (2005) Nat. Biotech. 23 (9): 1126-29). The antibody fragment desirably comprises, for example, one or more CDRs, the variable region (or portions thereof), the constant region (or portions thereof), or combinations thereof.

Examples of antibody fragments include, but are not limited to, (i) a Fab fragment, which is a monovalent fragment consisting of the VL, VH, CL, and CHI domains: (ii) a F (ab′) 2 fragment, which is a bivalent fragment comprising two Fab fragments linked by a disulfide bridge at the stalk region: (iii) a Fv fragment consisting of the VL and VH domains of a single arm of an antibody: (iv) a single chain Fv (scFv), which is a monovalent molecule consisting of the two domains of the Fv fragment (i.e., VL and VH) joined by a synthetic linker which enables the two domains to be synthesized as a single polypeptide chain (see, e.g., Bird et al. (1988), Science 242:423-6; Huston et al. (1988) Proc. Natl. Acad. Sci. USA 85:5879-83; and Osbourn et al. (1998) Nat. Biotechnol. 16:778-81) and (v) a diabody, which is a dimer of polypeptide chains, wherein each polypeptide chain comprises a VH connected to a VL by a peptide linker that is too short to allow pairing between the VH and VL on the same polypeptide chain, thereby driving the pairing between the complementary domains on different VH-VL polypeptide chains to generate a dimeric molecule having two functional antigen binding sites. Antibody fragments are known in the art and are described in more detail in, e.g., U.S. Patent Application Publication 2009/0093024 A1.

A “chimeric antigen receptor” is also known as an artificial cell receptor, a chimeric cell receptor, or a chimeric immunoreceptor. Chimeric antigen receptors (CARs) are engineered receptors, which graft a selected specificity onto an immune effector cell. CARs typically have an extracellular domain (ectodomain), a transmembrane domain and an intracellular (endodomain) domain. In some embodiments, the ectodomain comprises which an antigen-binding domain and a stalk region. In some embodiments, the ectodomain comprises i. a beta-2 microglobulin leader peptide (B2ML), ii. a cognate peptide that is recognized by a CD8+ T-cell Receptor (P), iii. at least one linker domain (L), iv. a beta-2 microglobulin peptide (B2M), andv. a MHC class I (MHCI), a HLA-A, a HLA-B or a HLA-C domain.

A “stalk region”, which encompasses the terms “spacer region” or “hinge domain” or “hinge”, is used to link the antigen-binding domain to the transmembrane domain. As used herein, the term “stalk region” generally means any oligonucleotide or polypeptide that functions to link the transmembrane domain to, either the extracellular domain or, the cytoplasmic domain in the polypeptide chain of a CAR. In embodiments, it is flexible enough to allow the antigen-binding domain to orient in different directions to facilitate antigen recognition.

The term “functional portion,” when used in reference to a CAR, refers to any part or fragment of a CAR described herein, which part or fragment retains the biological activity of the CAR of which it is a part (the parent CAR). In reference to a nucleic acid sequence encoding the parent CAR, a nucleic acid sequence encoding a functional portion of the CAR can encode a protein comprising, for example, about 10%, 25%, 30%, 50%, 68%, 80%, 90%, 95%, or more, of the parent CAR.

The phrases “pharmaceutical or pharmacologically acceptable” refers to molecular entities and compositions that do not produce an adverse, allergic, or other untoward reaction when administered to an animal, such as a human, as appropriate. For animal (e.g., human) administration, it will be understood that preparations should meet sterility, pyrogenicity, general safety, and purity standards as required, e.g., by the FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “T cell” refers to T lymphocytes, and includes, but is not limited to, γ/δ T cells, α/β T cells, NK T cells, CD4⁺ T cells and CD8⁺ T cells. CD4⁺ T cells include THO, Tₕ1 and TH2 cells, as well as regulatory T cells (T_{reg}). There are at least three types of regulatory T cells: CD4⁺ CD25⁺ T_{reg}, CD25 T_{H}3 T_{reg}, and CD25 T_{R} I T_{reg}. “Cytotoxic T cell” refers to a T cell that can kill another cell. The majority of cytotoxic T cells are CD8⁺ MHC class I-restricted T cells, however some cytotoxic T cells are CD4⁺. In some embodiments, the T cell of the present disclosure is CD4⁺ or CD8⁺.

The term “pathologic T cell” refers to a T cell that is involved or has the potential to be involved in an autoimmune response or disorder. Pathologic T cells are ones which cause autologous host cells or tissues, orthotopically transplanted cells or tissues, or the combination of these cells or tissues with foreign antigens.

“Tumor antigen” as used herein refers to any antigenic substance produced, expressed or overexpressed in tumor cells. It may, for example, trigger an immune response in the host.

The term “antigen presenting cells (APCs)” refers to a class of cells capable of presenting one or more antigens in the form of peptide-MHC complex recognizable by specific effector cells of the immune system, and thereby inducing an effective cellular immune response against the antigen or antigens being presented. APCs can be intact whole cells such as macrophages, B cells, endothelial cells, activated T cells, and dendritic cells: or other molecules, naturally occurring or synthetic, such as purified MHC Class I molecules complexed to 2-microglobulin.

The term “culturing” refers to the in vitro maintenance, differentiation, and/or propagation of cells in suitable media. By “enriched” is meant a composition comprising cells present in a greater percentage of total cells than is found in the tissues where they are present in an organism.

### II. Immune Cells

Certain embodiments of the present disclosure concern immune cells which express a chimeric antigen receptor (CAR). The immune cells may be T cells (e.g., regulatory T cells, CD4⁺ T cells, CD8⁺ T cells, or gamma-delta T cells), NK cells, invariant NK cells, NKT cells, stem cells (e.g., mesenchymal stem cells (MSCs) or induced pluripotent stem (iPSC) cells). In some embodiments, the cells are monocytes or granulocytes, e.g., myeloid cells, macrophages, neutrophils, dendritic cells, mast cells, eosinophils, and/or basophils. Also provided herein are methods of producing and engineering the immune cells and methods of using and administering the cells for adoptive cell therapy, in which case the cells may be autologous or allogeneic. Thus, the immune cells may be used as immunotherapy, such as to target cancer cells.

The immune cells may be isolated from subjects, particularly human subjects. The immune cells can be obtained from a subject of interest, such as a subject suspected of having a particular disease or condition, a subject suspected of having a predisposition to a particular disease or condition, or a subject who is undergoing therapy for a particular disease or condition. Thus, the cells will be autologous to the subject in need of therapy. Alternatively, the immune cells can be obtained from a donor and therefore be allogeneic to the subject in need of therapy.

When the population of immune cells is obtained from a donor distinct from the subject, the donor is preferably allogeneic, provided the cells obtained are subject-compatible in that they can be introduced into the subject. Allogeneic donor cells are may or may not be human leukocyte antigen (HLA)-compatible. To be rendered subject-compatible, allogeneic cells can be treated to reduce immunogenicity.

### 1. T Cells

T-cells play a major role in cell-mediated-immunity (no antibody involvement). Its T-cell receptors (TCR) differentiate themselves from other lymphocyte types. The thymus, a specialized organ of the immune system, is primarily responsible for the T cell's maturation. There are six types of T-cells, namely: Helper T-cells (e.g CD4+ cells). Cytotoxic T-cells (also known as TC, cytotoxic T lymphocyte. CTL. T-killer cell, cytolytic T cell. CD8+ T-cells or killer T cell). Memory T-cells ((i) stem memory TSCM cells, like naive cells, are CD45RO−. CCR7+. CD45RA+. CD62L+ (L-selectin). CD27+. CD28+ and IL-7Ra+. but they also express large amounts of CD95. IL-2R. CXCR3, and LFA-1, and show numerous functional attributes distinctive of memory cells): (ii) central memory TCM cells express L-selectin and the CCR7, they secrete IL-2, but not IFNg or IL-4, and (iii) effector memory TEM cells, however, do not express L-selectin or CCR7 but produce effector cytokines like IFNg and IL-4). Regulatory T-cells (Tregs, suppressor T cells, or CD4+CD25+ regulatory T cells), Natural Killer T-cells (NKT) and Gamma Delta T-cells.

The T cells of the immunotherapy can come from any source known in the art. For example, T cells can be differentiated in vitro from a hematopoietic stem cell population, or T cells can be obtained from a subject. T cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. Additional methods of isolating T cells for a T cell therapy are disclosed in U.S. Patent Publication No. 2013/0287748, which is herein incorporated by references in its entirety.

### 2. Genetically Engineered Chimeric Antigen Receptors

The immune cells of the disclosure (e.g., autologous or allogeneic T cells (e.g., regulatory T cells, CD4⁺ T cells, CD8⁺ T cells, or gamma-delta T cells), NK cells, invariant NK cells, NKT cells, stem cells (e.g., MSCs or iPS cells) can be genetically engineered to express antigen receptors such as engineered CARs. In particular embodiments, T cells are engineered to express a CAR. Multiple CARs, may be added to a single cell type, such as T cells.

In some embodiments, the cells comprise one or more nucleic acids introduced via genetic engineering that encode one or more antigen receptors, and genetically engineered products of such nucleic acids. In some embodiments, the nucleic acids are heterologous. In some embodiments, the nucleic acids are not naturally occurring, such as a nucleic acid not found in nature (e.g., chimeric).

In some embodiments the CAR contains an immune recognition molecule (e.g. MHC class I molecules, MHC class I co-receptors, MHC class II molecules, MHC class II co-receptors, HLA class I molecules, or HLA class II molecules) that is bound to a cognate peptide. In some embodiments, the CAR contains an extracellular antigen-recognition domain that specifically binds to an antigen (e.g., on the surface of a CD8+ T cell or pathologic T cell). In some embodiments, the antigen is a protein expressed on the surface of cells (e.g., on the surface of a CD8+ T cell or a pathologic T cell).

Exemplary engineered antigen receptors, including CARs and recombinant TCRs, as well as methods for engineering and introducing the receptors into cells, include those described, for example, in PCT Publication Nos. WO 2000/14257, WO 2013126726, WO 2012/129514, WO 2014/031687, WO 2013/166321, WO 2013/071154, and WO 2013/123061, U.S. Patent Application Publication Nos. US 2002/131960, US 2013/287748, and US 2013/0149337; and U.S. Pat. Nos. 6,451,995, 7,446,190, 8,252,592, 8,339,645, 8,398,282, 7,446,179, 6,410,319, 7,070,995, 7,265,209, 7,354,762, 7,446,190, 7,446,191, 8,324,353, and 8,479, 118: International Patent Application Publication No.: WO 2014/055668 A1, and European Patent Application Publication No. EP2537416; and/or those described by Sadelain et al., 2013; Davila et al., 2013; Turtle et al., 2012: Wu et al., 2012.

### 3. Chimeric Antigen Receptors

In some aspects, the present disclosure provides a population of genetically modified immune cells (e.g. T cells) engineered to express a chimeric antigen receptor (CAR) and/or a polynucleotide encoding a CAR, wherein the CAR comprises (a) an ectodomain comprising i. a beta-2 microglobulin leader peptide (B2ML), ii. a cognate peptide that is recognized by a CD8+ T-cell Receptor (P), iii. at least one linker domain (L), iv. a beta-2 microglobulin peptide (B2M), and v. a MHC class I (MHCI), a HLA-A, a HLA-B or a HLA-C: (b) a transmembrane domain: (c) at least one costimulatory domain; and (d) an intracellular signaling domain.

In some embodiments, the genetically engineered cells include additional CARS, including activating or stimulatory CARs, co-stimulatory CARs (see, e.g., PCT Publ. No. WO 2014/055668), and/or inhibitory CARs (iCARs, see, e.g., Fedorov et al., 2013). The CARs generally include an extracellular antigen (or ligand) recognition domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone. For example, once an antigen is recognized by the ectodomain, the intracellular signaling components transmit an activation signal to the T cell that induces the T cell to destroy a targeted tumor cell.

### A. Immune Recognition Binding Molecule (Ectodomain)

In some embodiments, the CAR has a ectodomain corresponding to an immune recognition binding molecule that is bound to a cognate peptide. In some embodiments, the CAR has a ectodomain corresponding to an immune recognition binding molecule that is covalently linked to a cognate peptide. In some embodiments, the ectodomain of the CAR comprises i. a beta-2 microglobulin leader peptide (B2ML), ii. a cognate peptide that is recognized by a CD8+ T-cell Receptor (P), iii. at least one linker domain (L), iv. a beta-2 microglobulin peptide (B2M), and v. a MHC class I (MHCI), a HLA-A, a HLA-B or a HLA-C domain.

### MHC/HLA Domains

Exemplary immune recognition binding molecules include but are not limited to MHC class I molecules, MHC class I co-receptors, MHC class II molecules, MHC class II co-receptors, HLA class I molecules, or HLA class II molecules. Endogenously, these proteins are capable of binding peptides resulting from the proteolytic cleavage of protein antigens and representing potential T-cell epitopes, transporting them to the cell surface and presenting them there to specific cells, in particular cytotoxic T-lymphocytes or T-helper cells. The MHC in the genome comprises the genetic region whose gene products expressed on the cell surface are important for binding and presenting endogenous and/or foreign antigens and thus for regulating immunological processes.

The major histocompatibility complex (MHC), also known as human leukocyte antigen (HLA) in humans, is a cell surface multi-component molecule found in all vertebrates that mediates interactions of leukocytes with other leukocytes or other cells. The MHC gene family is divided into three groups: class I, class II and class III. In some embodiments, the CAR comprises a MHCI molecule. The heavy chain of the MHC molecules is of class I, and the light chain is β-2 microglobulin. In some embodiments, the beta 2 microglobulin comprises a beta-2 microglobulin leader peptide (B2ML) and a beta-2 microglobulin peptide (B2M).

In some embodiments, the “beta-2 microglobulin leader peptide” (B2ML) of a CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 1.

In some embodiments, the “beta-2 microglobulin peptide” (B2M) of a CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 2.

In some embodiments, the “MHC-I domain” “MHC-I domain-WT” or “H-2 KB-WT” of a CAR provided herein may comprise or consist of a mouse MHCI domain comprising an amino acid sequence having at least 90%, 91%, 92%, 93%, 94%, 95%, 96%, 97%, 98%, 99% or 100% identity with the amino acid sequence of SEQ ID NO: 3.

In humans, MHC is referred to as human leukocyte antigen (HLA). The HLA class I (HLA-I) protein is expressed on all nucleated cells and consists of an HLA class I heavy chain (or a chain) and β-2 microglobulin (B2M). HLA class I protein presents peptides on the cell surface to CD8+ cytotoxic T cells. Six HLA class I a chains have been identified to date, including three classical (HLA-A, HLA-B and HLA-C) and three non-classical (HLA-E, HLA-F and HLA-G) a chains. The specificity for peptide binding on the HLA class I molecule peptide binding cleft is determined by the a chain. Recognition by CD8+ T cells of the peptides presented by the HLA class I molecule mediates cellular immunity. In some embodiments, the CAR comprises an HLA-A (UniProt ID No. Q29757), HLA-A2 (UniProt ID No. Q95387), HLA-B (UniProt ID No. P01889) or HLA-C domain (UniProt No: P10321). In some embodiments, the CAR comprises a human β-2 microglobulin (UniProt ID No. P61769).

In some embodiments, the “MHC-I domain-WT HLA-A2” of a CAR provided herein may comprise or consist of a human HLA-A2 domain comprising an amino acid sequence having at least 90%, 91%, 92%, 93%, 94%, 95%, 96%, 97%, 98%, 99% or 100% identity with the amino acid sequence of SEQ ID NO: 68.

In some embodiments, the MHCI/HLA domain comprises a CD8 binding domain for T-cell activation. In some embodiments, the CD3ξ intracellular signaling domain comprises a mutation in an CD8 binding domain. In some embodiments, the mutations provide a MHCI with intermediate CD8 binding function. In some embodiments, the mutations provide a MHCI with null CD8 binding function. In some embodiments the mutations provide decreased T cell activation and prolonged survival. Examples of mutations in CD8 binding domains are provided in Li. et al: Front Immuno/2013:4:383 and Schott, et al: Eur. J. Immunol. 2002 32:3425-3434. In some embodiments, the MHCI domain comprises a D227N mutation. In some embodiments, the MHCI domain comprises a Q226L mutation. In some embodiments, the MHCI domain comprises a D227N and/or a Q226L mutation. In some embodiments, the HLA-A domain comprises a A245V mutation.

In some embodiments, the “MHC-I domain Intermediate CD8 binder” or “H-2 KB from SCT2” of a CAR provided herein may comprise or consist of a mouse MHCI domain comprising an amino acid sequence having at least 90%, 91%, 92%, 93%, 94%, 95%, 96%, 97%, 98%, 99% or 100% identity with the amino acid sequence of SEQ ID NO: 4.

In some embodiments, the “MHC-I domain Null CD8 binder” of a CAR provided herein may comprise or consist of a mouse MHCI domain comprising an amino acid sequence having at least 90%, 91%, 92%, 93%, 94%, 95%, 96%, 97%, 98%, 99% or 100% identity with the amino acid sequence of SEQ ID NO: 5.

In some embodiments, the “MHC-I domain-A245V HLA-A2 CD8 null binder” of a CAR provided herein may comprise or consist of a human HLA-A2 domain comprising an amino acid sequence having at least 90%, 91%, 92%, 93%, 94%, 95%, 96%, 97%, 98%, 99% or 100% identity with the amino acid sequence of SEQ ID NO: 69.

### Cognate Peptide

MHC class I molecules (HLA class I in human) consist of a heavy chain and a light chain and are capable of binding a short peptide with suitable binding motifs, and presenting it to cytotoxic T-lymphocytes (i.e. cognate peptide). Endogenously, the cognate peptide bound by the MHC molecules of class I originates from an endogenous protein antigen.

Any suitable cognate antigen may find use in the present method. Exemplary target antigens include, but are not limited to, an autoantigen of an autoimmune disease or a foreign antigen that mimics an autoantigen in eliciting autoimmune response. Exemplary target antigens include, but are not limited to ovalbumin (UniProt ID No. P01012) and NYESO-1 (UniProt ID No. P78358). In some embodiments, the cognate peptide is isolated or derived from ovalbumin.

In some embodiments, the cognate peptide comprises ovalbumin residues 257-264 (SEQ ID NO: 6). In some embodiments the cognate peptide comprises a mutation that affects (target) OT-I activation without affecting binding activity. Examples of mutations of ovalbumin cognate peptide residues are provided in Bevans, et al: Nature. 2009, 458 (7235): 211-214. In some embodiments, the cognate peptide comprises the amino acid sequence of SX₁X₂X₃FEKL (SEQ ID NO: 62) wherein X₁ is A or I: X₂ is I or Y; and X₃ is N, Q. T or V. In some embodiments, the cognate peptide comprises the amino acid sequence of SEQ ID NOs: 6-10, or 56.

In some embodiments, the cognate peptide compresses a NY-ESO peptide residues 157-165 (SEQ ID NO: 66).

### Linker Domains

In some embodiments, the MHCI or HLA domain, cognate peptide and β-2 microglobulin are covalently linked through at least one linker (L) domain. Examples of covalently linked MHCI, cognate peptide and B2M domains are provided in Yu, et al. J Immunol 2002:168:3145-3149. In some embodiments, the B2M, cognate peptide and MHC1/HLA domains are directionally linked, for example, from N- to C-terminus.

In some embodiments, the “linker” of the ectodomain of a CAR provided herein may comprise or consist of the amino acid sequence of SEQ ID NO: 11.

In some embodiments, the ectodomain of the CAR comprises the following in the N-terminal to C-terminal direction: N-term-B2ML-P-(L)ₓ-B2M-(L)_{y}-(MHCI/HLA-A/HLA-B/HLA-C)-stalk/hinge-C-term, wherein x is any integer between 0-10; and wherein y is any integer between 0-10. In some embodiments, x is any integer between 0-5 and y is any integer between 0-5.

In some embodiments, x is 0 and y is 1. In some embodiments, x is 0 and y is 2. In some embodiments, x is 0 and y is 3. In some embodiments, x is 0 and y is 4. In some embodiments, x is 0 and y is 5. In some embodiments, x is 1 and y is 1. In some embodiments, x is 1 and y is 2. In some embodiments, x is 1 and y is 3. In some embodiments, x is 1 and y is 4. In some embodiments, x is 1 and y is 5. In some embodiments, x is 2 and y is 1. In some embodiments, x is 2 and y is 2. In some embodiments, x is 2 and y is 3. In some embodiments, x is 2 and y is 4. In some embodiments, x is 2 and y is 5. In some embodiments, x is 3 and y is 1. In some embodiments, x is 3 and y is 2. In some embodiments, x is 3 and y is 3. In some embodiments, x is 3 and y is 4. In some embodiments, x is 3 and y is 5. In some embodiments, x is 4 and y is 1. In some embodiments, x is 4 and y is 2. In some embodiments, x is 4 and y is 3. In some embodiments, x is 4 and y is 4. In some embodiments, x is 4 and y is 5. In some embodiments, x is 5 and y is 1. In some embodiments, x is 5 and y is 2. In some embodiments, x is 5 and y is 3. In some embodiments, x is 5 and y is 4. In some embodiments, x is 5 and y is 5.

In some embodiments, the ectodomain of the CAR comprises the following in the N-terminal to C-terminal direction: N-term-B2ML-P-(G4S) 4-B2M-(G4S) 3-(MHCI-WT)-stalk/hinge-C′-term.

In some embodiments, the CAR comprises the following in the N-terminal to C-terminal direction: N-term-B2ML-P-(Linker 1)ₓ-(Linker 2)_{z}-B2M-(Linker 2) y-(HLA-A/HLA-B/HLA-C), wherein x is any integer between 0-5: wherein y is any integer between 0-5; and wherein z is any integer between 0-5.

In some embodiments, the CAR comprises the following in the N-terminal to C-terminal direction: N-term-B2ML-P-(Linker 1)₁-(Linker 2)₂-B2M-(Linker 2)₃-(HLA-A/HLA-B/HLA-C). In some embodiments, Linker 1 comprises the amino acid sequence of SEQ ID NO: 86. In some embodiments, Linker 2 comprises the amino acid sequence of SEQ ID NO: 11.

### B. Signal Peptides

In some embodiments, any of the CARs provided herein comprises a signal peptide (also known as a signal peptide, signal sequence, signal peptide sequence, leader peptide, and leader peptide sequence). In some embodiments, the antigen recognition domain of the CAR described herein comprises a signal peptide or a leader peptide sequence. Exemplary signal sequences include but are not limited to a CD8a signal sequence or an IgG signal sequence. In some embodiments, the CAR described herein does not comprise a signal peptide. In some embodiments, the T cell or populations of T cells provided herein comprise a CAR comprising a signal peptide. In some embodiments, the T cell or populations of T cell provided herein comprise a CAR that does not comprise a signal peptide.

### C. Hinge Domains

In some embodiments, a hinge domain (also known as a spacer region or a stalk region) is located between the antigen recognition domain and the transmembrane domain of the CAR. In particular, stalk regions are used to provide more flexibility and accessibility for the extracellular antigen recognition domain. In some embodiments, a hinge domain may comprise up to about 300 amino acids. In some embodiments, the hinge comprises about 10 to about 100 amino acids in length. In some embodiments, the hinge comprises about 25 to about 50) amino acids in length. In some embodiments, the hinge domain establishes an optimal effector-target inter membrane distance. In some embodiments, the hinge domain provides flexibility for antigen recognition domain to bind the target antigen. Any protein that is stable and/or dimerizes can serve this purpose. In some embodiments, the T cell or populations of T cells provided herein comprise a CAR comprising a hinge domain. In some embodiments, the T cell or populations of T cell provided herein comprise a CAR that does not comprise a hinge domain.

A hinge domain may be derived from all or part of naturally occurring molecules, such as from all or part of the extracellular region of CD8, CD8a, CD4, CD28, 4-1BB, or IgG (in particular, the hinge domain of an IgG, for example from IgG1, IgG2 or IgG4), or from all or part of an antibody heavy-chain constant region. Alternatively, the hinge domain may be a synthetic sequence that corresponds to a naturally occurring hinge sequence, or may be an entirely synthetic hinge sequence.

In some embodiments, the stalk domain of a CAR provided herein may comprise or consist of a mouse CD28 stalk domain comprising an amino acid sequence having at least 90%, 91%, 92%, 93%, 94%, 95%, 96%, 97%, 98%, 99% or 100% identity with the amino acid sequence of SEQ ID NO: 63.

In some embodiments, the stalk domain of a CAR provided herein may comprise or consist of a human CD28 stalk domain comprising an amino acid sequence having at least 90%, 91%, 92%, 93%, 94%, 95%, 96%, 97%, 98%, 99% or 100% identity with the amino acid sequence of SEQ ID NO: 71.

### D. Transmembrane Domains

Suitable transmembrane domains for a CAR disclosed herein have the ability to (a) be expressed at the surface of a cell, which is in some embodiments an immune cell (e.g. a T cell), and/or (b) interact with the ectodomain and intracellular domain for directing cellular response of an immune cell against a predefined target cell. The transmembrane domain can be derived either from a natural or from a synthetic source. The transmembrane domain can be derived from any membrane-bound or transmembrane protein. As non-limiting examples, the transmembrane domains can include the transmembrane region(s) of alpha, beta, delta, or gamma of the T-cell receptor: or a transmembrane region from CD28, CD8, CD8a, CD8 beta, CD3-epsilon, CD3-delta, CD3-gamma, CD3z, CD4, 4-1BB, OX40, ICOS, PD-1, LAG-3, 2B4 or BTLA transmembrane domain or a portion of any of the foregoing or a combination of any of the foregoing. In some embodiments, the transmembrane domain comprises a CD28 transmembrane domain.

Alternatively, the transmembrane domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the intracellular domain of a CAR. In some embodiments, the linker is a glycine-serine linker.

In some embodiments, the transmembrane domain of a CAR provided herein may comprise or consist of a mouse CD28 transmembrane domain comprising an amino acid sequence having at least 90%, 91%, 92%, 93%, 94%, 95%, 96%, 97%, 98%, 99% or 100% identity with the amino acid sequence of SEQ ID NO: 12.

In some embodiments, the transmembrane domain of a CAR provided herein may comprise or consist of a human CD28 transmembrane domain comprising an amino acid sequence having at least 90%, 91%, 92%, 93%, 94%, 95%, 96%, 97%, 98%, 99% or 100% identity with the amino acid sequence of SEQ ID NO: 13.

### E. Costimulatory Domains

The intracellular domain of a CAR provided herein may comprise one or more costimulatory domains. Exemplary costimulatory domains include, but are not limited to a CD28, 4-1BB (CD137), CD97, CD11a-CD18, CD2, ICOS, CD27, CD154, CD8a, OX40 (CD134) costimulatory domain, or a fragment thereof, or a combination thereof. In some embodiments, a CAR described herein comprises a CD28 costimulatory domain or a fragment thereof.

In some embodiments, the costimulatory domain of a CAR provided herein may comprise or consist of a mouse CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 14.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 15.

In some embodiments, the costimulatory domain of a CAR provided herein may comprise or consist of a human CD28 costimulatory domain comprising an amino acid sequence having at least 90%, 91%, 92%, 93%, 94%, 95%, 96%, 97%, 98%, 99% or 100% identity with the amino acid sequence of SEQ ID NO: 35.

### F. Intracellular Signaling Domain

In some embodiments, the intracellular signaling domain of a CAR disclosed herein is responsible for activation of at least one of the normal effector functions of the immune cell (e.g. T cell) in which the CAR is expressed. The terms “intracellular domain” is used to refer to a domain that comprises a co-stimulatory domain and/or an intracellular signaling domain. The term “effector function” refers to a specialized function of a cell. Effector function of a T-cell, for example, may be cytolytic activity or helper activity including the secretion of cytokines. The term “intracellular signaling domain” refers to the portion of a protein which transduces the effector function signal and directs the cell to perform a specialized function. While usually an entire intracellular signaling domain can be employed, in many cases it is not necessary to use the entire chain. To the extent that a truncated portion of the intracellular signaling domain is used, such intracellular signaling domain portion may be used in place of the intact chain as long as it transduces the effector function signal. The term intracellular signaling domain is thus meant to include any truncated portion of the intracellular signaling domain sufficient to transduce the effector function signal.

In some embodiments, the intracellular signaling domain comprises a signaling domain for T-cell activation. In some instances, the intracellular signaling domain for T-cell activation comprises an intracellular domain derived from CD3ξ (also referred to as “CD3zeta” or “CD32”). In some embodiments, the CAR described herein comprises at least one intracellular signaling domain of a CD3ξ or a portion thereof. In some embodiments, the CAR described herein has an intracellular signaling domain comprising a domain derived from CD3ξ.

In some embodiments, the CD3ξ intracellular signaling domain comprises a mutation in an ITAM domain. Examples of mutations in ITAM domains of CD35 are provided in Feucht et al., Nat Med. 2019; 25 (1): 82-88. In some embodiments, each of the two tyrosine residues in one or more of ITAM1, ITAM2, or ITAM3 domains of the CD3ξ intracellular signaling domain are point-mutated to a phenylalanine residue. In some embodiments, the CD3ξ intracellular signaling domain comprises a deletion of one or more of the ITAM1. ITAM2, or ITAM3 domains.

In some embodiments, the activation domain of a CAR described herein may comprise or consist of a human CD3ξ intracellular signaling domain comprising an amino acid sequence having at least 90%, 91%, 92%, 93%, 94%, 95%, 96%, 97%, 98%, 99% or 100% identity with the amino acid sequence of SEQ ID NO: 16.

In some embodiments, the activation domain of a CAR described herein may comprise or consist of a human CD3 intracellular signaling domain comprising an amino acid sequence having at least 90%, 91%, 92%, 93%, 94%, 95%, 96%, 97%, 98%, 99% or 100% identity with the amino acid sequence of SEQ ID NO: 17.

In some embodiments, the activation domain of a CAR described herein may comprise or consist of a human CD3ξ intracellular signaling domain comprising an amino acid sequence having at least 90%, 91%, 92%, 93%, 94%, 95%, 96%, 97%, 98%, 99% or 100% identity with the amino acid sequence of SEQ ID NO: 72.

In some embodiments, the activation domain of a CAR described herein may comprise or consist of a mouse “CD3 (ITAM 2 Live” signaling domain (i.e., a mouse CD3ξ intracellular signaling domain) comprising an amino acid sequence having at least 90%, 91%, 92%, 93%, 94%, 95%, 96%, 97%, 98%, 99% or 100% identity with the amino acid sequence of SEQ ID NO: 18.

In some embodiments, the activation domain of a CAR described herein may comprise or consist of a mouse “CD3ξ Fully Live” intracellular signaling domain (i.e., a mouse CD3ξ intracellular signaling domain) comprising an amino acid sequence having at least 90%, 91%, 92%, 93%, 94%, 95%, 96%, 97%, 98%, 99% or 100% identity with the amino acid sequence of SEQ ID NO: 19.

Included in the scope of the invention are nucleic acid sequences that encode functional portions of the CAR described herein. Functional portions encompass, for example, those parts of a CAR that retain the ability to recognize target cells, or detect, treat, or prevent a disease, to a similar extent, the same extent, or to a higher extent, as the CAR.

In embodiments, the CARs described herein contain additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity of the CAR, as compared to the biological activity of the parent CAR.

The term “functional variant,” as used herein in reference to a CAR, refers to a CAR, a polypeptide, or a protein having substantial or significant sequence identity or similarity to the CAR encoded by a nucleic acid sequence, which functional variant retains the biological activity of the CAR of which it is a variant. Functional variants encompass, for example, those variants of the CAR described herein (the “parent CAR”) that retain the ability to recognize target cells to a similar extent, the same extent, or to a higher extent, as the parent CAR. In reference to a nucleic acid sequence encoding the parent CAR, a nucleic acid sequence encoding a functional variant of the CAR can be for example, about 10% identical, about 25% identical, about 30% identical, about 50% identical, about 65% identical, about 80% identical, about 90% identical, about 95% identical, or about 99% identical to the nucleic acid sequence encoding the parent CAR.

A CAR described herein include (including functional portions and functional variants thereof)glycosylated, amidated, carboxylated, phosphorylated, esterified, N-acylated, cyclized via, e.g., a disulfide bridge, or converted into an acid addition salt and/or optionally dimerized or polymerized.

Table 1 provides exemplary amino acid sequences of the domains which can be used in the CARs described herein. In some embodiments, a CAR provided herein comprises one or more domains described in Table 1, or a fragment or portion thereof.

TABLE 1
Exemplary Amino Acid Sequences of CAR Domains

| | | |
|---|---|---|
| | | SEQ ID |
| Exemplary CAR domains | Amino Acid Sequence | NO: |

B2M Leader

| | | |
|---|---|---|
| Mouse-B2M Leader | MARSVTLVFLVLVSLTGLYA | 1 |
| domain-WT | | |
| Human-B2M Leader | MSRSVALAVLALLSLSGLEA | 65 |
| domain-WT | | |

Cognate Peptide

| | | |
|---|---|---|
| Ovalbumin consensus | SX₁X₂X₃FEKL | 62 |
| peptide | wherein X₁ is A or I; | |
| | X₂ is I or Y; and | |
| | X₃ is N, Q, T or V | |
| Ovalbumin | SIINFEKL | 6 |
| Immunodominant Epitope- | | |
| WT | | |
| (“N4” or “OVA₂₅₇₋₂₆₄”) | | |
| Ovalbumin Peptide Mutant | SIIQFEKL | 8 |
| #1 (“Q4”) | | |
| Ovalbumin Peptide Mutant | SIITFEKL | 9 |
| #2 (“T4”) | | |
| Ovalbumin Peptide Mutant | SIIVFEKL | 10 |
| #3 (“V4”) | | |
| NY-ESO peptide NY157- | SLLMWITQC | 66 |
| 165 WT | | |

B2M

| | | |
|---|---|---|
| Mouse-B2M-1 | IQKTPQIQVYSRHPPENGKPNILNCYVTQFHPPH | 2 |
| | IEIQMLENGKKIPKVEMSDMSFSKDWSFYILAHT | |
| | EFTPTETDTYACRVKHASMAEPKTVYWDRDM | |
| Human-B2M-1 | IQRTPKIQVYSRHPAENGKSNFLNCYVSGFHPSD | 67 |
| | IEVDLLKNGERIEKVEHSDLSFSKDWSFYLLYYT | |
| | EFTPTEKDEYACRVNHVTLSQPKIVKWDRDM | |

MHC-I/HLA domain

| | | |
|---|---|---|
| Mouse-MHC-I domain-WT | GPHSLRYFVTAVSRPGLGEPRYMEVGYVDDTEFV | 3 |
| H-2KB-WT | REDSDAENPRYEPRARWMEQEGPEYWERETQKAK | |
| | GNEQSFRVDLRTLLGYYNQSKGGSHTIQVISGCE | |
| | VGSDGRLLRGYQQYAYDGCDYIALNEDLKTWTAA | |
| | DMAALITKHKWEQAGEAERLRAYLEGTCVEWLRR | |
| | YLKNGNATLLRTDSPKAHVTHHSRPEDKVTLRCW | |
| | ALGFYPADITLTWQLNGEELIQDMELVETRPAGD | |
| | GTFQKWASVVVPLGKEQYYTCHVYHQGLPEPLTL | |
| | RWE | |
| Mouse-MHC-I domain- | GPHSLRYFVTAVSRPGLGEPRYMEVGYVDDTEFV | 4 |
| Intermediate CD8 binder | RFDSDAENPRYEPRARWMEQEGPEYWERETQKAK | |
| H-2KB From SCT2 | GNEQSFRVDLRTLLGYYNQSKGGSHTIQVISGCE | |
| | VGSDGRLLRGYQQYAYDGCDYIALNEDLKTWTAA | |
| | DMAALITKHKWEQAGEAERLRAYLEGTCVEWLRR | |
| | YLKNGNATLLRTDSPKAHVTHHSRPEDKVTLRCW | |
| | ALGFYPADITLTWQLNGEELIQNMELVETRPAGD | |
| | GTFQKWASVVVPLGKEQYYTCHVYHQGLPEPLTL | |
| | RWE | |
| Mouse-MHC-I domain-Null | GPHSLRYFVTAVSRPGLGEPRYMEVGYVDDTEFV | 5 |
| CD8 binder | REDSDAENPRYEPRARWMEQEGPEYWERETQKAK | |
| H-2KB From SCT3 | GNEQSFRVDLRTLLGYYNQSKGGSHTIQVISGCE | |
| | VGSDGRLLRGYQQYAYDGCDYIALNEDLKTWTAA | |
| | DMAALITKHKWEQAGEAERLRAYLEGTCVEWLRR | |
| | YLKNGNATLLRTDSPKAHVTHHSRPEDKVTLRCW | |
| | ALGFYPADITLTWQLNGEELILNMELVETRPAGD | |
| | GTFQKWASVVVPLGKEQYYTCHVYHQGLPEPLTL | |
| | RWE | |
| Human-MHC-I domain- | GSHSMRYFFTSVSRPGRGEPRFIAVGYVDDTQFV | 68 |
| WT | RFDSDAASQRMEPRAPWIEQEGPEYWDGETRKVK | |
| HLA-A2 | AHSQTHRVDLGTLRGCYNQSEAGSHTVQRMYGCD | |
| | VGSDWRFLRGYHQYAYDGKDYIALKEDLRSWTAA | |
| | DMAAQTTKHKWEAAHVAEQLRAYLEGTCVEWLRR | |
| | YLENGKETLQRTDAPKTHMTHHAVSDHEATLRCW | |
| | ALSFYPAEITLTWQRDGEDQTQDTELVETRPAGD | |
| | GTFQKWAAVVVPSGQEQRYTCHVQHEGLPKPLTL | |
| | RWEPSSQPTIP | |
| Human-MHC-I domain- | GSHSMRYFFTSVSRPGRGEPRFIAVGYVDDTQFV | 69 |
| A245V | RFDSDAASQRMEPRAPWIEQEGPEYWDGETRKVK | |
| HLA-A2 CD8 null binder | AHSQTHRVDLGTLRGCYNQSEAGSHTVQRMYGCD | |
| | VGSDWRFLRGYHQYAYDGKDYIALKEDLRSWTAA | |
| | DMAAQTTKHKWEAAHVAEQLRAYLEGTCVEWLRR | |
| | YLENGKETLQRTDAPKTHMTHHAVSDHEATLRCW | |
| | ALSFYPAEITLTWQRDGEDQTQDTELVETRPAGD | |
| | GTFQKWVAVVVPSGQEQRYTCHVQHEGLPKPLTL | |
| | RWEPSSQPTIP | |

LINKERS

| | | |
|---|---|---|
| G₄S | GGGGS | 11 |
| (G₄S)₃ | GGGGSGGGGSGGGGS | 57 |
| (G₄S)₄ | GGGGSGGGGSGGGGSGGGGS | 58 |
| GCGAS | GCGAS | 86 |
| GCGAS-(G4S)₂ | GCGASGGGGSGGGGS | 70 |

STALK/HINGE

| | | |
|---|---|---|
| Mouse-CD28 Stalk | IEFMYPPPYLDNERSNGTIIHIKEKHLCHTQSSP | 63 |
| | KL | |
| Human-CD28 Hinge | GAAAIEVMYPPPYLDNEKSNGTIIHVKGKHLCPS | 71 |
| | PLFPGPSKP | |

TRANSMEMBRANE DOMAIN

| | | |
|---|---|---|
| Mouse-CD28 TM | FWALVVVAGVLFCYGLLVTVALCVIWT | 12 |
| Human-CD28 | FWVLVVVGGVLACYSLLVTVAFIIFWV | 13 |
| transmembrane domain | | |

COSTIMULATORY DOMAINS

| | | |
|---|---|---|
| Mouse-CD28 costimulatory | NSRRNRGGQSDYMNMTPRRPGLTRKPYQPYAPAR | 14 |
| domain | DFAAYRP | |
| Human-CD28 | RSKRSRGGHSDYMNMTPRRPGPTRKHYQPYAPPR | 15 |
| costimulatory domain | DEAAYRS | |
| Human-CD28 | RSKRSRLLHSDYMNMTPRRPGPTRKHYQPYAPPR | 35 |
| costimulatory domain | DEAAYRS | |

INTRACELLULAR SIGNALING DOMAINS

| | | |
|---|---|---|
| Mouse CD3zeta ITAM 2 | RAKFSRSAETAANLQDPNQLFNELNLGRREEFDV | 18 |
| Live | LEKKRARDPEMGGKQQRRRNPQEGVYNALOKDKM | |
| | AEAYSEIGTKGERRRGKGHDGLFQGLSTATKDTF | |
| | DALHMQTLAPR | |
| Mouse CD3zeta Fully | RAKFSRSAETAANLQDPNQLYNELNLGRREEYDV | 19 |
| Live | LEKKRARDPEMGGKQQRRRNPQEGVYNALQKDKM | |
| | AEAYSEIGTKGERRRGKGHDGLYQGLSTATKDTY | |
| | DALHMQTLAPR | |
| Human CD3zeta | DIRVKFSRSADAPAYQQGQNQLYNELNLGRREEY | 16 |
| intracellular | DVLDKRRGRDPEMGGKPRRKNPQEGLYNELQKDK | |
| signaling domain | MAEAYSEIGMKGERRRGKGHDGLYQGLSTATKDT | |
| | YDALHMQALPPR | |
| Human CD3zeta | RVKFSRSADAPAYQQGQNQLYNELNLGRREEYDV | 17 |
| intracellular | LDKRRGRDPEMGGKPRRKNPQEGLYNELQKDKMA | |
| signaling domain | EAYSEIGMKGERRRGKGHDGLYQGLSTATKDTYD | |
| | ALHMQALPPR | |
| Human CD3zeta | VKFSRSADAPAYQQGQNQLYNELNLGRREEYDVL | 72 |
| intracellular | DKRRGRDPEMGGKPRRKNPQEGLYNELQKDKMAE | |
| signaling domain | AYSEIGMKGERRRGKGHDGLYQGLSTATKDTYDA | |
| | LHMQALPPRGS | |

Table 2 provides exemplary nucleic acid sequences of the domains which can be used to encode the CARs described herein. In some embodiments, a nucleic acid sequence encoding a CAR provided herein comprises one or more sequences described in Table 2, or a fragment or portion thereof.

TABLE 2
Exemplary Nucleic Acid Sequences of CAR Domains

| | | |
|---|---|---|
| | | SEQ ID |
| Exemplary CAR domains | Amino Acid Sequence | NO: |

Leader

| | | |
|---|---|---|
| Mouse-B2M Leader domain- | atggccagatctgtgaccctggtgttcctggtgctggtg | 36 |
| WT | tctctgacaggactgtacgcc | |
| Human-B2M Leader domain- | ATGTCACGGAGCGTTGCCTTGGCTGTCTTGGCGCTTTTG | 73 |
| WT | TCTCTGTCCGGACTAGAGGCT | |

Cognate Peptide

| | | |
|---|---|---|
| Ovalbumin Immunodominant | agcatcatcaacttcgagaagctc | 37 |
| Epitope-WT | | |
| (“N4” or “OVA₂₅₇₋₂₆₄”) | | |
| Ovalbumin Peptide Mutant #1 | agcatcatccagttcgagaagctc | 39 |
| SIIQFEKL | | |
| Ovalbumin Peptide Mutant #2 | agcatcatcaccttcgagaagctc | 40 |
| SIITFEKL | | |
| Ovalbumin Peptide Mutant #3 | agcatcatcgtgttcgagaagctc | 41 |
| SIIVFEKL | | |
| NY-ESO peptide NY157-165 | AGTCTGCTCATGTGGATCACACAATGT | 74 |
| WT | | |

B2M

| | | |
|---|---|---|
| Mouse-B2M Domain-1 | atccagaaaacccctcagatccaggtgtacagcagacac | 42 |
| | cctcctgagaacggcaagcccaacatcctgaactgctac | |
| | gtgacccagtttcaccctcctcacatcgagatccagatg | |
| | ctggaaaacggcaagaagatccccaaggtcgagatgagc | |
| | gacatgagcttcagcaaggactggtccttctacatcctg | |
| | gctcacaccgagttcacccctaccgagacagacacctac | |
| | gcctgcagagtgaagcacgcctctatggccgagcctaag | |
| | accgtgtactgggacagagatatg | |
| Human-B2M Domain-1 | ATCCAACGCACACCAAAGATTCAAGTCTATAGCCGACAT | 75 |
| | CCCGCCGAAAATGGCAAGAGTAACTTTCTGAACTGTTAC | |
| | GTGTCCGGATTTCATCCGTCTGATATTGAAGTTGACCTG | |
| | CTTAAGAATGGCGAACGGATTGAGAAAGTGGAACATTCT | |
| | GACCTCTCTTTCTCTAAAGACTGGTCCTTTTATCTATTG | |
| | TACTACACCGAGTTTACACCAACGGAAAAGGACGAATAC | |
| | GCTTGTCGAGTCAACCACGTCACACTGTCCCAACCGAAA | |
| | ATTGTGAAGTGGGATCGGGATATG | |

MHC-I/HLA domain

| | | |
|---|---|---|
| Mouse-MHC-I domain-WT | ggacctcacagcctgagatacttcgtgaccgccgtgtct | 43 |
| H-2KB-WT | agacctggccttggcgagcccagatatatggaagtgggc | |
| | tacgtggacgacacagagttcgtcagattcgacagcgac | |
| | gctgagaaccccagatacgagcctagagccagatggatg | |
| | gaacaagagggccccgagtattgggagagagagacacag | |
| | aaggccaagggcaacgagcagagcttcagagtggacctg | |
| | agaacactgctgggctactacaaccagagcaaaggcggc | |
| | agccacaccatccaagtgatcagcggatgtgaagtgggc | |
| | agcgacggcagactgctgagaggataccagcagtacgcc | |
| | tacgacggctgcgactatatcgccctgaacgaggacctg | |
| | aaaacctggacagccgccgatatggccgctctgatcacc | |
| | aagcacaagtgggagcaagctggcgaggccgagagactg | |
| | agagcttacctggaaggcacctgtgtggaatggctgcgg | |
| | agatacctgaagaacggaaacgccacactgctgaggaca | |
| | gacagccctaaggctcacgtgacacaccacagcagaccc | |
| | gaggacaaagtgaccctgagatgctgggctctgggcttc | |
| | taccctgccgacatcacactgacctggcagctgaatggc | |
| | gaggaactgatccaggacatggaactggtggaaaccaga | |
| | cctgctggcgacggcaccttccagaaatgggcttctgtg | |
| | gtggtgcccctgggcaaagagcagtactacacctgtcac | |
| | gtgtaccaccagggcctgcctgaacctctgacactgaga | |
| | tgggaa | |
| Mouse-MHC-I domain- | ggacctcacagcctgagatacttcgtgaccgccgtgtct | 44 |
| Intermediate CD8 binder | agacctggccttggcgagcccagatatatggaagtgggc | |
| H-2KB From SCT2 | tacgtggacgacacagagttcgtcagattcgacagcgac | |
| | gctgagaaccccagatacgagcctagagccagatggatg | |
| | gaacaagagggccccgagtattgggagagagagacacag | |
| | aaggccaagggcaacgagcagagcttcagagtggacctg | |
| | agaacactgctgggctactacaaccagagcaaaggcggc | |
| | agccacaccatccaagtgatcagcggatgtgaagtgggc | |
| | agcgacggcagactgctgagaggataccagcagtacgcc | |
| | tacgacggctgcgactatatcgccctgaacgaggacctg | |
| | aaaacctggacagccgccgatatggccgctctgatcacc | |
| | aagcacaagtgggagcaagctggcgaggccgagagactg | |
| | agagcttacctggaaggcacctgtgtggaatggctgcgg | |
| | agatacctgaagaacggaaacgccacactgctgaggaca | |
| | gacagccctaaggctcacgtgacacaccacagcagaccc | |
| | gaggacaaagtgaccctgagatgctgggctctgggcttc | |
| | taccctgccgacatcacactgacctggcagctgaatggc | |
| | gaggaactgatccagaacatggaactggtggaaaccaga | |
| | cctgccggcgacggcacattccagaaatgggcatctgtg | |
| | gtggtgcccctgggcaaagagcagtactacacctgtcac | |
| | gtgtaccaccagggcctgcctgaacctctgacactgaga | |
| | tgggaa | |
| Mouse-MHC-I domain-Null | ggacctcacagcctgagatacttcgtgaccgccgtgtct | 45 |
| CD8 binder | agacctggccttggcgagcccagatatatggaagtgggc | |
| H-2KB From SCT3 | tacgtggacgacacagagttcgtcagattcgacagcgac | |
| | gctgagaaccccagatacgagcctagagccagatggatg | |
| | gaacaagagggccccgagtattgggagagagagacacag | |
| | aaggccaagggcaacgagcagagcttcagagtggacctg | |
| | agaacactgctgggctactacaaccagagcaaaggcggc | |
| | agccacaccatccaagtgatcagcggatgtgaagtgggc | |
| | agcgacggcagactgctgagaggataccagcagtacgcc | |
| | tacgacggctgcgactatatcgccctgaacgaggacctg | |
| | aaaacctggacagccgccgatatggccgctctgatcacc | |
| | aagcacaagtgggagcaagctggcgaggccgagagactg | |
| | agagcttacctggaaggcacctgtgtggaatggctgcgg | |
| | agatacctgaagaacggaaacgccacactgctgaggaca | |
| | gacagccctaaggctcacgtgacacaccacagcagaccc | |
| | gaggacaaagtgaccctgagatgctgggctctgggcttc | |
| | taccctgccgacatcacactgacctggcagctgaatggc | |
| | gaggaactgatcctgaacatggaactggtggaaaccaga | |
| | cctgccggcgacggcacattccagaaatgggcatctgtg | |
| | gtggtgcccctgggcaaagagcagtactacacctgtcac | |
| | gtgtaccaccagggcctgcctgaacctctgacactgaga | |
| | tgggaa | |
| Human-MHC-I domain-WT | GGGAGCCACTCAATGAGGTACTTCTTTACTAGCGTAAGT | 76 |
| HLA-A2 | AGGCCGGGTAGAGGTGAACCAAGATTTATTGCAGTCGGG | |
| | TACGTTGACGACACACAGTTCGTGAGATTCGATAGTGAC | |
| | GCCGCGTCCCAACGGATGGAACCACGAGCACCCTGGATA | |
| | GAACAAGAAGGCCCAGAATACTGGGACGGTGAAACAAGG | |
| | AAAGTAAAAGCACATAGCCAGACCCACCGCGTTGATCTC | |
| | GGTACATTGCGGGGTTGCTATAATCAATCCGAAGCCGGG | |
| | TCTCACACGGTGCAAAGGATGTACGGATGTGATGTTGGC | |
| | AGTGACTGGAGATTTCTCAGAGGGTATCACCAATACGCC | |
| | TATGATGGCAAAGACTATATTGCGCTTAAGGAAGATCTC | |
| | CGTTCCTGGACTGCTGCAGATATGGCAGCTCAAACAACG | |
| | AAACATAAATGGGAAGCAGCGCATGTAGCCGAGCAGTTG | |
| | CGAGCTTACCTGGAAGGCACATGTGTGGAATGGCTCAGG | |
| | CGATACCTGGAAAATGGAAAAGAGACACTCCAGCGAACT | |
| | GATGCTCCCAAAACTCATATGACTCATCATGCGGTATCC | |
| | GATCATGAAGCTACTCTGCGCTGCTGGGCACTGAGCTTT | |
| | TATCCAGCCGAGATCACTCTGACCTGGCAACGAGATGGG | |
| | GAAGACCAAACTCAGGACACCGAACTCGTGGAAACAAGA | |
| | CCCGCAGGCGATGGAACCTTCCAAAAGTGGGCGGCAGTG | |
| | GTCGTTCCAAGCGGGCAAGAGCAACGATACACATGTCAC | |
| | GTTCAACACGAAGGACTCCCTAAACCGCTGACACTTCGA | |
| | TGGGAGCCCTCAAGTCAGCCTACAATACCG | |
| Human-MHC-I domain- | GGGAGCCACTCAATGAGGTACTTCTTTACTAGCGTAA | 77 |
| A245V | GTAGGCCGGGTAGAGGTGAACCAAGATTTATTGCAGTCG | |
| HLA-A2 CD8 null binder | GGTACGTTGACGACACACAGTTCGTGAGATTCGATAGTG | |
| | ACGCCGCGTCCCAACGGATGGAACCACGAGCACCCTGGA | |
| | TAGAACAAGAAGGCCCAGAATACTGGGACGGTGAAACAA | |
| | GGAAAGTAAAAGCACATAGCCAGACCCACCGCGTTGATC | |
| | TCGGTACATTGCGGGGTTGCTATAATCAATCCGAAGCCG | |
| | GGTCTCACACGGTGCAAAGGATGTACGGATGTGATGTTG | |
| | GCAGTGACTGGAGATTTCTCAGAGGGTATCACCAATACG | |
| | CCTATGATGGCAAAGACTATATTGCGCTTAAGGAAGATC | |
| | TCCGTTCCTGGACTGCTGCAGATATGGCAGCTCAAACAA | |
| | CGAAACATAAATGGGAAGCAGCGCATGTAGCCGAGCAGT | |
| | TGCGAGCTTACCTGGAAGGCACATGTGTGGAATGGCTCA | |
| | GGCGATACCTGGAAAATGGAAAAGAGACACTCCAGCGAA | |
| | CTGATGCTCCCAAAACTCATATGACTCATCATGCGGTAT | |
| | CCGATCATGAAGCTACTCTGCGCTGCTGGGCACTGAGCT | |
| | TTTATCCAGCCGAGATCACTCTGACCTGGCAACGAGATG | |
| | GGGAAGACCAAACTCAGGACACCGAACTCGTGGAAACAA | |
| | GACCCGCAGGCGATGGAACCTTCCAAAAGTGGGTGGCAG | |
| | TGGTCGTTCCAAGCGGGCAAGAGCAACGATACACATGTC | |
| | ACGTTCAACACGAAGGACTCCCTAAACCGCTGACACTTC | |
| | GATGGGAGCCCTCAAGTCAGCCTACAATACCG | |

LINKERS

| | | |
|---|---|---|
| (G₄S)₃ | gcggaggcggatctggtggcggaggaagtggcggcggag | 60 |
| | gatct | |
| (G₄S)₄ | gaggcggtggaagcggaggtggtggatctggcggtggcg | 61 |
| | gtagcggaggcggaggttct | |
| (G₄S)₄ | GGTGGTGGCGGAAGCGGTGGTGGTGGATCAGGTGGCGGC | 78 |
| | GGCAGTGGTGGTGGTGGATCT | |
| GCGAS-(G4S)₂ | GGATGTGGTGCTTCTGGCGGTGGCGGAAGTGGTGGTGGC | 79 |
| | GGGAGC | |

STALK/HINGE

| | | |
|---|---|---|
| Mouse-CD28 Stalk | atcgagttcatgtaccctccaccttacctggacaacgag | 64 |
| | agaagcaacggcaccatcatccacatcaaagagaagcac | |
| | ctctgtcacacccagagcagccccaagctg | |
| Human-CD28 Hinge | GGCGCCGCTGCTATTGAAGTGATGTACCCTCCTCCTTAC | 80 |
| | CTGGACAACGAGAAGTCCAACGGCACCATCATCCACGTG | |
| | AAGGGCAAGCACCTGTGTCCTTCTCCACTGTTCCCCGGA | |
| | CCTAGCAAGCCT | |

TRANSMEMBRANE DOMAINS

| | | |
|---|---|---|
| Mouse-CD28 TM | ttttgggctcttgtggtggtggctggcgtgctgttttgt | 47 |
| | tacggcctgctcgtgacagtggccctgtgtgtgatctgg | |
| | acc | |
| Human-CD28 transmembrane | ttctgggtgctcgttgttgttggcggcgtgctggcctgt | 46 |
| domain | tacagcctgctggttaccgtggccttcatcatcttttgg | |
| | gtg | |
| Human-CD28 transmembrane | TTCTGGGTGCTCGTTGTTGTTGGCGGCGTGCTGGCCTGT | 81 |
| domain | TATTCCCTGCTGGTTACCGTGGCCTTCATCATCTTTTGG | |
| | GTC | |

COSTIMULATORY DOMAINS

| | | |
|---|---|---|
| Mouse-CD28 costimulatory | aacagcagaagaaacagaggcggccagagcgactacatg | 50 |
| domain | aacatgacccctagaaggcctggcctgaccagaaagcct | |
| | taccagccttacgctcccgccagagatttcgccgcttat | |
| | agacca | |
| Human-CD28 costimulatory | cgaagcaagcggagccggggaggacacagcgactacatg | 48 |
| domain | aacatgacccctcggaggccaggccccaccagaaagcac | |
| | taccagccctacgcccctccccgggactttgccgcctat | |
| | cggagc | |
| Human-CD28 costimulatory | cgaagcaagcggagccggctgctgcacagcgactacatg | 49 |
| domain | aacatgacccctcggaggccaggccccaccagaaagcac | |
| | taccagccctacgcccctccccgggactttgccgcctat | |
| | cggagc | |
| Human-CD28 costimulatory | CGAAGCAAGCGGAGCCGGCTGCTGCACAGCGACTACATG | 82 |
| domain | AACATGACCCCTAGACGGCCCGGACCAACCAGAAAGCAC | |
| | TACCAGCCTTACGCTCCTCCTAGAGACTTCGCCGCCTAC | |
| | CGG | |

INTRACELLULAR SIGNALING

| | | |
|---|---|---|
| Mouse-CD3zeta ITAM 2 live | agagccaagttctctagatctgccgagacagccgccaac | 53 |
| | ctgcaggaccctaaccagctgttcaacgagctgaacctg | |
| | gggagaagagaagagttcgacgtgctggaaaagaagaga | |
| | gccagagatcccgagatgggcggcaagcagcagagaaga | |
| | agaaaccctcaagagggcgtgtacaacgctctgcagaaa | |
| | gacaagatggccgaggcctacagcgagatcggaactaag | |
| | ggcgaacgcagaagaggcaagggccacgatggactgttt | |
| | cagggcctgagcacagccaccaaggacacattcgatgcc | |
| | ctgcacatgcagacactggcccctaga | |
| Mouse-CD3zeta fully live | agagccaagttctccagatctgctgagacagccgccaac | 54 |
| | ctgcaggaccctaaccagctgtacaacgagctgaacctg | |
| | gggagaagagaagagtacgacgtgctggaaaagaagagg | |
| | gccagagatcctgagatgggcggcaagcagcagagaaga | |
| | agaaaccctcaagaaggcgtgtacaacgccctgcagaaa | |
| | gacaagatggccgaggcctacagcgagatcggaactaag | |
| | ggcgaacgcagaagaggcaagggccacgatggactgtac | |
| | cagggcctgtctacagccaccaaggacacatacgatgcc | |
| | ctgcacatgcagacactggcccctaga | |
| human-CD3zeta intracellular | gatatcagggtgaagttcagcaggagcgccgacgccccc | 51 |
| signaling domain | gcttatcaacagggccagaaccagctgtacaacgagctg | |
| | aacctcggcagaagagaggagtatgacgtgctggacaag | |
| | aggaggggcagggaccctgagatgggcggcaagcctaga | |
| | agaaagaacccccaggaaggcctctacaacgaactgcag | |
| | aaggacaagatggccgaggcctacagcgagatcggcatg | |
| | aaaggcgagagaaggaggggaaagggacatgacggcctg | |
| | taccagggactctccacagccaccaaggacacctacgat | |
| | gccctgcacatgcaggctctgccccctaga | |
| human-CD3zeta intracellular | agggtgaagttcagcaggagcgccgacgcccccgcttat | 52 |
| signaling domain | caacagggccagaaccagctgtacaacgagctgaacctc | |
| | ggcagaagagaggagtatgacgtgctggacaagaggagg | |
| | ggcagggaccctgagatgggcggcaagcctagaagaaag | |
| | aacccccaggaaggcctctacaacgaactgcagaaggac | |
| | aagatggccgaggcctacagcgagatcggcatgaaaggc | |
| | gagagaaggaggggaaagggacatgacggcctgtaccag | |
| | ggactctccacagccaccaaggacacctacgatgccctg | |
| | cacatgcaggctctgccccctaga | |
| human-CD3zeta intracellular | GTGAAGTTCAGCAGATCCGCCGACGCTCCTGCCTATCAG | 83 |
| signaling domain | CAGGGACAGAACCAGCTGTACAACGAGCTGAACCTGGGG | |
| | AGAAGAGAAGAGTACGACGTGCTGGACAAGCGGAGAGGC | |
| | AGAGATCCTGAGATGGGCGGCAAGCCCAGACGGAAGAAT | |
| | CCTCAAGAGGGCCTGTATAATGAGCTGCAGAAAGACAAG | |
| | ATGGCCGAGGCCTACAGCGAGATCGGAATGAAGGGCGAG | |
| | CGCAGAAGAGGCAAGGGACACGATGGACTGTACCAGGGC | |
| | CTGAGCACCGCCACCAAGGATACCTATGATGCCCTGCAC | |
| | ATGCAGGCCCTGCCTCCAAGAGGATCC | |

G. Exemplary Bait CAR Constructs that Specifically Bind CD8+ T-Cells

Disclosed herein are CARs that specifically bind to T-cells. In some embodiments, the CARs specifically bind to CD8+ T-cells. In some embodiments, the CAR comprises an ectodomain comprising a cognate peptide that specifically binds a T-cell receptor. In some embodiments the CAR comprises an ectodomain comprising a cognate peptide that is recognized by a T-cell receptor of a CD8+ T-cell (i.e. ectodomain comprising a cognate peptide that specifically binds a T-cell receptor of a CD8+ T-cell).

In some embodiments, the CAR comprises an ectodomain comprising (a) an ectodomain comprising i. a beta-2 microglobulin leader peptide (B2ML), ii. a cognate peptide that is recognized by a CD8+ T-cell Receptor (P), iii. at least one linker domain (L), iv. a beta-2 microglobulin peptide (B2M), v. a MHC class I (MHCI), a HLA-A, a HLA-B or a HLA-C domain; and vi. a stalk/hinge domain (b) a transmembrane domain: (c) at least one costimulatory domain; and (d) an intracellular signaling domain.

In some embodiments, the CAR comprises an ectodomain comprising (a) an ectodomain comprising i. a beta-2 microglobulin leader peptide (B2ML), ii. a cognate peptide that is recognized by a CD8+ T-cell Receptor (P), iii. at least one linker domain (L), iv. a beta-2 microglobulin peptide (B2M), v. a MHC class I (MHCI) domain; and vi. a stalk/hinge domain (b) a transmembrane domain comprising or consisting of a CD28 transmembrane domain: (c) at least one costimulatory domain comprising or consisting of a CD28 costimulatory domain; and (d) an intracellular signaling domain comprising or consisting of a CD3zeta intracellular signaling domain.

In some embodiments, the CAR comprises (a) an ectodomain comprising the following in the N-terminal to C-terminal direction: B2ML-P-(L) x-B2M-(L)-(MHCI), (b) a transmembrane domain comprising or consisting of a CD28 transmembrane domain: (c) at least one costimulatory domain comprising or consisting of a CD28 costimulatory domain; and (d) an intracellular signaling domain comprising or consisting of a CD3zeta intracellular signaling domain, wherein x is any integer between 0-5; and wherein y is any integer between 0-5. In some embodiments, the CAR comprises (a) an ectodomain comprising the following in the N-terminal to C-terminal direction: B2ML-P-(L) 4-B2M-(L) 3-(MHCI), (b) a transmembrane domain comprising or consisting of a CD28 transmembrane domain: (c) at least one costimulatory domain comprising or consisting of a CD28 costimulatory domain; and (d) an intracellular signaling domain comprising or consisting of a CD3zeta intracellular signaling domain. In some embodiments L comprises the amino acid sequence of SEQ ID NO: 11.

In some embodiments, the CAR comprises (a) an ectodomain comprising the following in the N-terminal to C-terminal direction: N-term-B2ML-P-(Linker 1)ₓ-(Linker 2) z-B2M-(Linker 2) y-(HLA-A/HLA-B/HLA-C), (b) a transmembrane domain comprising or consisting of a CD28 transmembrane domain: (c) at least one costimulatory domain comprising or consisting of a CD28 costimulatory domain; and (d) an intracellular signaling domain comprising or consisting of a CD3zeta intracellular signaling domain, wherein x is any integer between 0-5: wherein y is any integer between 0-5; and wherein z is any integer between 0-5. In some embodiments, the CAR comprises (a) an ectodomain comprising the following in the N-terminal to C-terminal direction: N-term-B2ML-P-(Linker 1) 1-(Linker 2) 2-B2M-(Linker 2)₃-(HLA-A/HLA-B/HLA-C). In some embodiments, Linker 1 comprises the amino acid sequence of SEQ ID NO: 86. In some embodiments, Linker 2 comprises the amino acid sequence of SEQ ID NO: 11.

Also disclosed herein are nucleic acid sequences encoding said CARs. In some embodiments, a T cell or population of T cells described herein is genetically modified to express at least one of the exemplary Bait-CAR constructs described herein.

An exemplary “OVA-MHCI Bait-SIINFEKL” CAR amino acid sequence is shown below (B2M leader peptide, Cognate peptide, linker, B2M domain, linker, MHCI domain, linker, CD28 stalk. CD28 transmembrane, CD28 costimulatory domain, linker CD3z signaling domain).

(SEQ ID NO: 20)
MARSVTLVFLVLVSLTGLYASIINFEKLGGGSGGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVRFDSDAENPRYEPRARWMEQEGPEYWERETQKAKGNEQSFRVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELIQDMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTFDALHMQTLAPR

In some embodiments, the “OVA-MHCI Bait-SIINFEKL” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 20.

An exemplary “OVA-MHCI Bait-SIINFEKL” CAR polynucleotide sequence is shown below (B2M leader peptide, Cognate peptide, linker, B2M domain, linker, MHCI domain, linker CD28 stalk, CD28 transmembrane, CD28 costimulatory domain, linker CD3z signaling domain).

(SEQ ID NO: 21)
ATGGCCAGATCTGTGACCCTGGTGTTCCTGGTGCTGGTGTCTCTGACAGGACTGTACGCCAG
CATCATCAACTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCAGGACATGGAACTGGT
GGAAACCAGACCTGCTGGCGACGGCACCTTCCAGAAATGGGCTTCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-SIINFEKL” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 21.

An exemplary “OVA-MHCI Bait-SIIQFEKL” CAR amino acid sequence is shown below (B2M leader peptide, Cognate peptide, linker, B2M domain, linker, MHCI domain, linker, CD28 stalk. CD28 transmembrane, CD28 costimulatory domain, linker CD3z signaling domain).

(SEQ ID NO: 22)
MARSVTLVFLVLVSLTGLYASIIQFEKLGGGGSGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVRfDSDAENPRYEPRARWMEQEGPEYWERETQKAKGNEQSFRVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELIQDMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTFDALHMQTLAPR

In some embodiments, the “OVA-MHCI Bait-SIIQFEKL” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 22.

An exemplary “OVA-MHCI Bait-SIIQFEKL” CAR polynucleotide sequence is shown below (B2M leader peptide, Cognate peptide, linker, B2M domain, linker, MHCI domain, linker, CD28 transmembrane, CD28 costimulatory domain, linker CD3z signaling domain).

(SEQ ID NO: 23)
ATGGCCAGATCTGTGACCCTGGTGTTCCTGGTGCTGGTGTCTCTGACAGGACTGTACGCCAG
CATCATCCAGTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCAGGACATGGAACTGGT
GGAAACCAGACCTGCTGGCGACGGCACCTTCCAGAAATGGGCTTCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-SIIQFEKL” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 23.

An exemplary “OVA-MHCI Bait-SIITFEKL” CAR amino acid sequence is shown below (B2M leader peptide, Cognate peptide, linker, B2M domain, linker, MHCI domain, linker CD28 stalk, CD28 transmembrane, CD28 costimulatory domain, linker CD3z signaling domain).

(SEQ ID NO: 24)
MARSVTLVFLVLVSLTGLYASIITFEKLGGGGSGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVRFDSDAENPRYEPRARWMEQEGPEYWERETQKAKGNEQSFRVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELIQDMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTEDALHMQTLAPR

In some embodiments, the “OVA-MHCI Bait-SIITFEKL” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 24.

An exemplary “OVA-MHCI Bait-SIITFEKL” CAR polynucleotide sequence is shown below (B2M leader peptide, Cognate peptide, linker, B2M domain, linker, MHCI domain, linker, CD28 transmembrane, CD28 costimulatory domain, linker CD3z signaling domain).

(SEQ ID NO: 25)
ATGGCCAGATCTGTGACCCTGGTGTTCCTGGTGCTGGTGTCTCTGACAGGACTGTACGCCAG
CATCATCACCTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCAGGACATGGAACTGGT
GGAAACCAGACCTGCTGGCGACGGCACCTTCCAGAAATGGGCTTCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-SIITFEKL” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 25.

An exemplary “OVA-MHCI Bait-SIIVFEKL” CAR amino acid sequence is shown below (B2M leader peptide, Cognate peptide, linker, B2M domain, linker, MHCI domain, CD 28 stalk. CD28 transmembrane, CD28 costimulatory domain, CD3z signaling domain).

(SEQ ID NO: 26)
MARSVTLVFLVLVSLTGLYASIIVFEKLGGGGSGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVRFDSDAENPRYEPRARWMEQEGPEYWERETQKAKGNEQSFRVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELIQDMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTEDALHMQTLAPR

In some embodiments, the “OVA-MHCI Bait-SIIVFEKL” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 26.

An exemplary “OVA-MHCI Bait-2” CAR polynucleotide sequence is shown below (B2M leader peptide. Cognate peptide, linker, B2M domain, linker, MHCI domain, linker, CD28 transmembrane, CD28 costimulatory domain, linker CD3z signaling domain).

(SEQ ID NO: 27)
ATGGCCAGATCTGTGACCCTGGTGTTCCTGGTGCTGGTGTCTCTGACAGGACTGTACGCCAG
CATCATCGTGTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCAGGACATGGAACTGGT
GGAAACCAGACCTGCTGGCGACGGCACCTTCCAGAAATGGGCTTCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-SIIVFEKL” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 27.

An exemplary “OVA-MHCI Bait-CD8 Intermediate Binder” CAR amino acid sequence is shown below (B2M leader peptide, Cognate peptide, linker, B2M domain, linker, MHCI domain, linker, CD28 stalk, CD28 transmembrane, CD28 costimulatory domain, linker, CD3z signaling domain).

(SEQ ID NO: 28)
MARSVTLVFLVLVSLTGLYASIINFEKLGGGGSGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVRFDSDAENPRYEPRARWMEQEGPEYWERETQKAKGNEQSERVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELILNMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTEDALHMQTLAPR

In some embodiments, the “OVA-MHCI Bait-CD8 Intermediate Binder” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 28.

An exemplary “OVA-MHCI Bait-CD8 Intermediate Binder” CAR polynucleotide sequence is shown below (B2M leader peptide, Cognate peptide, linker, B2M domain linker, MHCI domain, linker, CD28 transmembrane, CD28 costimulatory domain, linker, CD3z signaling domain).

(SEQ ID NO: 29)
ATGGCCAGATCTGTGACCCTGGTGTTCCTGGTGCTGGTGTCTCTGACAGGACTGTACGCCAG
CATCATCAACTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCAGAACATGGAACTGGT
GGAAACCAGACCTGCCGGCGACGGCACATTCCAGAAATGGGCATCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-CD8 Intermediate Binder” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 29.

An exemplary “OVA-MHCI Bait-CD8 Null Binder” CAR amino acid sequence is shown below (B2M leader peptide, Cognate peptide, linker, B2M domain, linker, MHCI domain, linker, CD28 stalk, CD28 transmembrane, CD28 costimulatory domain, linker,

CD3z signaling domain).

(SEQ ID NO: 30)
MARSVTLVFLVLVSLTGLYASIINFEKLGGGGSGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVREDSDAENPRYEPRARWMEQEGPEYWERETQKAKGNEQSFRVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELIQNMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTEDALHMQTLAPR

In some embodiments, the “OVA-MHCI Bait-CD8 Null Binder” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 30.

An exemplary “OVA-MHCI Bait-2” CAR polynucleotide sequence is shown below (B2M leader peptide, Cognate peptide, linker, B2M domain, linker, MHCI domain, linker, CD28 transmembrane CD28 costimulatory domain, linker (D37 signaling domain)

(SEQ ID NO: 31)
ATGGCCAGATCTGTGACCCTGGTGTTCCTGGTGCTGGTGTCTCTGACAGGACTGTACGCCAG
CATCATCAACTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCTGAACATGGAACTGGT
GGAAACCAGACCTGCCGGCGACGGCACATTCCAGAAATGGGCATCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-CD8 Null Binder” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 31.

An exemplary “OVA-MHCI Bait-CD3Zeta Fully Live” CAR amino acid sequence is shown below (B2M leader peptide, Cognate peptide, linker, B2M domain, linker, MHCI domain, linker, CD28 stalk, CD28 transmembrane, CD28 costimulatory domain, linker, CD3z signaling domain).

(SEQ ID NO: 32)
MARSVTLVFLVLVSLTGLYASIINFEKLGGGGSGGGGSGGGGSIQKTPQIQVYSRHPPENGK
PNILNCYVTQFHPPHIEIQMLENGKKIPKVEMSDMSFSKDWSFYILAHTEFTPTETDTYACR
VKHASMAEPKTVYWDRDMGGGGSGGGGSGGGGSGGGGSGPHSLRYFVTAVSRPGLGEPRYME
VGYVDDTEFVRFDSDAENPRYEPRARWMEQEGPEYWERETQKAKGNEQSERVDLRTLLGYYN
QSKGGSHTIQVISGCEVGSDGRLLRGYQQYAYDGCDYIALNEDLKTWTAADMAALITKHKWE
QAGEAERLRAYLEGTCVEWLRRYLKNGNATLLRTDSPKAHVTHHSRPEDKVTLRCWALGFYP
ADITLTWQLNGEELIQDMELVETRPAGDGTFQKWASVVVPLGKEQYYTCHVYHQGLPEPLTL
RWEGSIEFMYPPPYLDNERSNGTIIHIKEKHLCHTQSSPKLFWALVVVAGVLFCYGLLVTVA
LCVIWTNSRRNRGGQSDYMNMTPRRPGLTRKPYQPYAPARDFAAYRPPRRAKFSRSAETAAN
LQDPNQLFNELNLGRREEFDVLEKKRARDPEMGGKQQRRRNPQEGVYNALQKDKMAEAYSEI
GTKGERRRGKGHDGLFQGLSTATKDTEDALHMQTLAPR

In some embodiments, the “OVA-MHCI Bait-CD3Zeta Fully Live” CAR provided herein may comprise or consist of an amino acid sequence having at least 90%, 91%, 92%, 93%, 94%, 95%, 96%, 97%, 98%, 99% or 100% identity with the amino acid sequence of SEQ ID NO: 32.

An exemplary “OVA-MHCI Bait-CD3Zeta Fully Live” CAR polynucleotide sequence is shown below (B2M leader peptide, Cognate peptide, linker, B2M domain linker, MHCI domain, linker, CD28 transmembrane, CD28 costimulatory domain, linker CD3z signaling domain).

(SEQ ID NO: 33)
ATGGCCAGATCTGTGACCCTGGTGTTCCTGGTGCTGGTGTCTCTGACAGGACTGTACGCCAG
CATCATCAACTTCGAGAAGCTCGGCGGAGGCGGATCTGGTGGCGGAGGAAGTGGCGGCGGAG
GATCTATCCAGAAAACCCCTCAGATCCAGGTGTACAGCAGACACCCTCCTGAGAACGGCAAG
CCCAACATCCTGAACTGCTACGTGACCCAGTTTCACCCTCCTCACATCGAGATCCAGATGCT
GGAAAACGGCAAGAAGATCCCCAAGGTCGAGATGAGCGACATGAGCTTCAGCAAGGACTGGT
CCTTCTACATCCTGGCTCACACCGAGTTCACCCCTACCGAGACAGACACCTACGCCTGCAGA
GTGAAGCACGCCTCTATGGCCGAGCCTAAGACCGTGTACTGGGACAGAGATATGGGAGGCGG
TGGAAGCGGAGGTGGTGGATCTGGCGGTGGCGGTAGCGGAGGCGGAGGTTCTGGACCTCACA
GCCTGAGATACTTCGTGACCGCCGTGTCTAGACCTGGCCTTGGCGAGCCCAGATATATGGAA
GTGGGCTACGTGGACGACACAGAGTTCGTCAGATTCGACAGCGACGCTGAGAACCCCAGATA
CGAGCCTAGAGCCAGATGGATGGAACAAGAGGGCCCCGAGTATTGGGAGAGAGAGACACAGA
AGGCCAAGGGCAACGAGCAGAGCTTCAGAGTGGACCTGAGAACACTGCTGGGCTACTACAAC
CAGAGCAAAGGCGGCAGCCACACCATCCAAGTGATCAGCGGATGTGAAGTGGGCAGCGACGG
CAGACTGCTGAGAGGATACCAGCAGTACGCCTACGACGGCTGCGACTATATCGCCCTGAACG
AGGACCTGAAAACCTGGACAGCCGCCGATATGGCCGCTCTGATCACCAAGCACAAGTGGGAG
CAAGCTGGCGAGGCCGAGAGACTGAGAGCTTACCTGGAAGGCACCTGTGTGGAATGGCTGCG
GAGATACCTGAAGAACGGAAACGCCACACTGCTGAGGACAGACAGCCCTAAGGCTCACGTGA
CACACCACAGCAGACCCGAGGACAAAGTGACCCTGAGATGCTGGGCTCTGGGCTTCTACCCT
GCCGACATCACACTGACCTGGCAGCTGAATGGCGAGGAACTGATCCAGGACATGGAACTGGT
GGAAACCAGACCTGCTGGCGACGGCACCTTCCAGAAATGGGCTTCTGTGGTGGTGCCCCTGG
GCAAAGAGCAGTACTACACCTGTCACGTGTACCACCAGGGCCTGCCTGAACCTCTGACACTG
AGATGGGAAGGATCCATCGAGTTCATGTACCCTCCACCTTACCTGGACAACGAGAGAAGCAA
CGGCACCATCATCCACATCAAAGAGAAGCACCTCTGTCACACCCAGAGCAGCCCCAAGCTGT
TTTGGGCTCTTGTGGTGGTGGCTGGCGTGCTGTTTTGTTACGGCCTGCTCGTGACAGTGGCC
CTGTGTGTGATCTGGACCAACAGCAGAAGAAACAGAGGCGGCCAGAGCGACTACATGAACAT
GACCCCTAGAAGGCCTGGCCTGACCAGAAAGCCTTACCAGCCTTACGCTCCCGCCAGAGATT
TCGCCGCTTATAGACCACCGCGGAGAGCCAAGTTCTCTAGATCTGCCGAGACAGCCGCCAAC
CTGCAGGACCCTAACCAGCTGTTCAACGAGCTGAACCTGGGGAGAAGAGAAGAGTTCGACGT
GCTGGAAAAGAAGAGAGCCAGAGATCCCGAGATGGGCGGCAAGCAGCAGAGAAGAAGAAACC
CTCAAGAGGGCGTGTACAACGCTCTGCAGAAAGACAAGATGGCCGAGGCCTACAGCGAGATC
GGAACTAAGGGCGAACGCAGAAGAGGCAAGGGCCACGATGGACTGTTTCAGGGCCTGAGCAC
AGCCACCAAGGACACATTCGATGCCCTGCACATGCAGACACTGGCCCCTAGA

In some embodiments, the “OVA-MHCI Bait-CD3Zeta Fully Live” CAR provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 33.

An exemplary human “HLA-A2-NYESO bait CAR” or “NYESO-SCT_28z bait CAR” amino acid sequence is shown below (B2M leader peptide, Cognate peptide, linker, B2M domain, linker, HLA-A2 domain, linker, CD28 hinge, CD28 transmembrane, CD28 costimulatory domain, linker, CD3z signaling domain).

(SEQ ID NO: 84)
MSRSVALAVLALLSLSGLEASLLMWITQCGCGASGGGGSGGGGSIQRTPKIQVYSRHPAENG
KSNFLNCYVSGFHPSDIEVDLLKNGERIEKVEHSDLSFSKDWSFYLLYYTEFTPTEKDEYAC
RVNHVTLSQPKIVKWDRDMGGGGSGGGGSGGGGSGGGGSGSHSMRYFFTSVSRPGRGEPRFI
AVGYVDDTQFVRFDSDAASQRMEPRAPWIEQEGPEYWDGETRKVKAHSQTHRVDLGTLRGCY
NQSEAGSHTVQRMYGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSDHEATLRCWALSFY
PAEITLTWQRDGEDQTQDTELVETRPAGDGTFQKWAAVVVPSGQEQRYTCHVQHEGLPKPLT
LRWEPSSQPTIPTSGAAAIEVMYPPPYLDNEKSNGTIIHVKGKHLCPSPLFPGPSKPFWVLV
VVGGVLACYSLLVTVAFIIFWVRSKRSRLLHSDYMNMTPRRPGPTRKHYQPYAPPRDFAAYR
SRVKESRSADAPAYQQGQNQLYNELNLGRREEYDVLDKRRGRDPEMGGKPRRKNPQEGLYNE
LQKDKMAEAYSEIGMKGERRRGKGHDGLYQGLSTATKDTYDALHMQALPPRGS.

In some embodiments, the “HLA-A2-NYESO bait CAR” or “NYESO-SCT_28z bait CAR” provided herein may comprise or consist of an amino acid sequence having at least 90%, 91%, 92%, 93%, 94%, 95%, 96%, 97%, 98%, 99% or 100% identity with the amino acid sequence of SEQ ID NO: 84.

An exemplary “HLA-A2-NYESO” CAR polynucleotide sequence is shown below (B2M leader peptide, Cognate peptide, linker, B2M domain, linker, HLA-A2 domain, linker, CD28 hinge, CD28 transmembrane, CD28 costimulatory domain, linker, CD3z signaling domain).

(SEQ ID NO: 85)
ATGTCACGGAGCGTTGCCTTGGCTGTCTTGGCGCTTTTGTCTCTGTCCGGACTAGAGGCTAG
TCTGCTCATGTGGATCACACAATGTGGATGTGGTGCTTCTGGCGGTGGCGGAAGTGGTGGTG
GCGGGAGCATCCAACGCACACCAAAGATTCAAGTCTATAGCCGACATCCCGCCGAAAATGGC
AAGAGTAACTTTCTGAACTGTTACGTGTCCGGATTTCATCCGTCTGATATTGAAGTTGACCT
GCTTAAGAATGGCGAACGGATTGAGAAAGTGGAACATTCTGACCTCTCTTTCTCTAAAGACT
GGTCCTTTTATCTATTGTACTACACCGAGTTTACACCAACGGAAAAGGACGAATACGCTTGT
CGAGTCAACCACGTCACACTGTCCCAACCGAAAATTGTGAAGTGGGATCGGGATATGGGTGG
TGGCGGAAGCGGTGGTGGTGGATCAGGTGGCGGCGGCAGTGGTGGTGGTGGATCTGGGAGCC
ACTCAATGAGGTACTTCTTTACTAGCGTAAGTAGGCCGGGTAGAGGTGAACCAAGATTTATT
GCAGTCGGGTACGTTGACGACACACAGTTCGTGAGATTCGATAGTGACGCCGCGTCCCAACG
GATGGAACCACGAGCACCCTGGATAGAACAAGAAGGCCCAGAATACTGGGACGGTGAAACAA
GGAAAGTAAAAGCACATAGCCAGACCCACCGCGTTGATCTCGGTACATTGCGGGGTTGCTAT
AATCAATCCGAAGCCGGGTCTCACACGGTGCAAAGGATGTACGGATGTGATGTTGGCAGTGA
CTGGAGATTTCTCAGAGGGTATCACCAATACGCCTATGATGGCAAAGACTATATTGCGCTTA
AGGAAGATCTCCGTTCCTGGACTGCTGCAGATATGGCAGCTCAAACAACGAAACATAAATGG
GAAGCAGCGCATGTAGCCGAGCAGTTGCGAGCTTACCTGGAAGGCACATGTGTGGAATGGCT
CAGGCGATACCTGGAAAATGGAAAAGAGACACTCCAGCGAACTGATGCTCCCAAAACTCATA
TGACTCATCATGCGGTATCCGATCATGAAGCTACTCTGCGCTGCTGGGCACTGAGCTTTTAT
CCAGCCGAGATCACTCTGACCTGGCAACGAGATGGGGAAGACCAAACTCAGGACACCGAACT
CGTGGAAACAAGACCCGCAGGCGATGGAACCTTCCAAAAGTGGGCGGCAGTGGTCGTTCCAA
GCGGGCAAGAGCAACGATACACATGTCACGTTCAACACGAAGGACTCCCTAAACCGCTGACA
CTTCGATGGGAGCCCTCAAGTCAGCCTACAATACCGACTAGTGGCGCCGCTGCTATTGAAGT
GATGTACCCTCCTCCTTACCTGGACAACGAGAAGTCCAACGGCACCATCATCCACGTGAAGG
GCAAGCACCTGTGTCCTTCTCCACTGTTCCCCGGACCTAGCAAGCCTTTCTGGGTGCTCGTT
GTTGTTGGCGGCGTGCTGGCCTGTTATTCCCTGCTGGTTACCGTGGCCTTCATCATCTTTTG
GGTCCGAAGCAAGCGGAGCCGGCTGCTGCACAGCGACTACATGAACATGACCCCTAGACGGC
CCGGACCAACCAGAAAGCACTACCAGCCTTACGCTCCTCCTAGAGACTTCGCCGCCTACCGG
TCTAGAGTGAAGTTCAGCAGATCCGCCGACGCTCCTGCCTATCAGCAGGGACAGAACCAGCT
GTACAACGAGCTGAACCTGGGGAGAAGAGAAGAGTACGACGTGCTGGACAAGCGGAGAGGCA
GAGATCCTGAGATGGGCGGCAAGCCCAGACGGAAGAATCCTCAAGAGGGCCTGTATAATGAG
CTGCAGAAAGACAAGATGGCCGAGGCCTACAGCGAGATCGGAATGAAGGGCGAGCGCAGAAG
AGGCAAGGGACACGATGGACTGTACCAGGGCCTGAGCACCGCCACCAAGGATACCTATGATG
CCCTGCACATGCAGGCCCTGCCTCCAAGAGGATCCTAA.

In some embodiments, the “HLA-A2-NYESO bait CAR” or “NYESO-SCT_28z bait CAR” provided herein is encoded by a polynucleotide sequence comprising or consisting of an nucleic acid sequence having at least 90%, 91%, 92%, 93%, 94%, 95%, 96%, 97%, 98%, 99% or 100% identity with the nucleic acid sequence of SEQ ID NO: 85.

### 4. CAR Expression Levels

The present disclosure provides a population of engineered T cells, wherein a plurality of the engineered T cells of the population comprise any chimeric stimulatory receptor (CAR) disclosed herein. In some embodiments, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% of the population comprise the CAR. In some embodiments, each CAR polypeptide is expressed at a copy number of at least 1, 2, 3, 4, 5, 6, 7, 8, 9, 10, 20, 30, 40, 50, 60, 70, 80, 90 or 100 copies per cell. In some embodiments, the nucleic acid encoding the CAR is integrated into the genome at a copy number of at least 1, 2, 3, 4, 5, 6, 7, 8, 9, 10, 20 or 30 copies per cell.

### 5. Target Antigens

Provided herein are immune cells (e.g., T cells) expressing a CAR that targets CD8+T cells. In some embodiments, the immune cells (e.g. T cells) expressing a CAR targets pathogenic T cells. Among the antigens that may be targeted by the genetically engineered antigen receptors are those expressed in the context of a disease, condition, or cell type to be targeted via the adoptive cell therapy. Among the diseases and conditions are aberrant or misregulated immune responses such as cancers, autoimmune disorders, diseases of immunity, and conditions characterized by chronic inflammation. Aberrant or pathological immune activation underlies diseases, such as autoimmune diseases, solid transplant rejection, transplantation graft rejection, graft versus host disease, allergy, asthma, diabetes mellitus and rheumatoid arthritis and T cell leukemia.

In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells. In other embodiments, the antigen is a hybrid or neoantigen, or alloantigen.

Any suitable antigen may find use in the present method. Exemplary target antigens include, but are not limited to, antigens expressed on the surface of CD8+ T cells, and T cell receptors that specifically bind cognate antigens expressed in the ectodomain of the CARs described herein. In some embodiments, the target antigen is an OT-I TCR. In some embodiments, the target antigen is an NYESO-1 TCR.

6. T cell activity

In some embodiments, a population of genetically engineered T cells as disclosed herein exhibits T cell functions (e.g., effector functions). In some embodiments, the population is cytotoxic to CD8+ T cells (e.g. pathogenic T cell) that recognizes (i.e. specifically binds to) the cognate peptide of the ectodomain of the CAR. Effector function of a genetically engineered T cell, for example, may be cytolytic activity or helper activity including the secretion of cytokines. In some embodiments, the population exhibits one or more T cell effector functions at a level that is least 3-4-fold higher than the functions exhibited by a population of T cells not expressing the CAR.

### III. Methods

Chimeric antigen receptors may be readily inserted into and expressed by immune cells, (e.g., T cells). In certain embodiments, cells (e.g., immune cells such as T cells) are obtained from a donor subject. In some embodiments, the donor subject is human patient afflicted with autoimmune disease or transplant rejection. In other embodiments, the donor subject is a human patient not afflicted with autoimmune disease or transplant rejection. In some embodiments, an engineered cell is autologous to a subject. In some embodiments, an engineered cell is allogeneic to a subject.

The cell of the present disclosure may be obtained through any source known in the art. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. Additional methods of isolating T cells for a T cell therapy are disclosed in U.S. Patent Publication No. 2013/0287748, which is herein incorporated by references in its entirety.

In some embodiments. PBMCs are used directly for genetic modification with the immune cells (such as CARs) using methods as described herein. In certain embodiments, after isolating the PBMCs. T lymphocytes are further isolated, and both cytotoxic and helper T lymphocytes are sorted into naive, memory, and effector T cell subpopulations either before or after genetic modification and/or expansion.

In some embodiments. CD8⁺ cells are further sorted into naive, central memory, effector memory and effector cells by identifying cell surface antigens that are associated with each of these types of CD8⁺ cells. In certain embodiments. CD4⁺ T cells are further sorted into subpopulations. For example, CD4⁺ T helper cells can be sorted into naive, central memory, and effector cells by identifying cell populations that have cell surface antigens.

In some embodiments, the immune cells, e.g., T cells, are genetically modified following isolation using known methods, or the immune cells are activated and expanded (or differentiated in the case of progenitors) in vitro prior to being genetically modified. In another embodiment, the immune cells, e.g., T cells, are genetically modified with the chimeric antigen receptors described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874:6.867.041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads& system, a CD3/CD28 activator/stimulator system for physiological activation of human T cells. In other embodiments, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

### IV. Gene Delivery and Cell Modification

Expression cassettes included in vectors useful in the present disclosure contain (in a 5′-to-3′ direction) a transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation.

### (i) Promoter/Enhancers

The expression constructs provided herein comprise a promoter to drive expression of the CAR. A promoter used in the context of the present disclosure includes constitutive, inducible, and tissue-specific promoters. A promoter generally comprises a sequence that functions to position the start site for RNA synthesis. Additional promoter elements regulate the frequency of transcriptional initiation. The spacing between promoter elements frequently is flexible, so that promoter function is preserved when elements are inverted or moved relative to one another.

Non-limiting examples of promoters include early or late viral promoters, such as, SV40 early or late promoters, cytomegalovirus (CMV) immediate early promoters, Rous Sarcoma Virus (RSV) early promoters: eukaryotic cell promoters, such as, e.g., beta actin promoter, GADPH promoter, metallothionein promoter; and concatenated response element promoters, such as cyclic AMP response element promoters (ere), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is EF1, EF1 alpha. MND, CMV IE, dectin-1, dectin-2, human CDI 1c, F4/80, SM22, RSV, SV40, Ad MLP, beta-actin, MHC class I, MHC class II promoter, U6 promoter or HI promoter, however any other promoter that is useful to drive expression of the therapeutic gene is applicable to the practice of the present disclosure.

### (ii) Initiation Signals and Linked Expression

A specific initiation signal also may be used in the expression constructs provided in the present disclosure for efficient translation of coding sequences. These signals include the ATG initiation codon or adjacent sequences. One of ordinary skill in the art would readily be capable of determining this and providing the necessary signals.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements can be linked to heterologous open reading frames. Multiple open reading frames can be transcribed together, each separated by an IRES, creating polycistronic messages.

Additionally, certain 2A sequence elements could be used to create linked- or co-expression of genes in the constructs provided in the present disclosure. For example, cleavage sequences could be used to co-express genes by linking open reading frames to form a single cistron. Exemplary cleavage sequences include but are not limited to T2A. P2A. E2A and F2A. In a preferred embodiment, the cleavage sequence comprises a P2A sequence.

In some embodiments. P2A comprises an amino acid sequence having at least 90%, 91%, 92%, 93%, 94%, 95%, 96%, 97%, 98%, 99% or 100% identity with the amino acid sequence of GSGATNFSLLKQAGDVEENPGP (SEQ ID NO: 34).

(iii) Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively, a replication origin of other extra-chromosomally replicating virus as described above or an autonomously replicating sequence (ARS) can be employed.

### Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers.

In addition to markers conferring a phenotype that allows for the discrimination of transformants based on the implementation of conditions, other types of markers including screenable markers such as GFP, whose basis is colorimetric analysis, are also contemplated. In some embodiments, the reporter genes such as tEGFR are used. Further examples of selection and screenable markers are well known to one of skill in the art. One of skill in the art would also know how to employ immunologic markers, possibly in conjunction with FACS analysis. The marker used is not believed to be important, so long as it is capable of being expressed simultaneously with the nucleic acid encoding a gene product.

Cells may be modified to express the modified receptors described herein by any suitable method known in the art or described herein, for example, electroporation or lipofection. In some embodiments of the methods of the disclosure, introducing a nucleic acid sequence and/or a genomic editing construct into an immune cell ex vivo, in vivo, in vitro or in situ comprises a viral vector. In some embodiments of the methods of the disclosure, introducing a nucleic acid sequence and/or a genomic editing construct into an immune cell ex vivo, in vivo, in vitro or in situ comprises a combination of vectors. Exemplary, non-limiting vector combinations include: viral and non-viral vectors, a plurality of non-viral vectors, or a plurality of viral vectors. Exemplary but non-limiting vectors combinations include: a combination of a DNA-derived and an RNA-derived vector, a combination of an RNA and a reverse transcriptase, a combination of a transposon and a transposase, a combination of a non-viral vector and an endonuclease, and a combination of a viral vector and an endonuclease.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence, transiently integrates a nucleic acid sequence, produces site-specific integration a nucleic acid sequence, or produces a biased integration of a nucleic acid sequence. In some embodiments, the nucleic acid sequence is a transgene.

In some embodiments of the methods of the disclosure, genome modification comprising introducing a nucleic acid sequence and/or a genomic editing construct into an immune cell ex vivo, in vivo, in vitro or in situ stably integrates a nucleic acid sequence. In some embodiments, the stable chromosomal integration can be a random integration, a site-specific integration, or a biased integration. In some embodiments, the site-specific integration can be non-assisted or assisted. In some embodiments, the assisted site-specific integration is co-delivered with a site-directed nuclease. In some embodiments, the site-directed nuclease comprises a transgene with 5′ and 3′ nucleotide sequence extensions that contain a percentage homology to upstream and downstream regions of the site of genomic integration. In some embodiments, the transgene with homologous nucleotide extensions enable genomic integration by homologous recombination, microhomology-mediated end joining, or nonhomologous end-joining. In some embodiments the site-specific integration occurs at a safe harbor site. Genomic safe harbor sites are able to accommodate the integration of new genetic material in a manner that ensures that the newly inserted genetic elements function reliably (for example, are expressed at a therapeutically effective level of expression) and do not cause deleterious alterations to the host genome that cause a risk to the host organism. Potential genomic safe harbors include, but are not limited to, intronic sequences of the human albumin gene, the adeno-associated virus site 1 (AAVS1), a naturally occurring site of integration of AAV virus on chromosome 19, the site of the chemokine (C-C motif) receptor 5 (CCR5) gene and the site of the human ortholog of the mouse Rosa26 locus.

In some embodiments, the site-specific transgene integration occurs at a site that disrupts expression of a target gene. In some embodiments, disruption of target gene expression occurs by site-specific integration at introns, exons, promoters, genetic elements, enhancers, suppressors, start codons, stop codons, and response elements. In some embodiments, exemplary target genes targeted by site-specific integration include but are not limited to any immunosuppressive gene, and genes involved in allo-rejection.

In some embodiments, the site-specific transgene integration occurs at a site that results in enhanced expression of a target gene. In some embodiments, enhancement of target gene expression occurs by site-specific integration at introns, exons, promoters, genetic elements, enhancers, suppressors, start codons, stop codons, and response elements.

In addition to viral delivery of the nucleic acids encoding the antigen receptor, the following are additional methods of recombinant gene delivery to a given cell. (e.g. an NK cell) and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection): by electroporation: by calcium phosphate precipitation: by using DEAE-dextran followed by polyethylene glycol: by direct sonic loading: by liposome mediated transfection and receptor-mediated transfection: by microprojectile bombardment: by agitation with silicon carbide fibers: by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

In some embodiments of the methods of the disclosure, introducing a nucleic acid sequence and/or a genomic editing construct into an immune cell ex vivo, in vivo, in vitro or in situ comprises a non-viral vector. In some embodiments, the non-viral vector comprises a nucleic acid. In some embodiments, the non-viral vector comprises plasmid DNA, linear double-stranded DNA (dsDNA), linear single-stranded DNA (ssDNA). Doggy Bone™ DNA, nanoplasmids, minicircle DNA, single-stranded oligodeoxynucleotides (ssODN), DDNA oligonucleotides, single-stranded mRNA (ssRNA), and double-stranded mRNA (dsRNA). In some embodiments, the non-viral vector comprises a transposon of the disclosure.

In some embodiments of the methods of the disclosure, enzymes may be used to create strand breaks in the host genome to facilitate delivery or integration of the transgene. In some embodiments, enzymes create single-strand breaks. In some embodiments, enzymes create double-strand breaks. In some embodiments, examples of break-inducing enzymes include but are not limited to: transposases, integrases, endonucleases, meganucleases, megaTALs, CRISPR-Cas9, CRISPR-CasX, transcription activator-like effector nucleases (TALEN) or zinc finger nucleases (ZFN). In some embodiments, break-inducing enzymes can be delivered to the cell encoded in DNA, encoded in mRNA, as a protein, as a nucleoprotein complex with a guide RNA (gRNA).

### V. Methods of Use

In some embodiments, the present disclosure provides methods for immunotherapy comprising administering an effective amount of the immune cells (e.g. engineered T-cells) of the present disclosure. In one embodiment, a medical disease or disorder is treated by transfer of an immune cell population that elicits an immune response. In certain embodiments of the present disclosure, autoimmune, alloimmune, allergic, or malignant disease is treated by transfer of an immune cell population that elicits an immune response.

Provided herein are methods for treating or delaying progression of autoimmune disease, transplant rejection and/or chronic inflammatory disease in an individual comprising administering to a subject an effective amount an antigen-specific cell therapy (e.g. engineered T-cells of the disclosure). Diseases for which the present treatment methods are useful include any diseases wherein a pathologic or pathogenic T-cell type is present in the subject. In some embodiments, the pathologic or pathogenic T-cell is a pathologic or pathogenic CD8+ T-cell.

Administration of therapeutically effective amount of a pharmaceutical composition comprising the engineered T-cells of the disclosure to a subject under conditions suitable for binding of the engineered T-cells to the pathologic T cells (e.g. CD8+ pathologic T cells) of a subject, results in cell death of the population of pathologic T cells in the subject.

In some embodiments, the cell death of the population of CD8+ pathologic T cells in the subject is about 2 fold to about 100 fold higher than the cell death of a population of CD8+ pathologic T cells in a subject that has not been administered with the pharmaceutical composition comprising the engineered T cells. In some embodiments, the cell death of the population of CD8+ pathologic T cells in the subject is about 2 fold to about 25 fold higher than the cell death of a population of CD8+ pathologic T cells in a subject that has not been administered with the pharmaceutical composition comprising the engineered T cells. In some embodiments, the cell death of the population of CD8+ pathologic T cells in the subject is about 2 fold, about 3 fold, about 4 fold, about 5 fold, about 6 fold, about 7 fold about 8 fold, about 9) fold or about 10 fold higher than the cell death of a population of CD8+ pathologic T cells in a subject that has not been administered with the pharmaceutical composition comprising the engineered T cells.

Common autoimmune disorders include type 1 diabetes mellitus, systemic lupus erythematosus, psoriasis and psoriatic arthritis, rheumatoid arthritis. (Hashimoto's) autoimmune thyroiditis, inflammatory bowel diseases such as ulcerative colitis and Crohn's disease, autoimmune hepatitis, primary biliary cholangitis, pernicious anemia. Celiac disease, autoimmune vasculitis. Sjogren's disease, and multiple sclerosis.

In some embodiments, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia. Bechet's disease, bullous pemphigoid, cardiomyopathy, celiac spate-dermatitis, chronic fatigue immune dysfunction syndrome (CFIDS), chronic inflammatory demyelinating polyneuropathy. Churg-Strauss syndrome, cicatrical pemphigoid. CREST syndrome, cold agglutinin disease. Crohn's disease, discoid lupus, essential mixed cryoglobulinemia, fibromyalgia-fibromyositis, glomerulonephritis. Graves' disease. Guillain-Barre. Hashimoto's thyroiditis, idiopathic pulmonary fibrosis, idiopathic thrombocytopenia purpura (ITP). IgA neuropathy, juvenile arthritis, lichen planus, lupus erythematosus. Meniere's disease, mixed connective tissue disease, multiple sclerosis, type 1 or immune-mediated diabetes mellitus, myasthenia gravis, nephrotic syndrome (such as minimal change disease, focal glomerulosclerosis, or membranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis. Raynaud's phenomenon. Reiter's syndrome. Rheumatoid arthritis, sarcoidosis, scleroderma (systemic sclerosis). Sjogren's syndrome, stiff-man syndrome, systemic lupus erythematosus, lupus erythematosus, ulcerative colitis, uveitis, vasculidites (such as polyarteritis nodosa. Takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosus, type I diabetes mellitus. Crohn's disease: ulcerative colitis, myasthenia gravis, glomerulonephritis, ankylosing spondylitis, vasculitis, or psoriasis. The subject can also have an allergic disorder such as asthma, chronic beryllium disease. Celiac's disease, atopic eczema, or certain drug allergies.

In some embodiments, the subject has a chronic inflammatory or metabolic disease. Non-limiting examples of chronic inflammatory or metabolic disease include: hypertension, cardiovascular disease, non-alcoholic fatty liver disease (steatotic liver disease).

In some embodiments, the subject has a T cell mediated malignancy. Non-limiting examples of T cell mediated malignancy include: T cell leukemias. T cell lymphomas including cutaneous T cell lymphoma (CTCL), enteropathy-associated T cell lymphoma (EATL), anaplastic large cell lymphoma (ALCL), peripheral T cell lymphoma—not otherwise specified (PTCL-NOS), angioimmunoblastic T-cell lymphoma (AITL).

In some embodiments, the subject is the recipient of a transplanted organ or stem cells and immune cells are used to prevent and/or treat rejection. Transplant rejection can be acute, sub-acute or chronic. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash on the hands and feet that may spread and become more severe, with peeling or blistering skin. Acute GVHD can also affect the stomach and intestines, in which case cramping, nausea, and diarrhea are present. Yellowing of the skin and eyes (jaundice) indicates that acute GVHD has affected the liver. Chronic GVHD is ranked based on its severity: stage/grade 1 is mild: stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines.

Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least l day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

Therapeutically effective amounts of immune cells can be administered by a number of routes, including parenteral administration, for example, intravenous, intraperitoneal, intramuscular, intrasternal, or intraarticular injection, or infusion.

The therapeutically effective amount of immune cells for use in adoptive cell therapy is that amount that achieves a desired effect in a subject being treated. For instance, this can be the amount of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several weeks to ameliorate a disease state or periodic doses over an extended time to inhibit disease progression and prevent disease recurrence. The precise dose to be employed in the formulation will also depend on the route of administration, and the seriousness of the disease or disorder. The therapeutically effective amount of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as from about 2×10⁷ cells to about 5×10⁸ cells per kg body weight, or from about 5×10⁷ cells to about 2×10⁸ cells per kg body weight, or from about 5×10⁶ cells per kg body weight to about 1×10⁷ cells per kg body weight. In some embodiments, a therapeutically effective amount of immune cells ranges from about 1×10⁵ cells per kg body weight to about 10×10⁹ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

### 1. Pharmaceutical Compositions

Also provided herein are pharmaceutical compositions and formulations comprising immune cells (e.g., T cells) and a pharmaceutically acceptable carrier.

In some embodiments, a pharmaceutical composition comprises a dose ranging from about 1×10⁵ cells to about 1×10⁹ cells. In some embodiments, the dose is about 1×10⁵, 1×10⁶, 1×10⁷, 1×10⁸ or 1×10⁹ cells. In some embodiments, a pharmaceutical composition comprises a dose ranging from about 5×10⁵ cells to about 10×10¹² cells.

In some embodiments, a pharmaceutical composition comprises a population of cells comprising about 1×10⁵ cells to about 1×10⁹ cells. In some embodiments, the pharmaceutical composition comprises a population of cells comprising about 1×10⁵, 1×10⁶, 1×10⁷, 1×10⁸ or 1×10⁹ cells. In some embodiments, a pharmaceutical composition comprises a population of about 5×10⁵ cells to about 10×10¹² cells.

Pharmaceutical compositions and formulations as described herein can be prepared by mixing the active ingredients (such as an antibody or a polypeptide) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids: antioxidants including ascorbic acid and methionine: preservatives (such as octadecyldimethylbenzyl ammonium chloride: hexamethonium chloride: benzalkonium chloride: benzethonium chloride: phenol, butyl or benzyl alcohol: alkyl parabens such as methyl or propyl paraben: catechol: resorcinol; cyclohexanol: 3-pentanol; and m-cresol): low molecular weight (less than about 10 residues) polypeptides: proteins, such as serum albumin. gelatin, or immunoglobulins: hydrophilic polymers such as polyvinylpyrrolidone: amino acids such as glycine, glutamine, asparagine, histidine, arginine, or lysine: monosaccharides, disaccharides, and other carbohydrates including glucose, mannose, or dextrins: chelating agents such as EDTA: sugars such as sucrose, mannitol, trehalose or sorbitol: salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEXR. Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### 2. Combination Therapies

In some embodiments, the compositions and methods of the present embodiments involve an immune cell population in combination with at least one additional therapy. The additional therapy may be radiation therapy, surgery (e.g., lumpectomy and a mastectomy), chemotherapy, gene therapy. DNA therapy, viral therapy. RNA therapy, immunotherapy, bone marrow transplantation, nanotherapy, monoclonal antibody therapy, or a combination of the foregoing. The additional therapy may be in the form of adjuvant or neoadjuvant therapy.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine) immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokine antagonists (for example, anti-TNF agents such as infliximab, adalimumab, golimumab, natalizumab, anti-IL-6 such as tocilizumab and sarilizumab, anti-12/23 such as ustekinumab), cytokines (for example, interleukin-10 or transforming growth factor-beta), anti-trafficking agents (for example, anti-integrins such as vedolizumab and SIP inhibitors such as ozanimod, etrasimod, and fingolomid), hormones (for example, estrogen), or a vaccine. In addition, immunosuppressive or tolerogenic agents including but not limited to anti-thymocyte globulin, calcineurin inhibitors (e.g., cyclosporin and tacrolimus): mTOR inhibitors (e.g., rapamycin, sirolimus): mycophenolate mofetil, antibodies (e.g., recognizing CD3, CD4, CD40, CD154, CD45, IVIG, or B cells): chemotherapeutic agents (e.g., methotrexate, treosulfan, busulfan): irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### VI. Dosage Regimens

In one embodiment, the immune effector cells (e.g., T cells) are modified by engineering/introducing chimeric antigen receptors into said immune effector cells and then infused into a subject. In some embodiments, immune effector cells are modified by engineering/introducing a chimeric receptor, and functional effector element and/or a cytokine into the immune effector cells and then infused within about 0 days, within about 1 day, within about 2 days, within about 3 days, within about 4 days, within about 5 days, within about 6 days or within about 7 days into a subject.

In some embodiments, an amount of modified effector cells is administered to a subject in need thereof and the amount is determined based on the efficacy and the potential of inducing a cytokine-associated toxicity. In another embodiment, the modified effector cells are CAR⁺ and CD56⁺ cells. In some embodiments, an amount of modified effector cells comprises about 10⁴ to about 10⁹ modified effector cells/kg. In some cases, an amount of modified effector cells comprises about 10⁴ to about 10⁵ modified effector cells/kg. In some cases, an amount of modified effector cells comprises about 10⁵ to about 10⁶ modified effector cells/kg. In some cases, an amount of modified effector cells comprises about 10⁶ to about 10⁷ modified effector cells/kg. In some cases, an amount of modified effector cells comprises about 10⁷ to about 10⁸ modified effector cells/kg. In some cases, an amount of modified effector cells comprises about 10⁸ to about 10⁹ modified effector cells/kg. In some cases, am amount of modified effector cells comprises about 0.1×10⁶, about 0.2×10⁶, about 0.3×10⁶, about 0.4×10⁶, about 0.5×10⁶, about 0.6×10⁶, about 0.7×10⁶, about 0.8×10⁶, about 0.9×10⁶, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹ modified effector cells/kg.

In one embodiment, the modified immune effector cells are targeted to the cancer via regional delivery directly to the tumor tissue. For example, in ovarian or renal cancer, the modified immune effector cells can be delivered intraperitoneally (IP) to the abdomen or peritoneal cavity. Such IP delivery can be performed via a port or pre-existing port placed for delivery of chemotherapy drugs. Other methods of regional delivery of modified immune effector cells can include catheter infusion into resection cavity, ultrasound guided intra-tumoral injection, hepatic artery infusion or intrapleural delivery.

In one embodiment, a subject in need thereof, can begin therapy with a first dose of modified immune effector cells delivered via IV followed by a second dose of modified immune effector cells delivered via IV. In one embodiment, a subject in need thereof, can begin therapy with a first dose of modified immune effector cells delivered via IP followed by a second dose of modified immune effector cells delivered via IV. In a further embodiment, the second dose of modified immune effector cells can be followed by subsequent doses which can be delivered via IV or IP. In one embodiment, the duration between the first and second or further subsequent dose can be about: 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 days. In one embodiment, the duration between the first and second or further subsequent dose can be about: 0), 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 months. In some embodiments, the duration between the first and second or further subsequent dose can be about: 0, 1, 2, 3, 4, 5, 6, 7, 8, 9 or 10 years.

In another embodiment, a catheter can be placed at the tumor or metastasis site for further administration of 1, 2, 3, 4, 5, 6, 7, 8, 9, 10 doses of modified immune effector cells. In some cases, doses of modified effector cells can comprise about 10² to about 10⁹ modified effector cells/kg. In cases where toxicity is observed, doses of modified effector cells can comprise about 10² to about 10⁵ modified effector cells/kg. In some cases, doses of modified effector cells can start at about 10² modified effector cells/kg and subsequent doses can be increased to about: 10⁴, 10⁵, 10⁶, 10⁷, 10⁸ or 10⁹ modified effector cells/kg.

### VII. Articles of Manufacture or Kits

An article of manufacture or a kit is provided comprising immune cells is also provided herein. The article of manufacture or kit can further comprise a package insert comprising instructions for using the immune cells to treat or delay progression of cancer in an individual or to enhance immune function of an individual having cancer. Any of the antigen-specific immune cells described herein may be included in the article of manufacture or kits. Suitable containers include, for example, bottles, vials, bags and syringes. The container may be formed from a variety of materials such as glass, plastic (such as polyvinyl chloride or poly olefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent. Suitable containers for the one or more agent include, for example, bottles, vials, bags and syringes.

### VIII. Examples

### Example 1: Precision Killing of T Cells Via Targeting of T Cell Receptors Using Novel CAR-T Cells

Pathologic T cells drive T cell leukemia, solid organ transplant rejection, and many autoimmune diseases. Currently approved treatments have limited ability to target pathogenic vs non-pathogenic T cells. While pan-T cell treatments can be effective, they have significant risks such as infection, metabolic disease, cardiovascular disease and malignancy. Chimeric antigen receptors (CAR) are synthetic constructs that retarget T cells to a specific antigen of interest: we hypothesized that we could use this technology for targeted killing of T cells.

Two novel CAR designs which target T cells via their T cell receptor (TCR) were constructed. These CAR-T cells target OT-I T cells, a model system with a known antigenic epitope. The anti-Vβ5 CAR is a second-generation CAR with a traditional extracellular scFv domain that targets Vβ5 of the OT-I TCR. The H2-K^{b} bait CAR (“OVA-MHCI Bait-SIINFEKL”CAR) instead has an extracellular H2-K^{b} complex loaded with the SIINFEKL cognate epitope. Both anti-Vβ5 and H2-K^{b} bait CAR T cells demonstrate degranulation, activation, and proliferation upon binding OT-I T cells. In vitro co-culture with OT-I T cells results in internalization of the OT-I TCR and specific killing of OT-I T cells. The changes induced by the H2-K^{b} bait CAR were delayed relative to the anti-Vβ5 CAR. In vivo experiments show both CAR designs kill adoptively transferred OT-I T cells without significant effect on the other T cell populations.

In conclusion, two novel CAR designs which allow for the targeted killing of T cells via their T cell receptor were developed. While the anti-Vβ5 CAR T cells generated a more rapid response, the H2-K^{b} bait CAR offers the promise of more precisely targeting T cells. This work has the potential to transform the treatment of conditions where removal of pathogenic T cells is critical for cure.

### Example 2: MHC Class I (MHC-1) Bait CAR Design and Expression

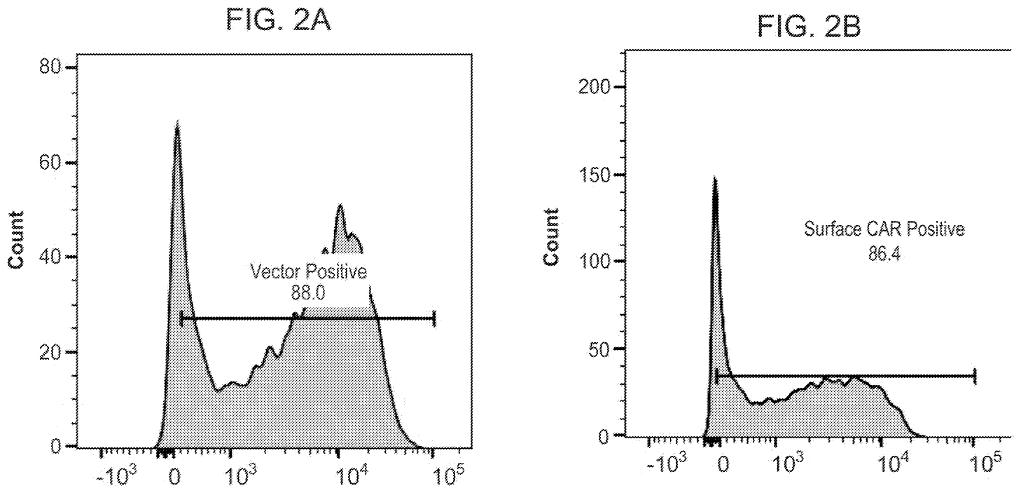

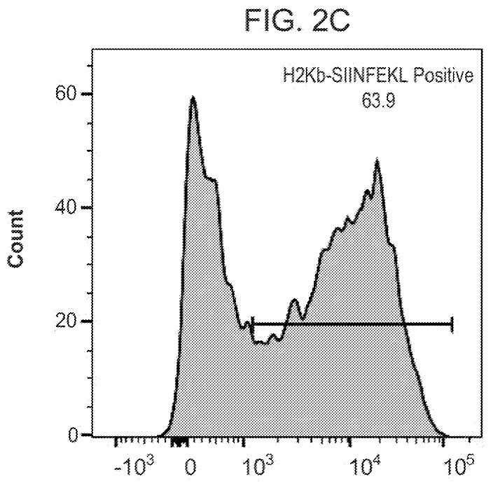

A nucleotide sequence encoding an exemplary MHC-1 bait CAR (e.g. “OVA-MHCI Bait-SIINFEKL” CAR) was transduced into T cells and surface expression of the exemplary CAR was determined using flow cytometry. Surface expression of multiple CAR components was analyzed including EGFR (reporter gene) (FIG. 2A). G4S (linker domain) (FIG. 2B) and MHC Class I (H2-K^{b}) containing exemplary peptide bait SIINFEKL (FIG. 2C). Surface expression was detected using antibodies and the proportion of the cell population expressing each component was quantified using flow cytometry. FIG. 2A shows that about 88% of the T cell population expressed the EGFR reporter demonstrating transduction efficiency. FIG. 2B shows that about 86.4% of cells were positive for surface expression of the G4S CAR subunit. FIG. 2C shows that about 63.9% of cell population were H2Kb-SIINFEKL positive (MHC-1 plus bait epitope).

### Example 3: MHC-I Bait CAR In Vitro Profile Vs Monoclonal Target T Cells

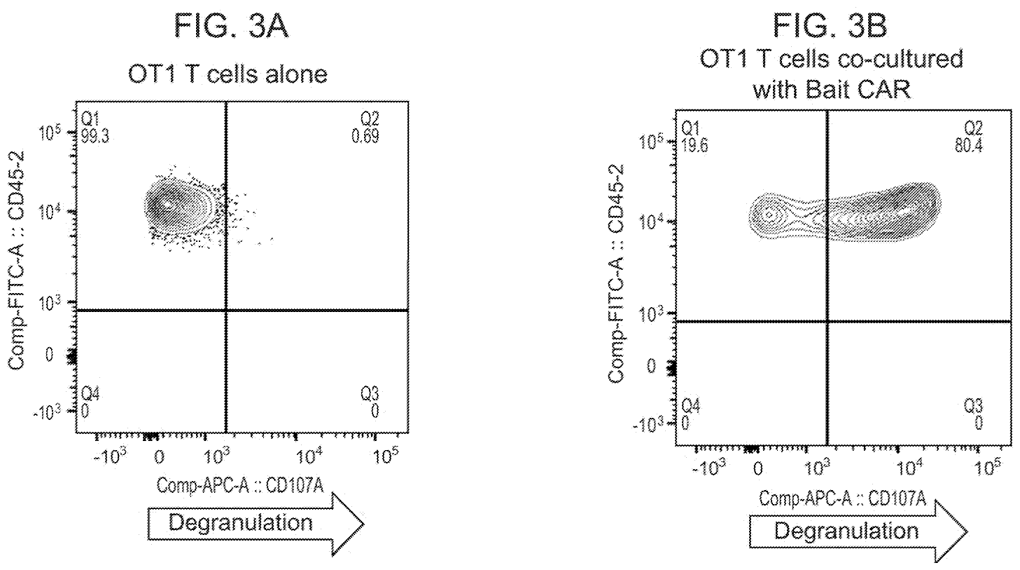

T cells expressing exemplary MHC-1 bait CARs were cocultured with OT-I target T cells to assess recognition and activation of target T cells in vitro. The target cell population was identified and sorted using flow cytometry. CD107a was used as a surface marker for T cell activation. FIG. 3B demonstrates that exemplary MHC-I bait CAR T cells induce degranulation of OT-I T cells (about 80.4% of the cell population was CD107a-positive). Only about 0.69% of OT-I T cell population was activated in the absence of exemplary MHC-I bait CARs, as shown in FIG. 3A.

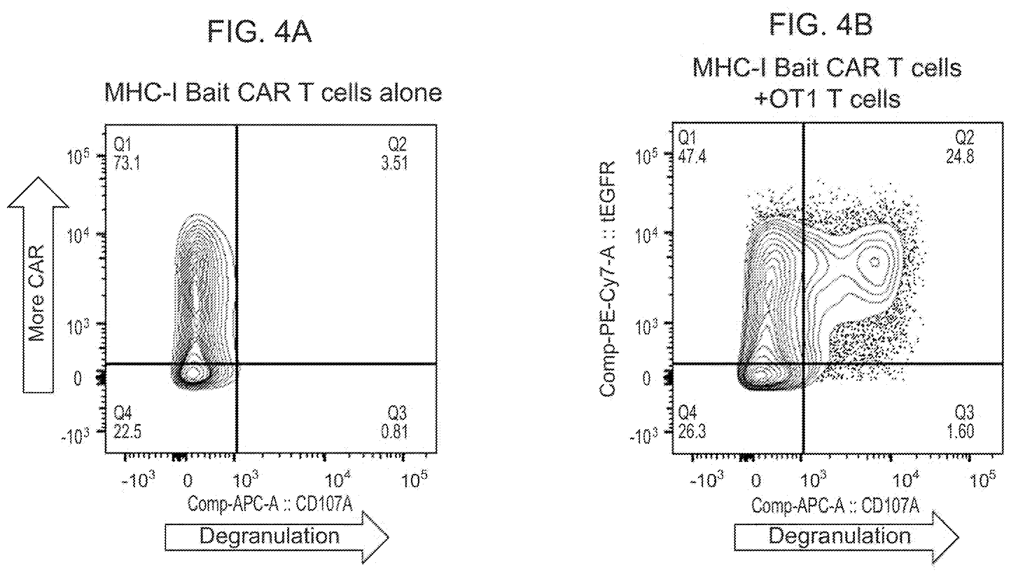

As shown in FIG. 4B. 24.8% of a population of exemplary MHC-I bait CAR T cell were activated (expression of CD107a) when incubated in the presence of the target OT-IT cells. This proportion was significantly higher than the number of exemplary MHC-I bait CAR T cells that were activated in the absence of OT-I target cells (about 3.51%), as shown in FIG. 4A.

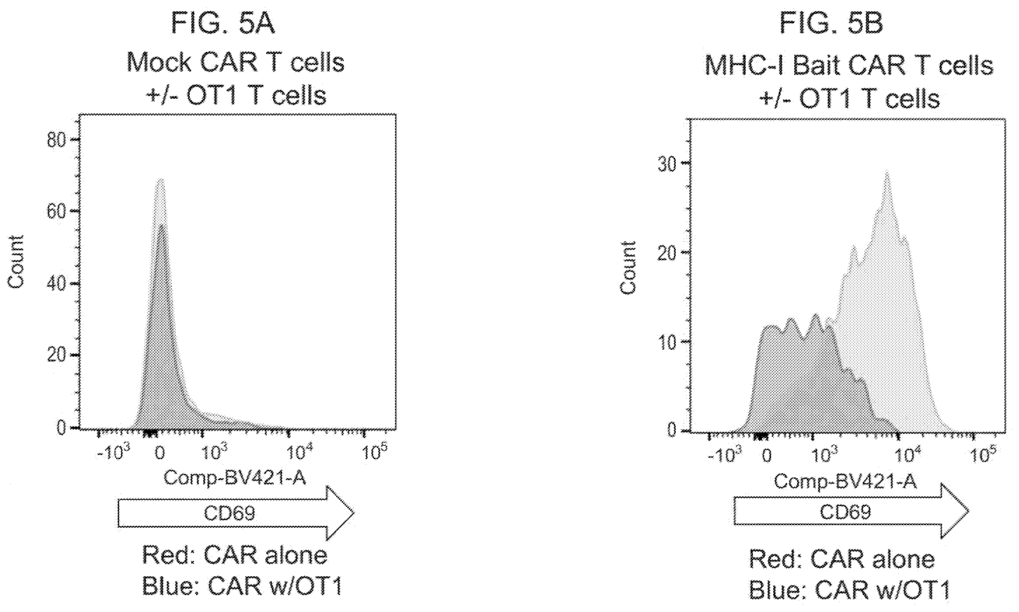

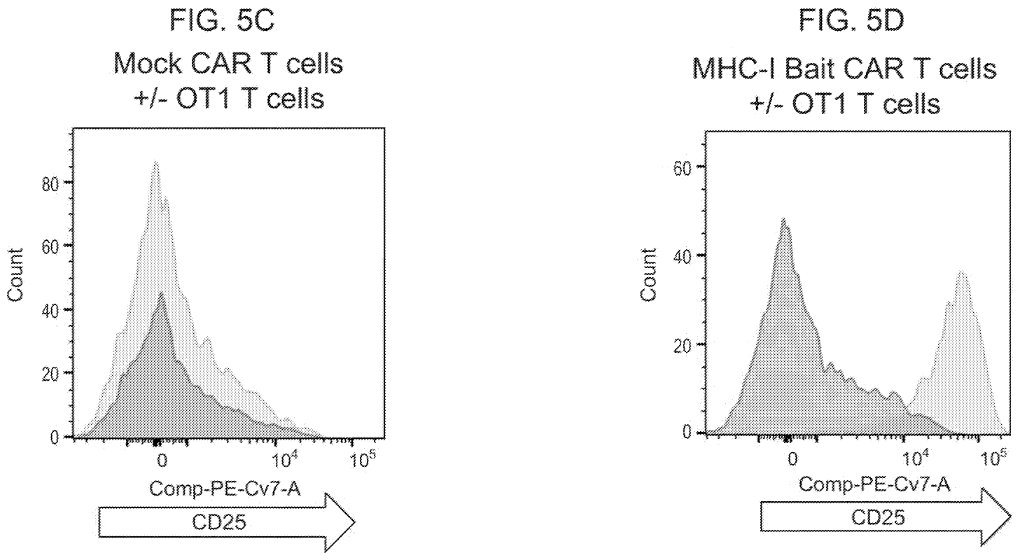

When incubated in the presence of target OT-I T cells, exemplary bait CAR T cells are express higher levels of CD69 (FIG. 5B) and CD25 (FIG. 5D) compared to expression of these markers in the absence of OT-I cells. A similar increase in CD69 (FIG. 5A) and CD25 (FIG. 5C) expression on mock CAR T cells is not seen in the presence of OT-I cells and is indistinguishable from expression levels in the absence of OT-I cells.

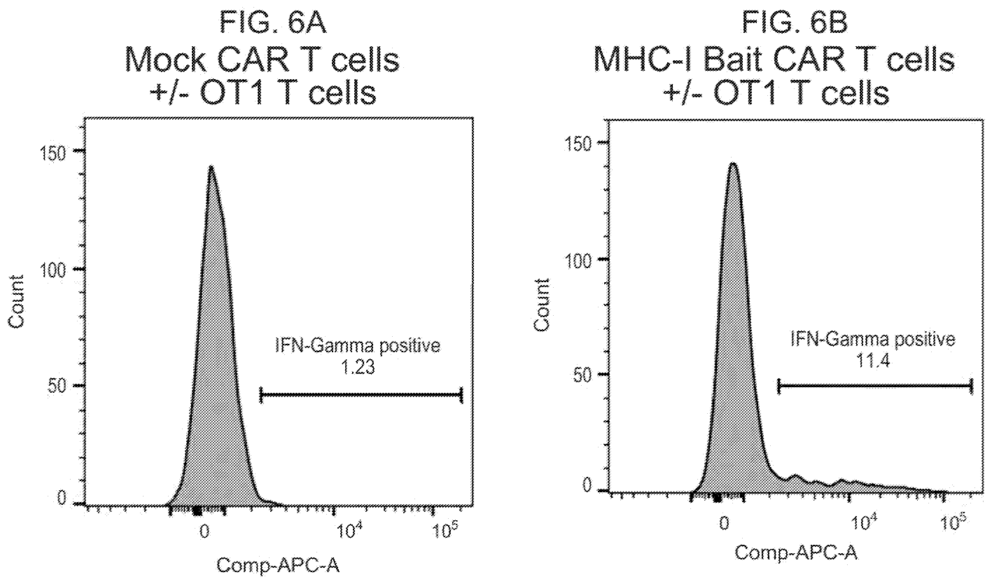

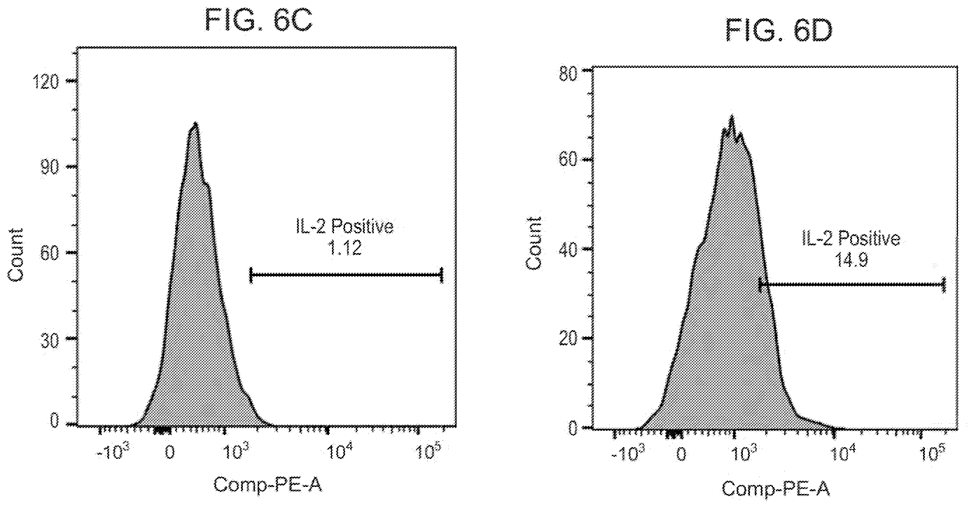

MHC-I bait CAR T cells generate cytokines when co-cultured in the presence of target OT-I cell in vitro. A larger proportion of exemplary MHC-I bait CAR T cells are IFN-gamma positive (about 11.4%) (FIG. 6B) than mock CAR T cells (about 1.23%) (FIG. 6A) in the presence of OT-I target cells. A higher number of exemplary CAR T cells are IL-2 positive (about 14.9%) (FIG. 6D), than mock CAR T cells (about 1.12%) (FIG. 6A) in the presence of OT-I cells.

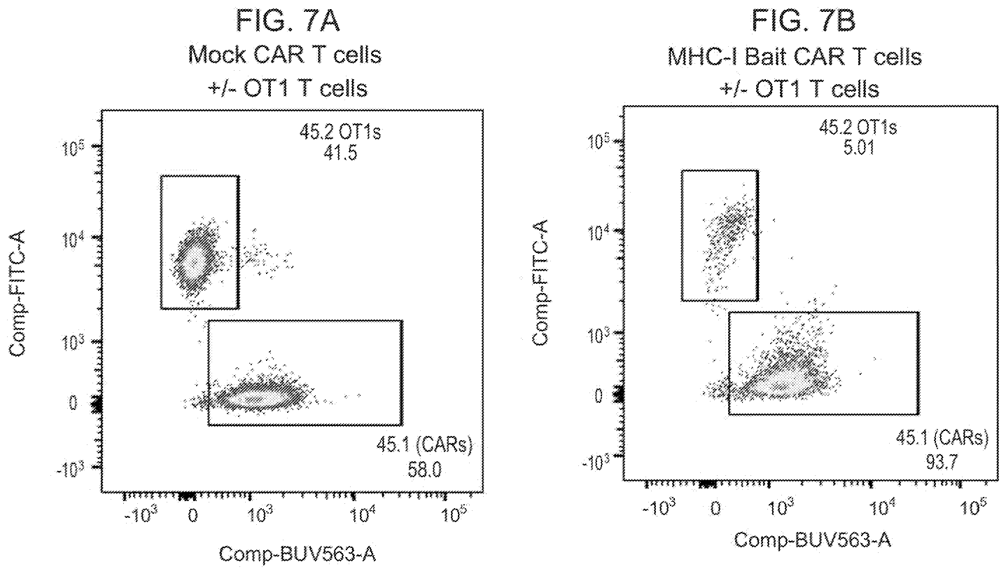

Competitive survival assays were conducted to monitor exemplary MHC-I bait CAR T cells and OT-I target cell survival. The two populations were cocultured together for 48 hours and the relative population of each cell type was quantified by flow cytometry. OT-I target cells were more readily killed by MHC-I bait CAR T cells than by mock CAR T cells. FIG. 7B shows that about 5.01% of the cell population comprised OT-I cells after incubation with exemplary MHC-1 bait CAR cells for 48 hours. FIG. 7A shows that OT-I cells comprised about 41.5% of the population of cells when cocultured in the presence of mock CAR T cells.

### Example 4: Functional Optimization of MHC-I Bait CAR

MHC-I bait CARs can be optimized via three methods: mutation of the CD8 binding site, mutation of the epitope (binding site/bait), and mutation of the ITAM domains. The optimization results in a balance of decreasing affinity/kinetics of interaction for the TCR while maintaining or increasing the affinity/kinetics of the CAR.

Two exemplary bait CDRs containing ITAM domain mutations were tested to assess the potency of the bait CAR toward OT-I target cells relative to mock CAR T cells potency. Survival of OT-I cells in coculture with bait CAR variants (mock, SCTI-Zi2. SCT1-Lx3 (“OVA-MHCI Bait-CD3Zeta Fully Live”)) was monitored over 60 hours of cell coculture. SCTI represents a wildtype MHCI (Q226. D227). Then, cell populations were quantified using flow cytometry to determine proportion of the cellular population of OT-I cells that survived in the presence of either mock T cells or ITAM variants. The proportion of OT-I cells was assessed after 18 hours. 36 hours and 60 hours of coculture with bait CAR T cell variants.

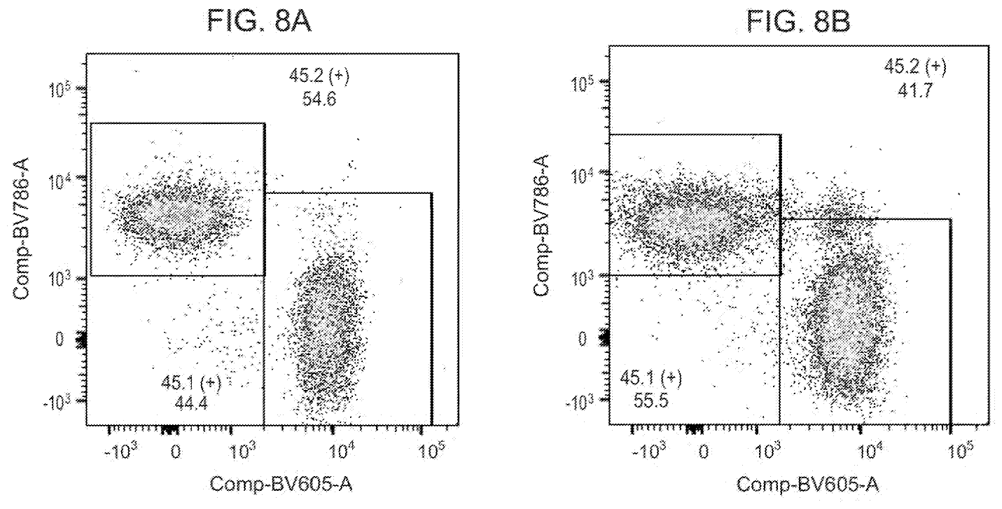

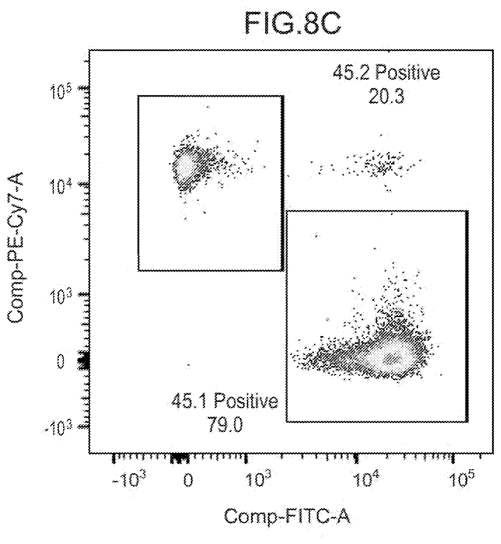

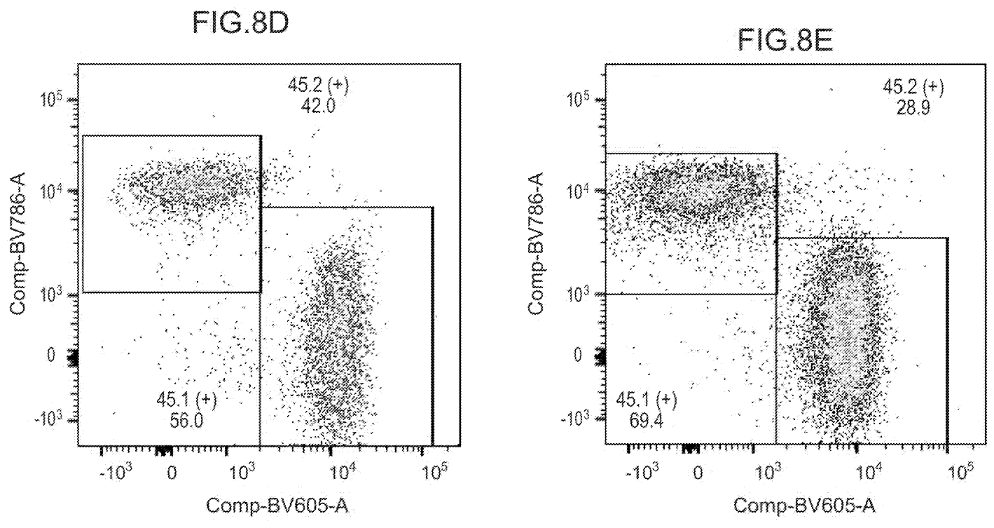

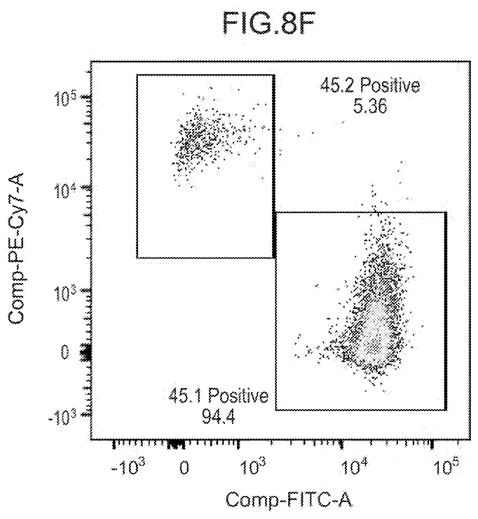

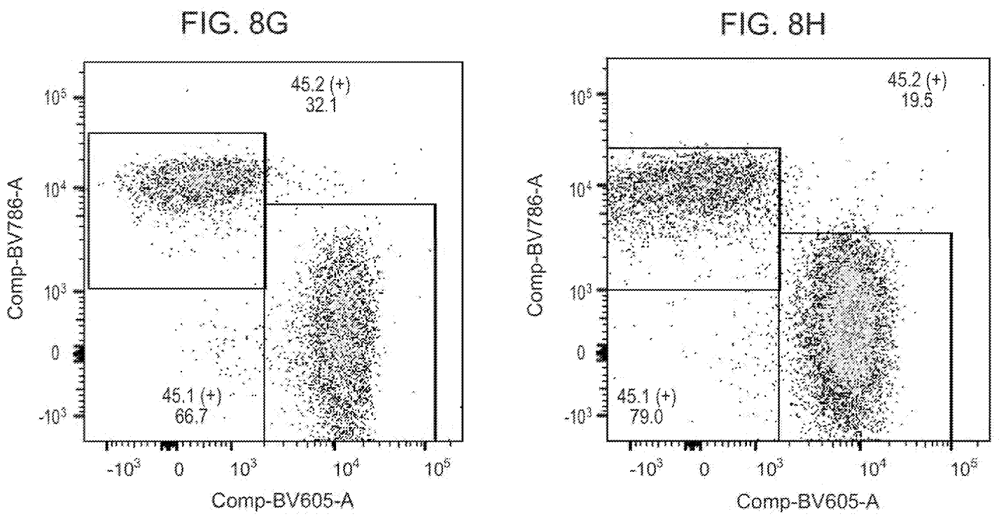

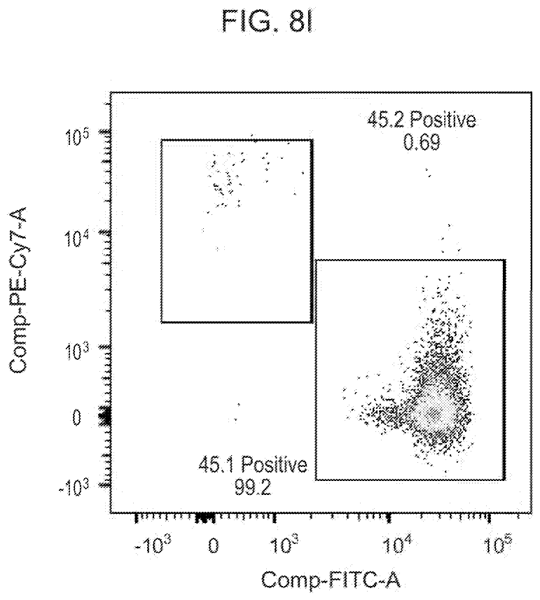

As shown in FIG. 8A-8I. MHC-I bait CAR T cells expressing CD3 ITAM mutant SCTI-Lx3 were more potent to OT-I target cells relative to mock CAR T cells. At 18 hours (about 32.1% vs about 54.6%). 36 hours (about 19.5% vs about 41.7%) and 60 hours (about 0.69% vs about 20.3%). MHC-I bait CAR T cells expressing CD3 ITAM mutant SCT1-Zi2 were more potent to OT-I T cells compared to mock T cells at all time points.

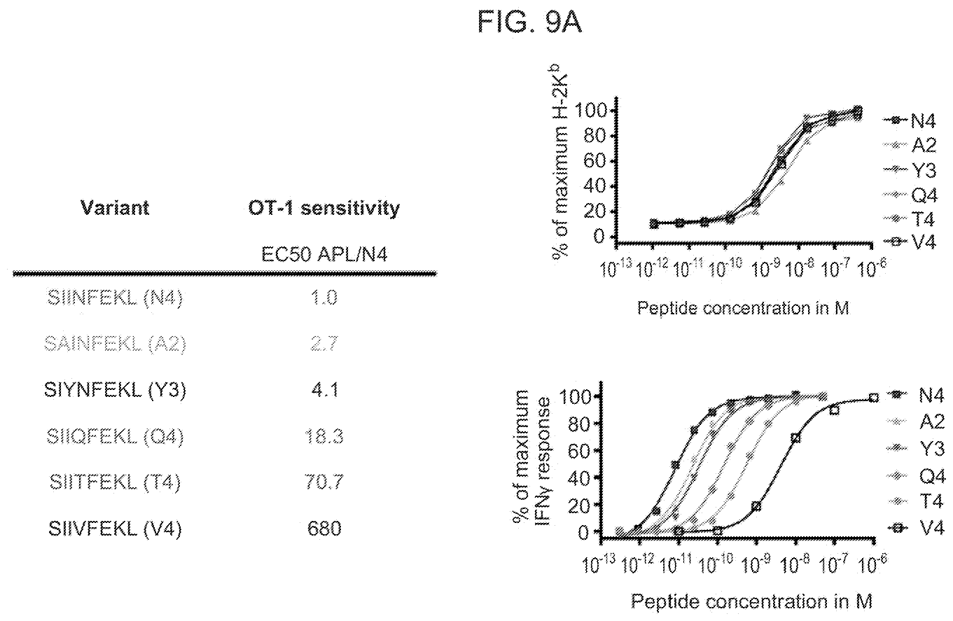

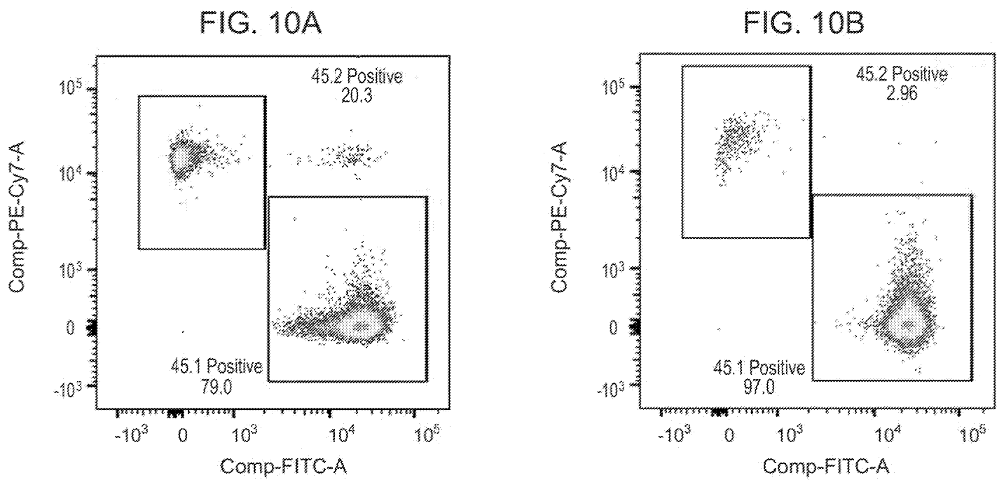

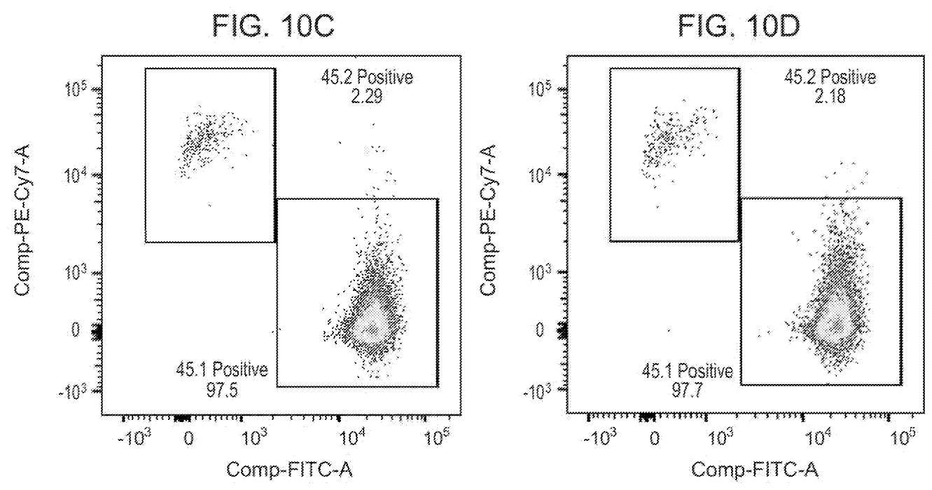

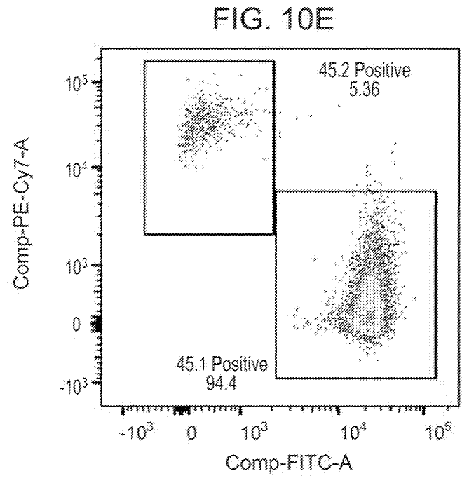

Mutations of OVA cognate peptides were designed to affect (target) OT-I activation without affecting binding affinity. OVA cognate peptide mutations and sequences are shown in Tables 1. Table 2 and FIG. 9C. Mutations to the peptide used in MHC-I Bait CARs can impact the activation of target OT-I cells. The mutation to these peptides confer survival advantage to bait CAR T cells. Mutations were introduced into bait CAR T cells expressing the SCT1-Zi2 CD3 ITAM mutation and exemplary bait CAR T cells were cocultured with OT-I target cells to monitor survival of both cell types. Each cell population was quantified by flow cytometry. Four mutant variants showed more potency toward OT-I target cells relative to mock T cells. FIG. 10A shows that the population of surviving OT-I cells was: about 20.3% with mock T cells. FIGS. 10B-E show that the population of surviving OT-I cells ranged between about 2.18% to about 5.36% with exemplary peptide mutant bait CARs. FIG. 10F shows the sensitivity of OT-I T cells to exemplary bait CAR variants.

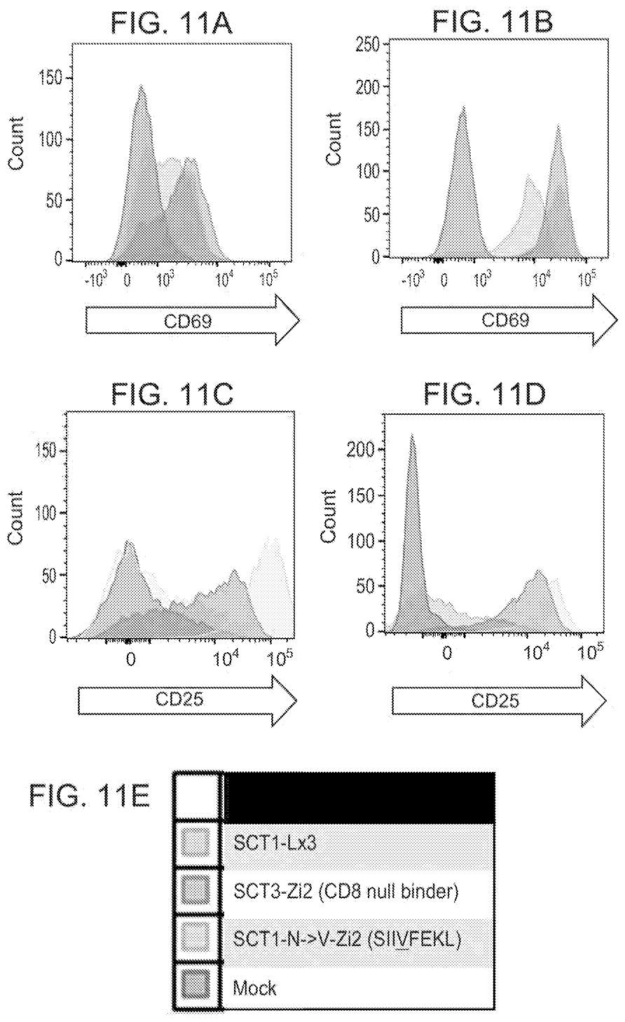

Mutations were introduced into the peptide and CD8 binding sites of the bait CARs. Exemplary variants identified in the legend of FIG. 11E containing peptide and CD8 binding mutations were assessed for their effect on the activation of target OT-I cells by monitoring expression of CD69 and CD25 on exemplary bait CAR T cells and OT-I T cells and quantified by flow cytometry. Exemplary bait CARs caused higher levels of CD69 and CD25 expression on OT-I cells and bait CAR T cells compared to expression induced by mock CAR T cells.

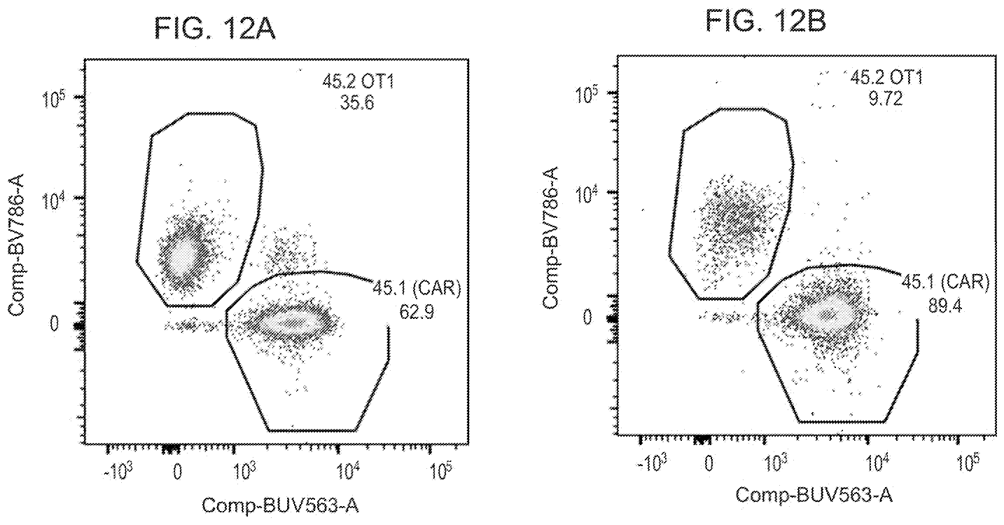

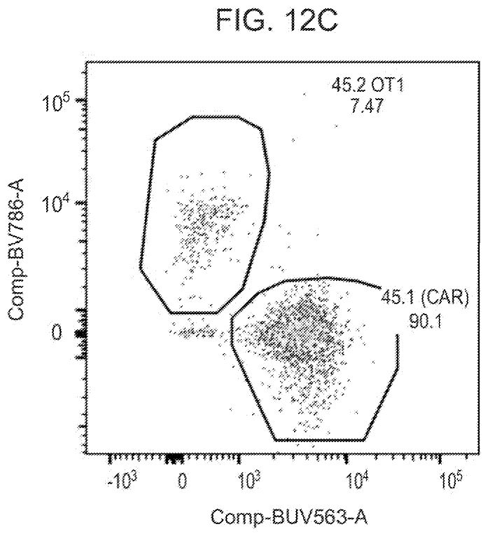

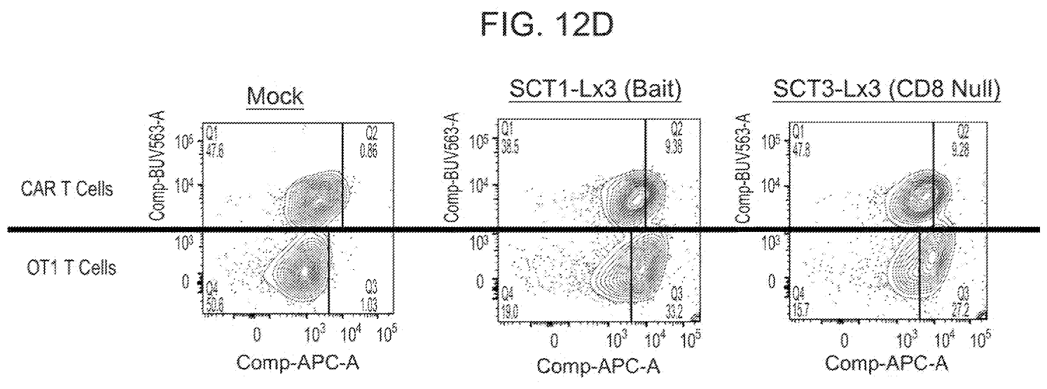

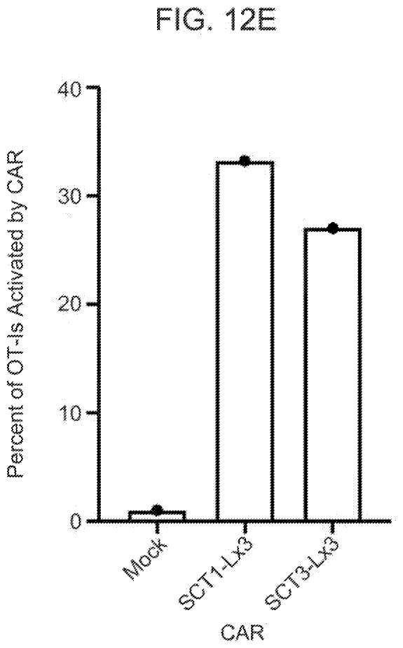

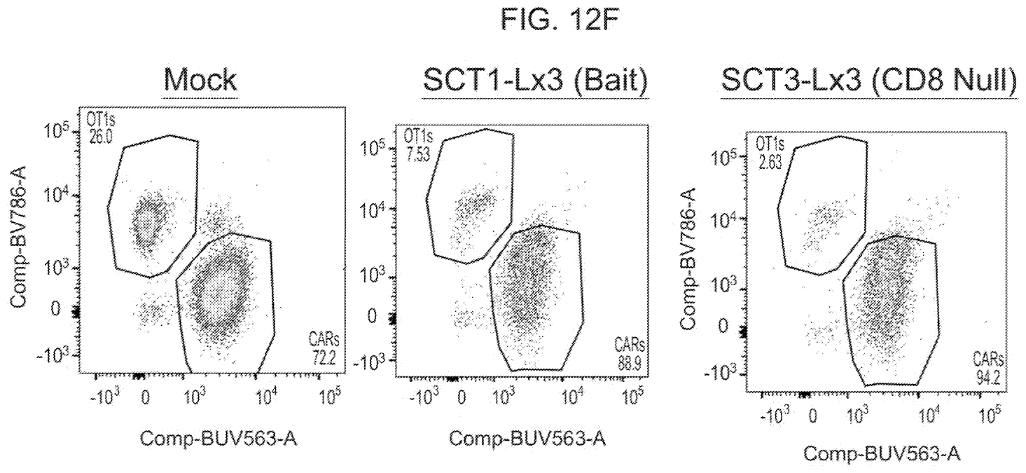

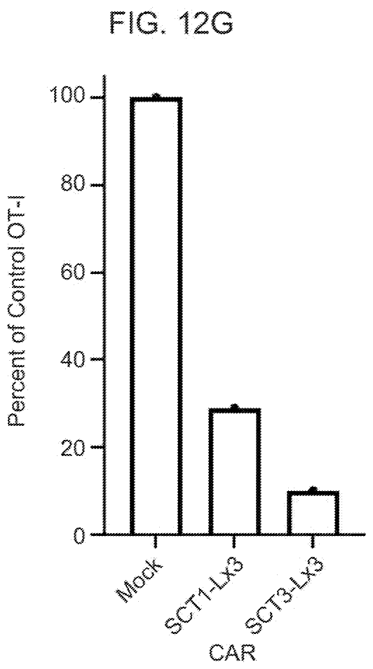

Mutation to the CD8 binding site confers additional survival advantage to bait CAR T cells over OT-I cells compared to mock T cells. The CD8-null binding mutant SCT3 comprises a Q226L and D227N mutation in the MHCI domain. FIG. 12A shows that when incubated with mock T cells, about 35.6% of OT-I cells survived, compared to about 9.72% when incubated with the first CD8 binding mutant SCT2 (FIG. 12B), and about 7.47% when incubated with the CD8-null binding SCT3 variant (FIG. 12C). FIG. 12D and FIG. 12E show that bait CAR T cells with CD8 null binder (SCT3) decreases the activation in OT-I cells. FIG. 12F and FIG. 12G show that bait CAR T cells with CD8 null binder (SCT3) exhibits increased targeted killing of OT-I cells than wildtype MHCI domain (SCT1).

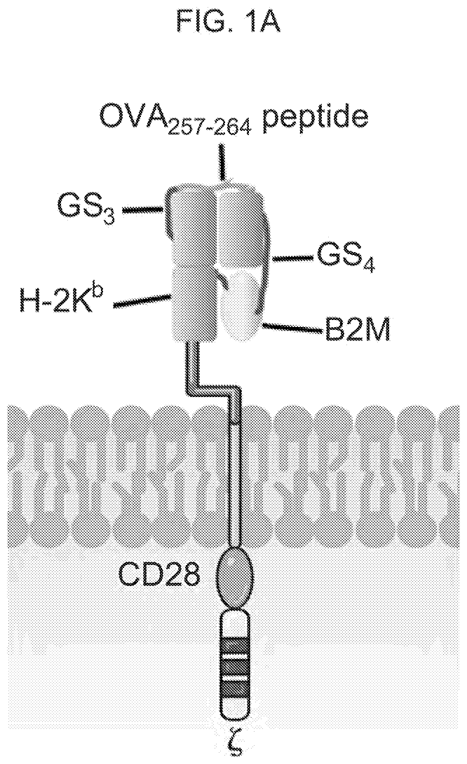

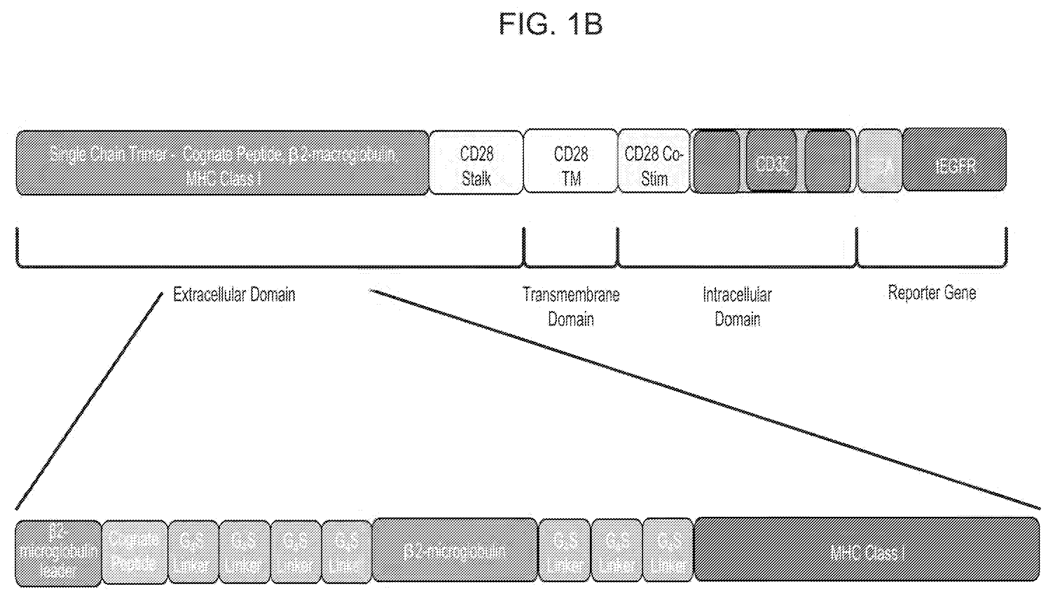

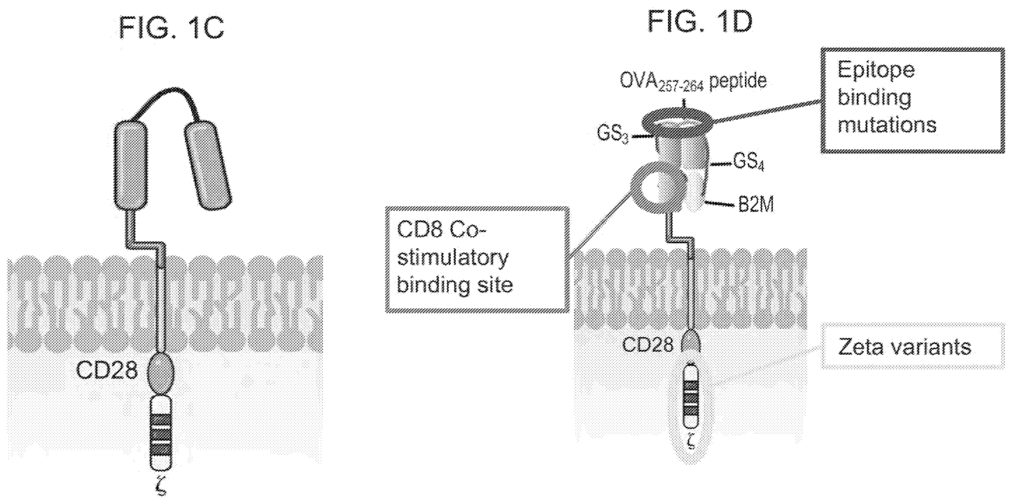

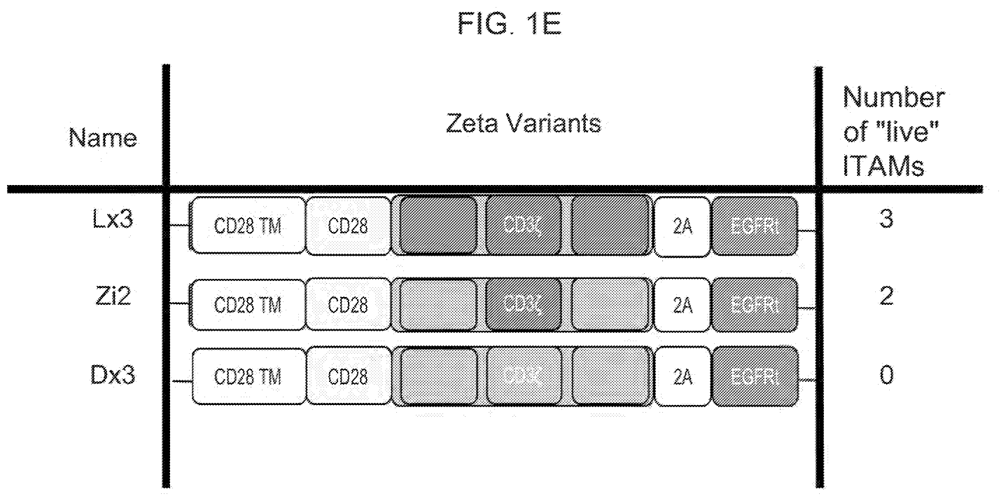

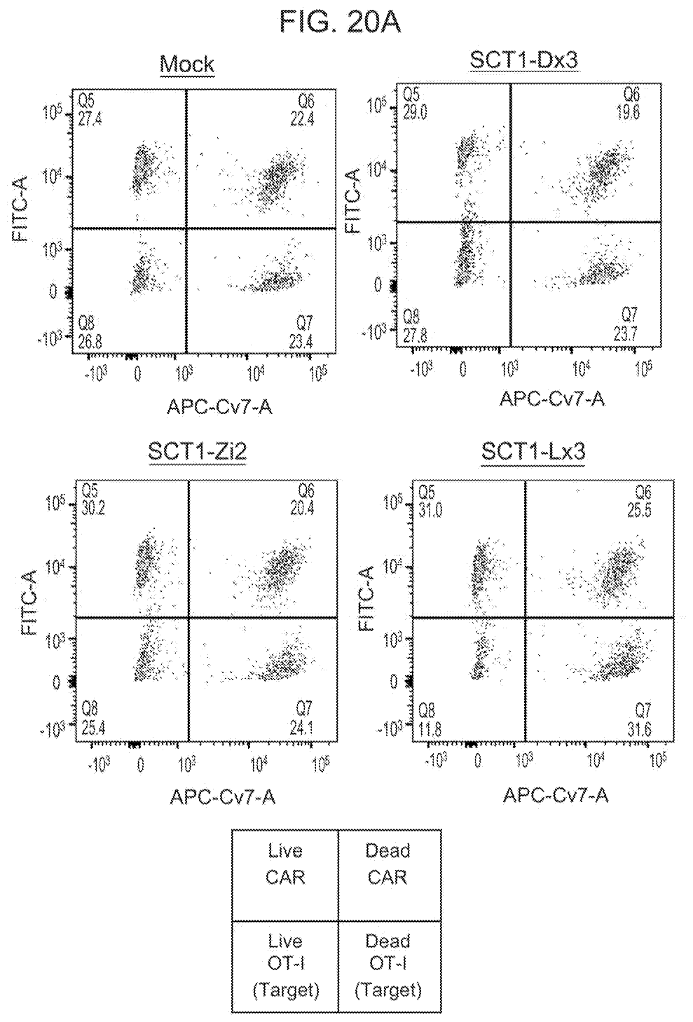

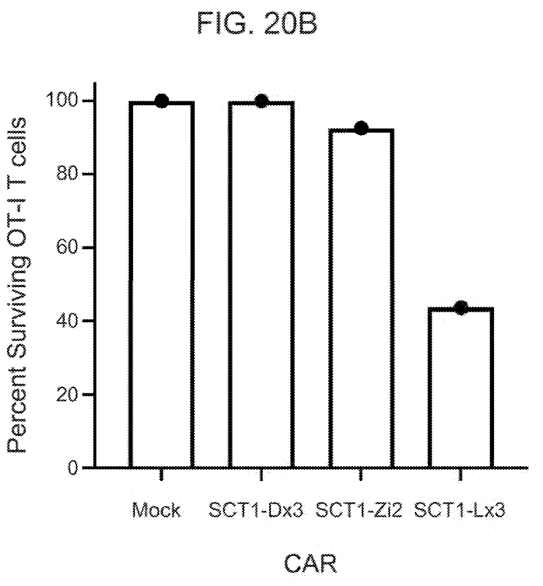

Mutations of CD3 zeta intracellular domains (zeta variants) were designed to maximize the number of live ITAM domains to improve signaling. Schematics of zeta variants are shown in FIG. 1D and FIG. 1E. These zeta variants confer higher intracellular signaling and functional killing of target cells by bait CAR T cells. FIG. 20A is a series of flow cytometry plots showing that maximizing the number of live ITAM domains in the CD3 zeta intracellular domain of the bait CAR T cells increases in vitro killing of OT 1 cells. Mock cells. SCT-1-Dx3 CAR T cells. SCT1-Zi2 CAR T cells, and SCTI-Lx3 CAR T cells were tested. FIG. 20B is a graph showing the quantification of the results shown in FIG. 20A. The y axis shows the percent of surviving OT-I cells following contact with Mock cells. SCT-1-Dx3 CAR T cells. SCT1-Zi2 CAR T cells, and SCT1-Lx3 CAR T cells.

### Example 5: MHC-I Bait CAR In Vivo Profile Vs Polyclonal Target T Cells

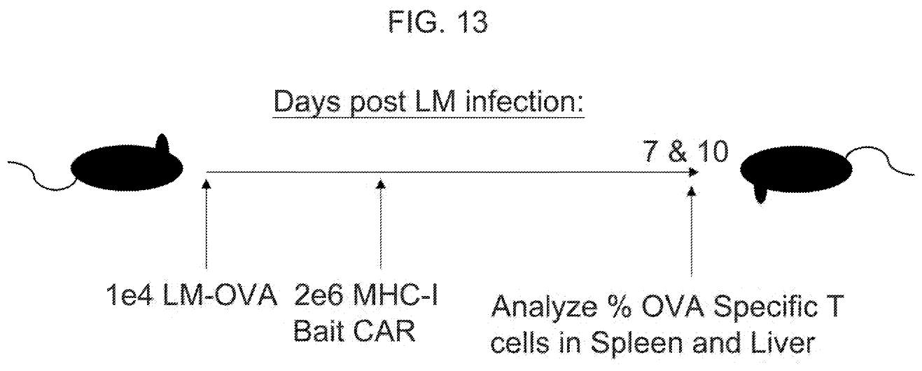

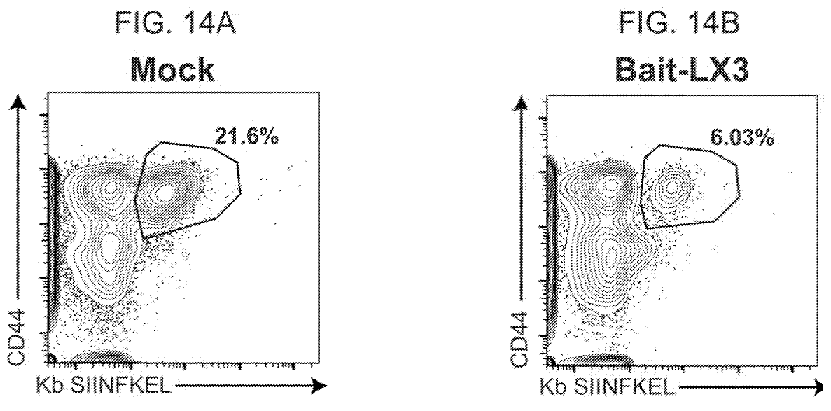

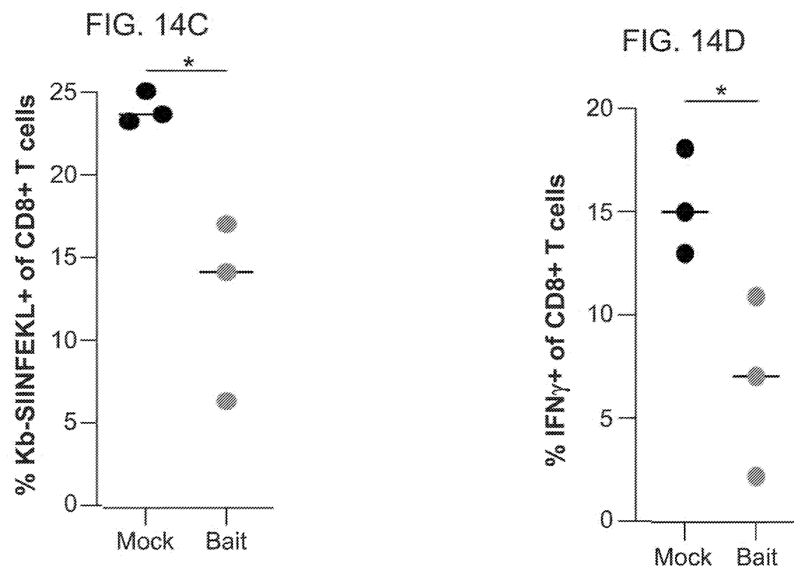

The depletion of targeted effector T cells by exemplary MHC-1 bait CAR T cells in vivo was shown using a first model, depicting in FIG. 13. The percentage of target effective (OVA) T cells in the spleen and liver of mice was quantified after 7-10 days. A lower number of OVA-specific effector T cells (about 6.03%) was detected in the tissue of animals treated with exemplary MHC-1 bait CAR T cells, as shown in FIG. 14B, compared to animals treated with mock CAR T cells, which contained about 21.6% OVA-specific T cells (FIG. 14A). FIG. 14C shows that the number of peptide-target containing effector CD8 T cells was lower (˜14%) following treatment with exemplary bait CAR T cells when compared to the number of peptide-target containing effector CD8 T cells following mock CAR T-treatment (˜24%). FIG. 14D shows that the number of SIINFEKL-tetramer stimulated CD8 T cells which produce IFN-gamma was lower following treatment with exemplary bait CAR T cells (˜7%) compared with treatment with mock CAR T cells (˜15%).

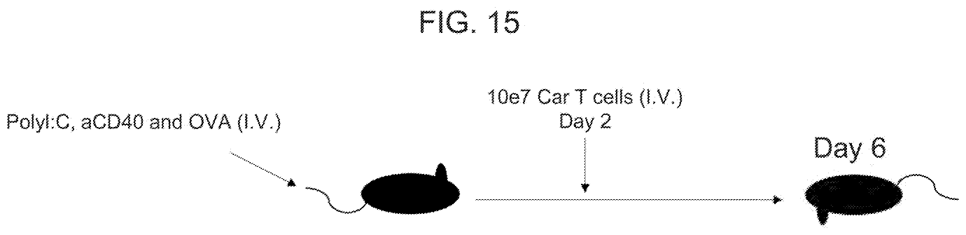

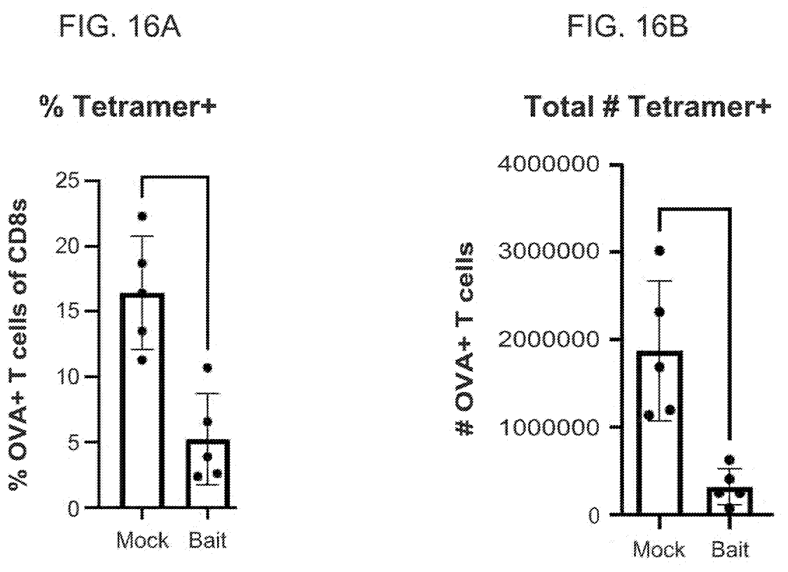

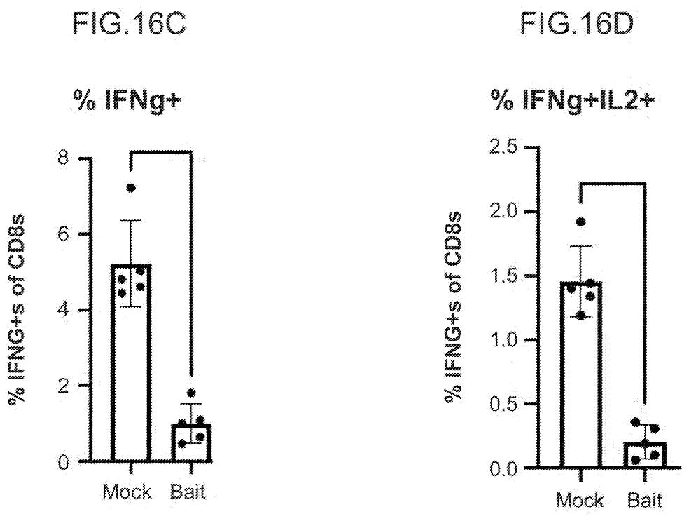

FIG. 15 shows a second model was used to show the depletion of targeted effector T cells by exemplary MHC-1 bait CAR T cells in vivo. In the exemplary bait CAR T cell-treated animals, the percentage of target peptide-positive CD8 T cells (FIG. 16A), total number of target peptide-reactiveT cells (FIG. 16B), percentage of IFN-gamma producing CD8 T cells after stimulation (FIG. 16C) and total number of IFN-gamma positive. IL-2 producing CD8 T cells after stimulation (FIG. 16D) were lower than in mock-treated animals.

### Example 6: Killing of T Cells Via Targeting of T Cell Receptors Using Two Novel Chimeric Antigen Receptor Designs

In Vitro Co-Culture Results in Reciprocal Activation of Both CAR T Cells and Target OT-I T cells CAR T cell and OT-I T cell in vitro co-culture experiments.

Two novel CAR designs which target T cells via their T cell receptor (TCR) were tested. OT-I T cells, a model system with a known antigenic epitope. Bait CARs tested were the anti-Vβ5 bait CAR, a second-generation CAR with a traditional extracellular scFv domain that targets Vβ5 of the OT-I TCR (FIG. 1C) and the H2-K^{b} Bait CAR, which instead has an extracellular H2-K^{b} complex loaded with the SIINFEKL cognate epitope.

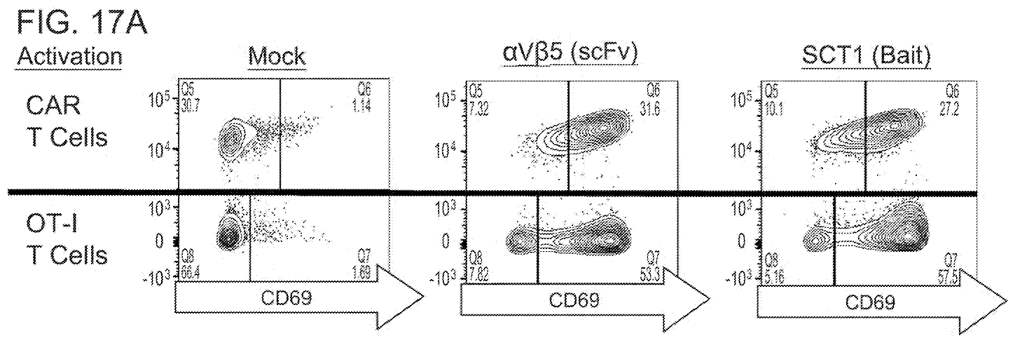

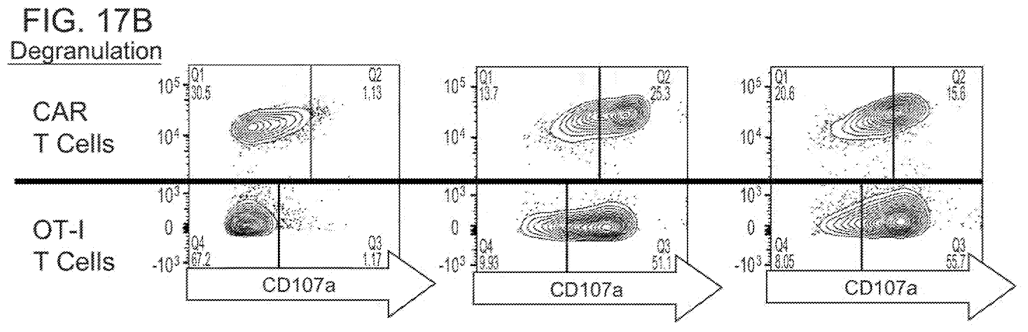

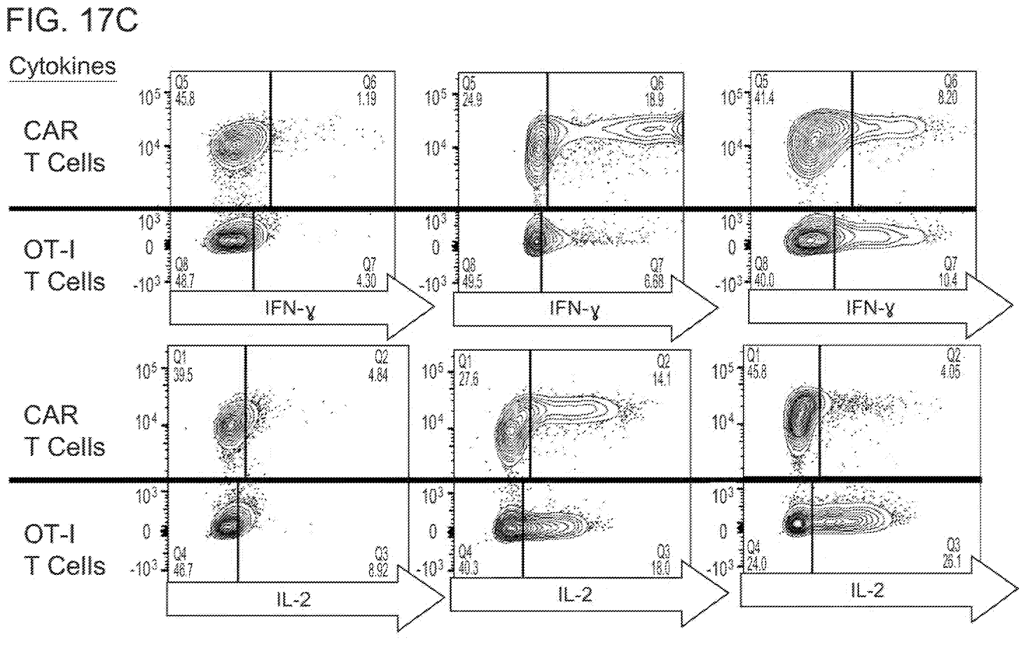

Mock, anti-Vβ5 and OVA-MHC-I Bait CAR (SCT1) were co-incubated with purified, naïve OT-I T cells for 12 hours, then analyzed by flow cytometry as both cell populations are T cells capable of making these markers. Results are shown in FIG. 17A-17C. Activation displayed by CD69 staining (FIG. 17B) and degranulation (CD107a: FIG. 17B), as well as production of cytokines IFN-γ and IL-2 (FIG. 17C). As expected, both CAR T cells and target OT-I T cells showed robust activation by most metrics.

### In Vitro, αVβ5 CAR T Cells Kill OT-I T Cells More Quickly, Likely Due to More Rapid Activation and More Cytokine Production

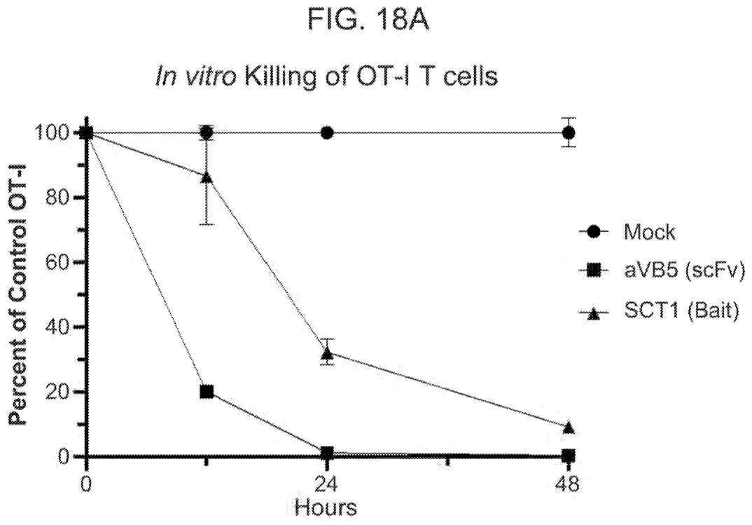

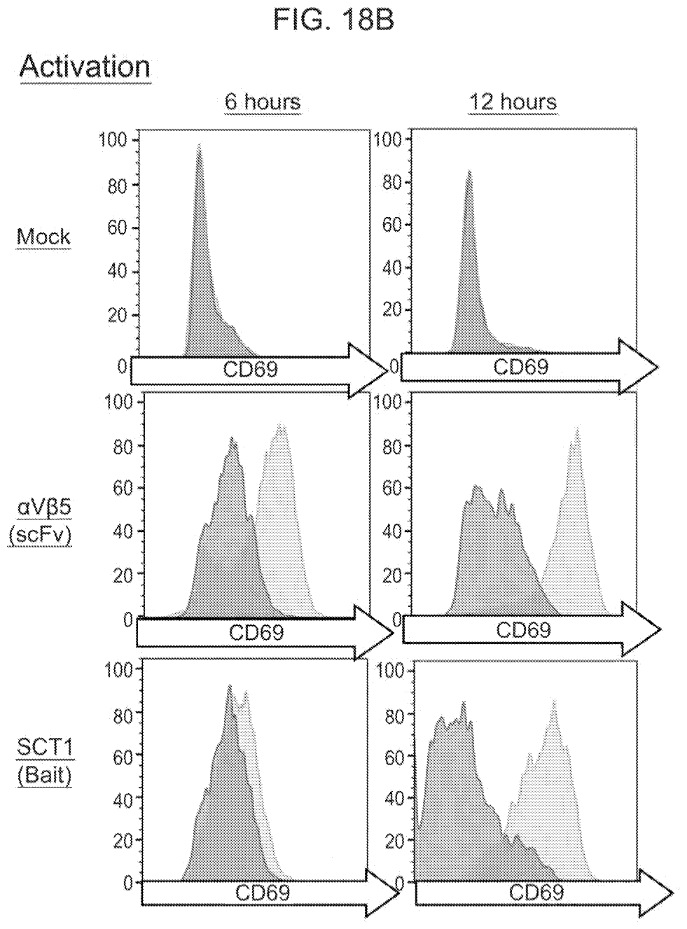

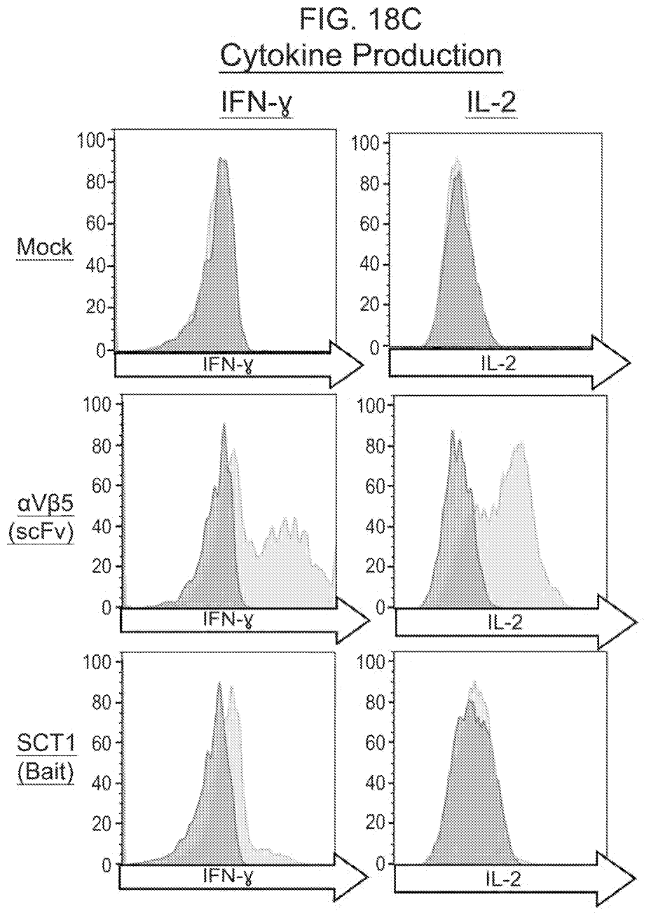

CAR T cells and OT-I were co-cultured over 48 hrs with cytokines, and the percentage of remaining OT-I T cells was measured at different timepoints, αVβ5 CAR T cells proved to kill much quicker than OVA-MHC-I Bait CAR T cells (FIG. 18A). This appears to be due to delayed (though not incomplete) activation of H2-K^{b} Bait CAR (FIG. 18B) as well as little to no cytokine production (FIG. 18C).

In Vivo, OVA-MHC-I Bait CAR T Cells are More Effective at Depleting Polyclonal OVA-Reactive T Cells Testing CAR T Cells Using In Vivo Models with Polyclonal OVA-Reactive T Cells.

Adjuvanted ovalbumin vaccine or ovalbumin-expressing Listeria monocytogenes (LM-OVA) given to wild type mice will generate a robust, repeatable T cell response to full length ovalbumin. Mock or [OVA-MHC-I (Bait)] CAR T cells given to these mice over the following days demonstrate the ability of the CAR T cells to eliminate the ovalbumin-reactive T cells, as measured by SIINFEKL-H-2K^{b} tetramer staining. To test if reduction in the targeted ovalbumin-reactive T cells was functionally relevant, adjuvanted ovalbumin vaccine receiving mice were given mock or [OVA-MHC-I (Bait)] CAR T cells and then challenged with LM-OVA infection 3 weeks later. Mice which had lower number of target ovalbumin-reactive T cells will show less ability to suppress or fight the LM-OVA infection and have more of these bacteria grow out of their livers.

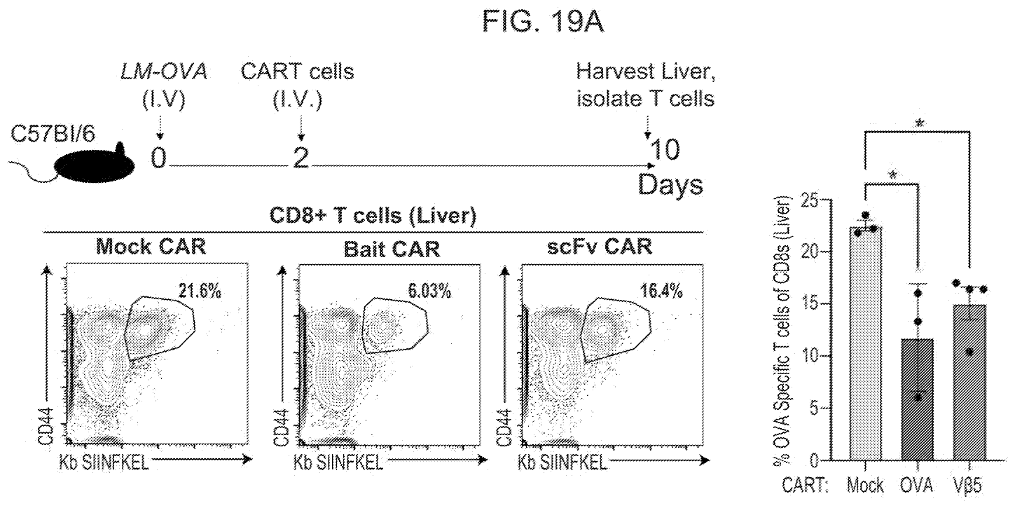

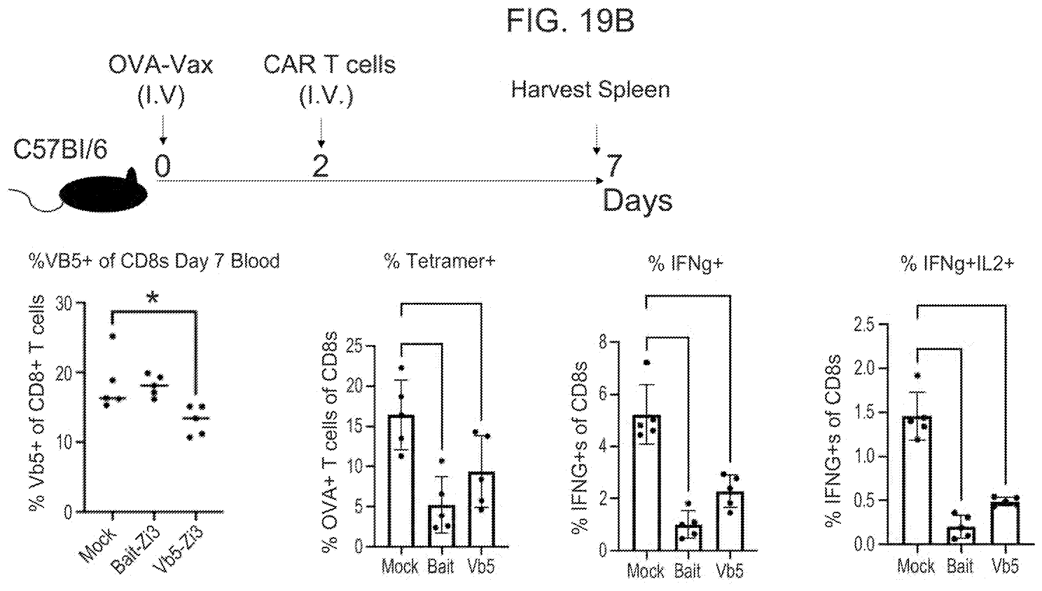

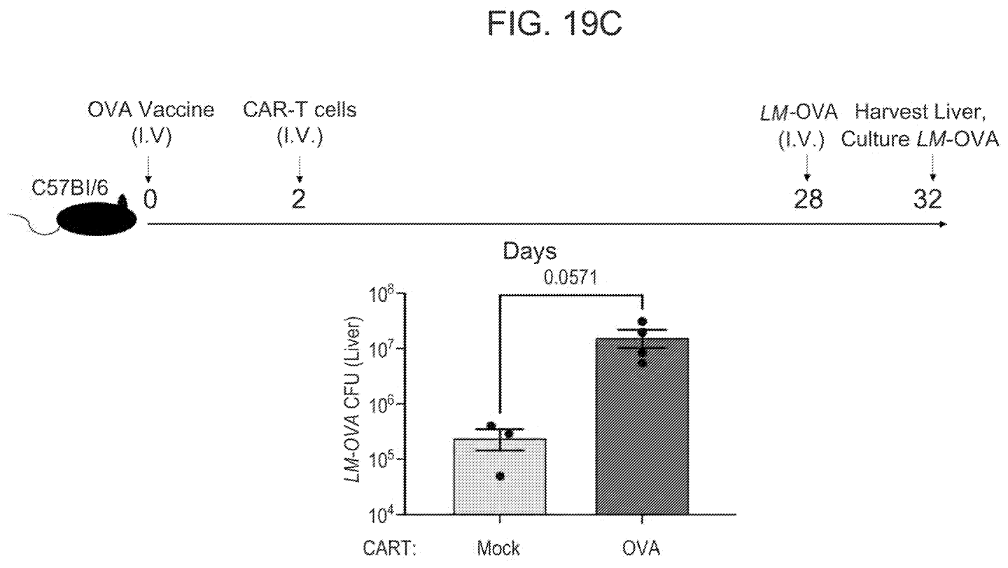

Mice were given OVA-expressing Listeria monocytogenes to generate a robust OVA-reactive effector T cell response, then given CAR T cells 2 days later. The OVA-MHC-I Bait CAR was able to deplete more target cells (FIG. 19A). A polyclonal effector T cell response was generated with OVA vaccination plus adjuvant, and again the OVA-MHC Class I CAR T cells did better than the scFv CAR T cells at depleting OVA-reactive cells, including tetramer-induced IFNγ and IFNγ/IL-2 positive cells (FIG. 19B). To test the biological/functional relevance of this finding. OVA-vaccinated mice were challenged 28 days later with LM-OVA infection. Mice that received OVA-MHC Class I Bait CARs had more severe infection (FIG. 19C).

### CONCLUSIONS

Two CAR systems for the targeting of T cells via their TCR were developed. While the more classical, scFv CAR appears to work more robustly in vitro, in vivo experiments with multiple models of polyclonal OVA-reactive T cells demonstrate that the OVA-MHC Class I Bait CAR was superior at depleting OVA-reactive T cells. This lack of correlation between in vitro and in vivo results could be due to differing number of target cells needed to be killed, but it should be noted that this is frequently seen with CAR T cells.

### Example 7: Effects of OVA-MHC-I Bait CAR T Cells on a Mouse Model of Type 1 Diabetes

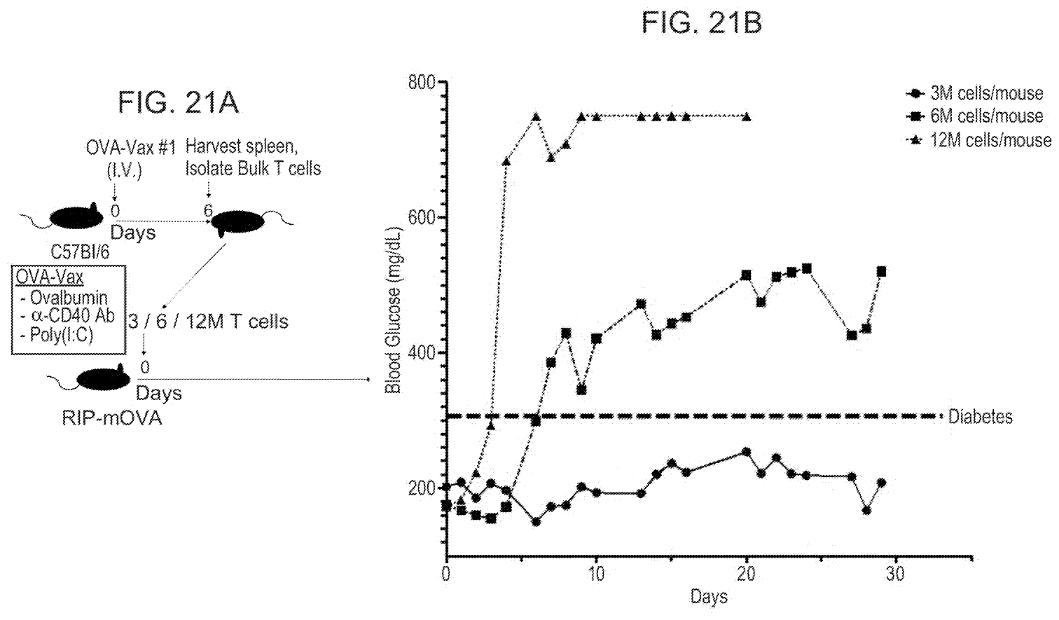

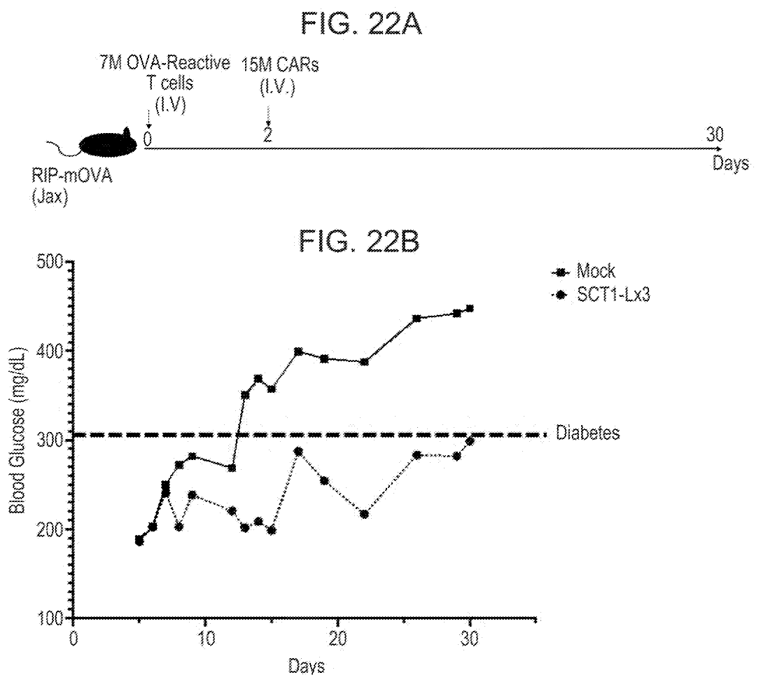

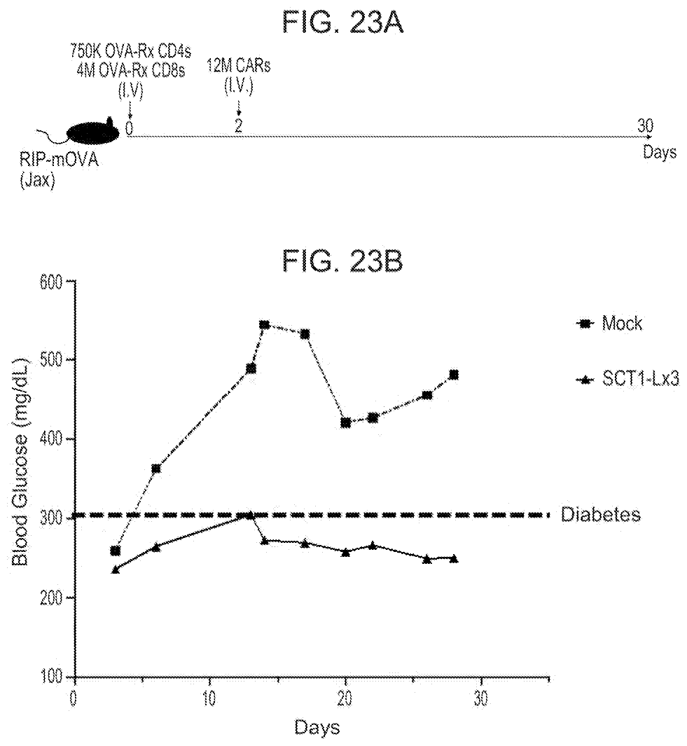

Type 1 diabetes (TID) was induced in RIP-mOVA mice via adoptive transfer of polyclonal. OVA-reactive T cells (FIG. 21A and FIG. 21B). These OVA-reactive T cells were induced in wild type mice via adjuvanted vaccination with full length ovalbumin, then isolated from the spleens of these mice either as bulk T cells (FIG. 22A and FIG. 22B) or via separate isolations of CD4 and CD8 T cells (FIG. 23A and FIG. 23B). Adoptive transfer of these OVA-reactive T cells into RIP-mOVA mice induce hyperglycemia and diabetes within 7-14 days as measured by blood glucose. Mock and [OVA-MHC-I (Bait)] CAR T cells are given 2 days later and the mice are monitored to determine if Type 1 diabetes is prevented. Altogether, the results demonstrate that the OVA-MHC-I Bait CAR T cells reduce blood glucose levels and prevent the occurrence of Type 1 diabetes over a prolonged period of time (up to 30 days).

### Example 8: In Vitro Effects of Human HLA-A2-NYESO Bait CAR

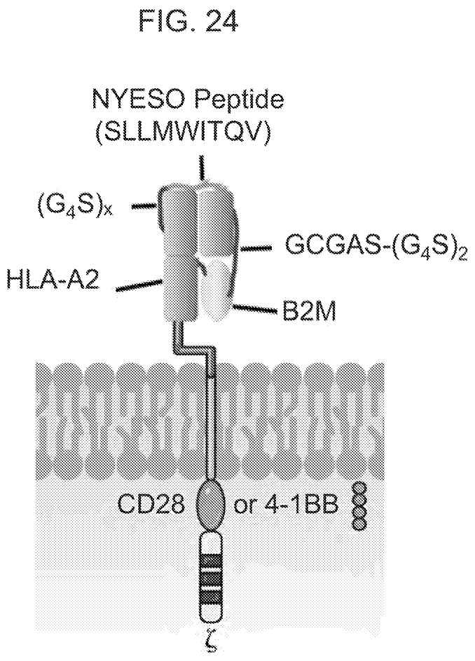

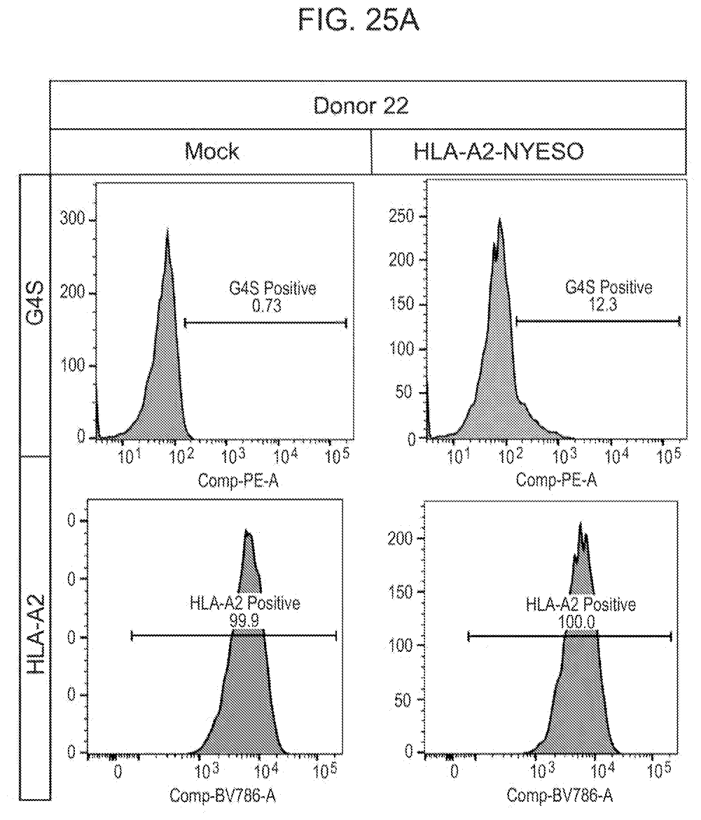

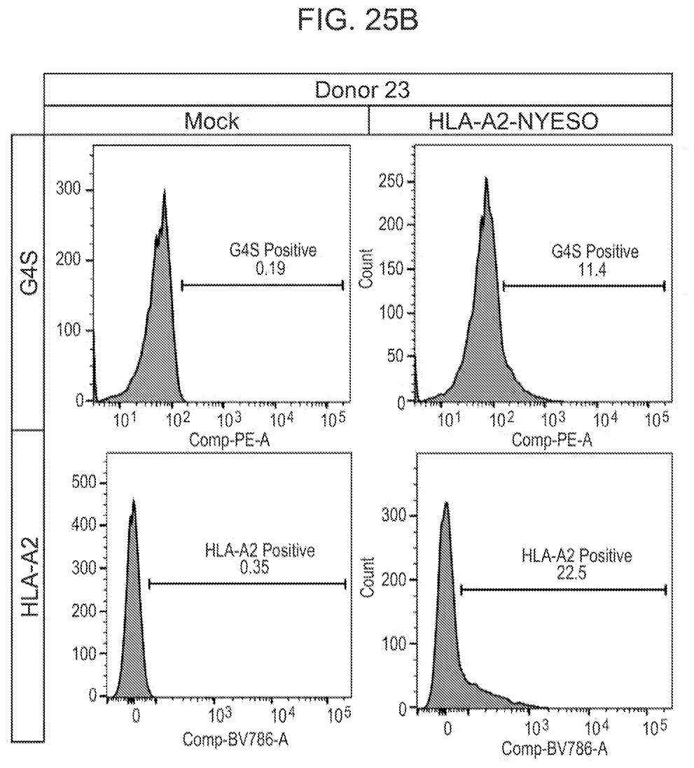

A HLA-A2-NYESO bait CAR (also known as NYESO-SCT_28z bait CAR) (FIG. 24) or Mock transduced human T-cells and donor matched m1G4 TCR transduced human T-cells were cocultured at a 1:1 ratio in duplicate wells. Replicate plates were made to allow for multiple assays and timepoints. FIG. 25A and FIG. 25B show the transduction efficiency and expression of the HLA-A2-NYESO bait CAR on T cells.

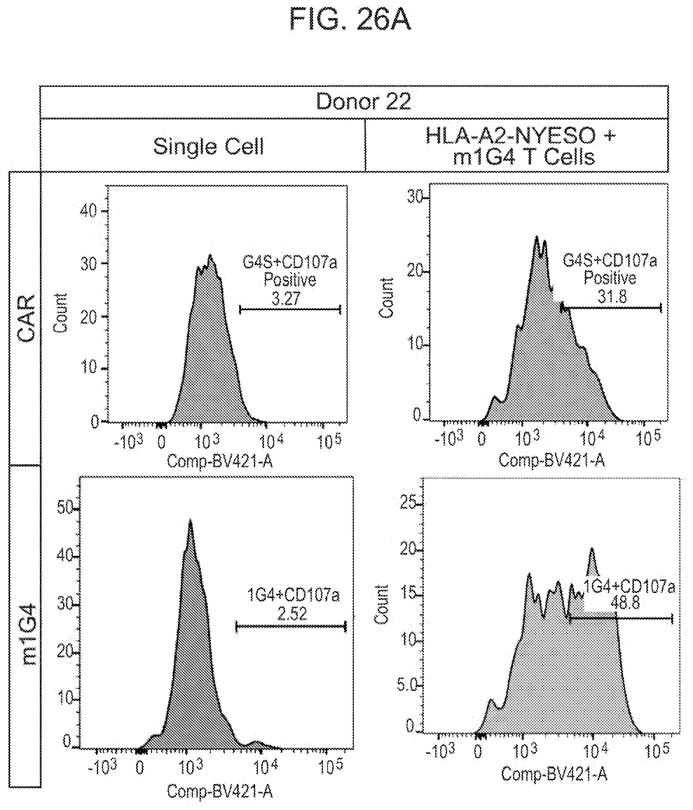

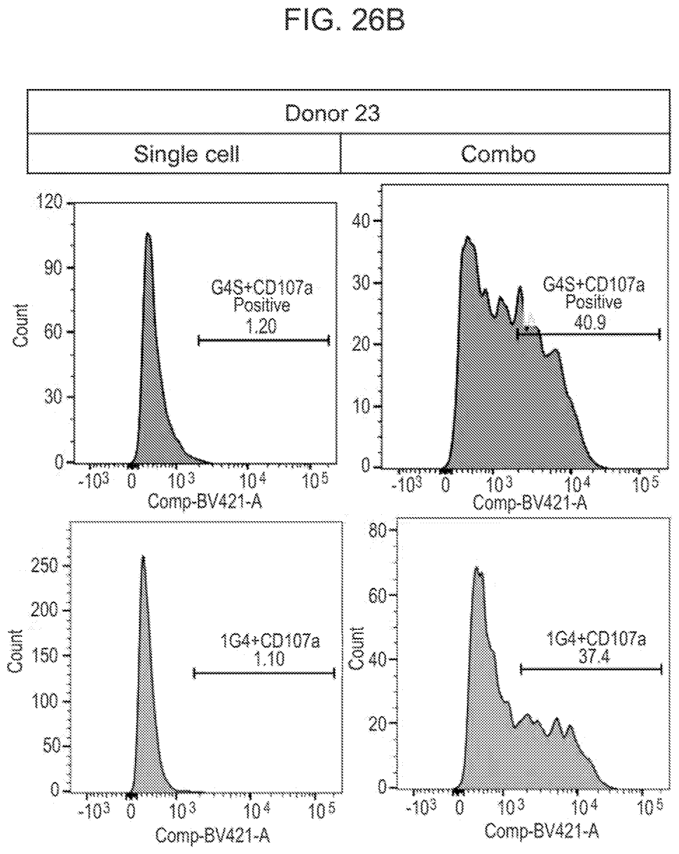

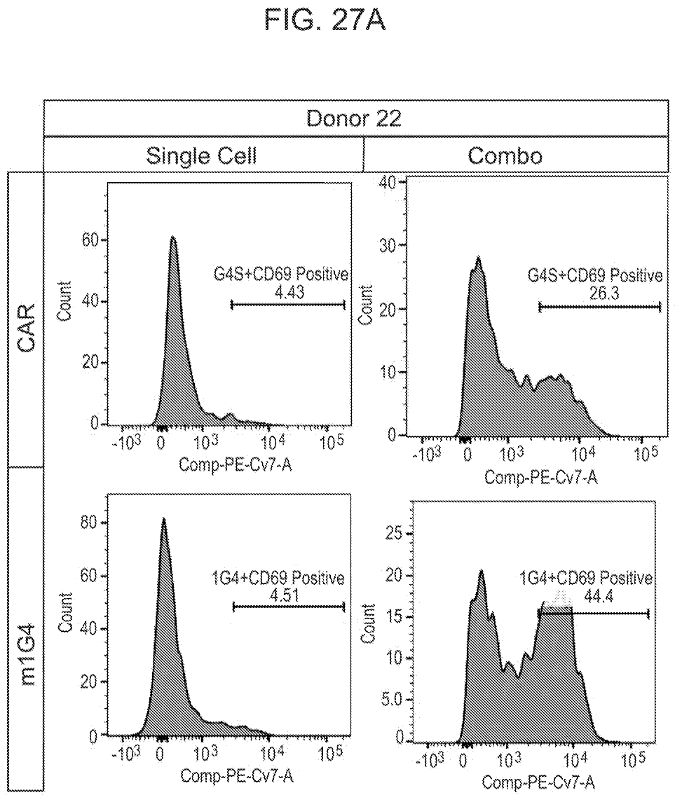

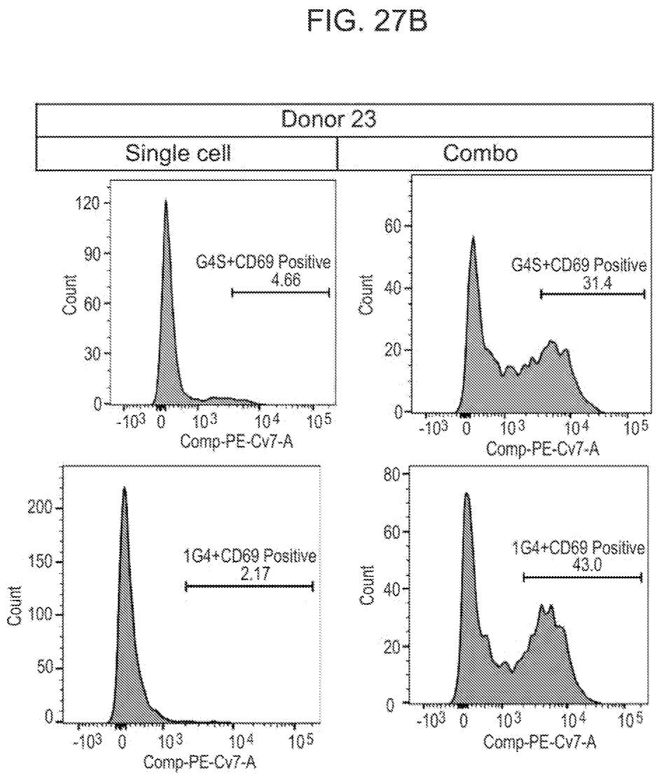

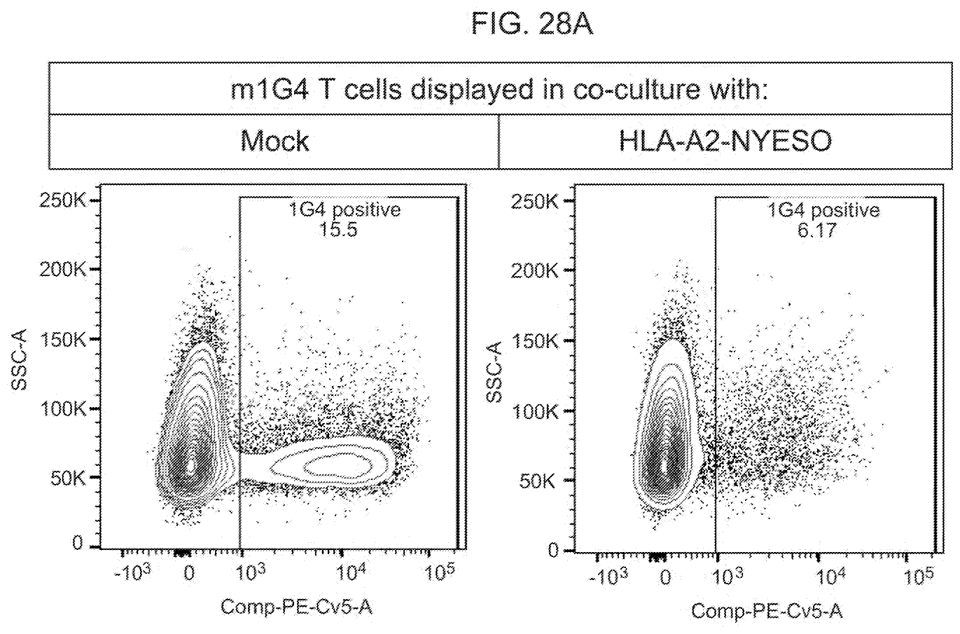

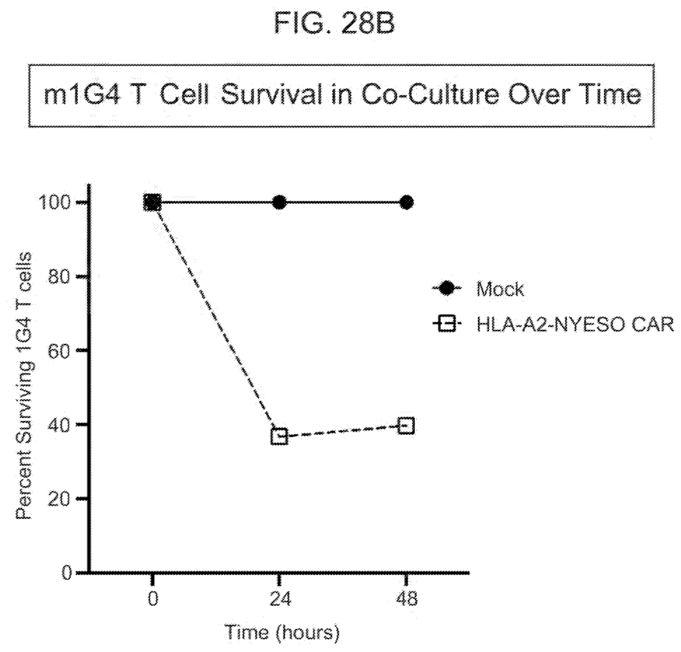

After 6 hrs of coculture one plate was pulled to assess early activation markers by flow cytometry (CD107a &CD69) on both the NYESO-SCT_28z bait CAR and the m1G4 TCR T-cells. Representative histograms of duplicate coculture wells are shown for CD107a as a proxy for degranulation (FIG. 26A and FIG. 26B) and for CD69 as a proxy for activation (FIG. 27A and FIG. 27B). Killing was assessed after 24 and 48 hrs of coculture by flow cytometry (FIG. 28A). The ratio of m1G4 TCR to NYESO-SCT_28z bait CAR was normalized to the appropriate Mock samples and data is represented as percent survival of m1G4 TCR T-cells (FIG. 28B).

### Example 9: In Vivo Effects of Human HLA-A2-NYESO Bait CAR

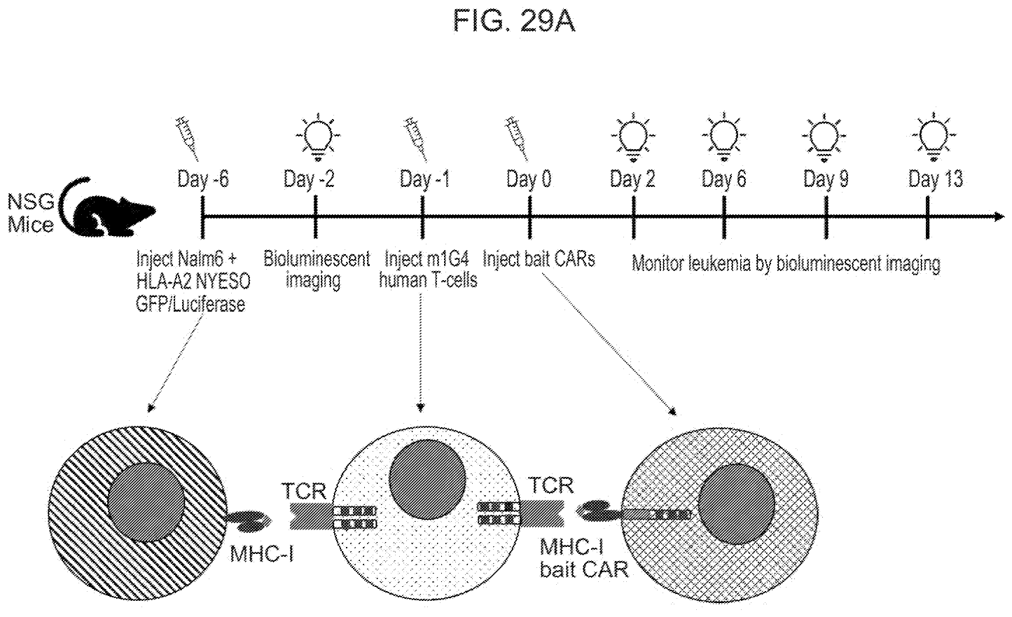

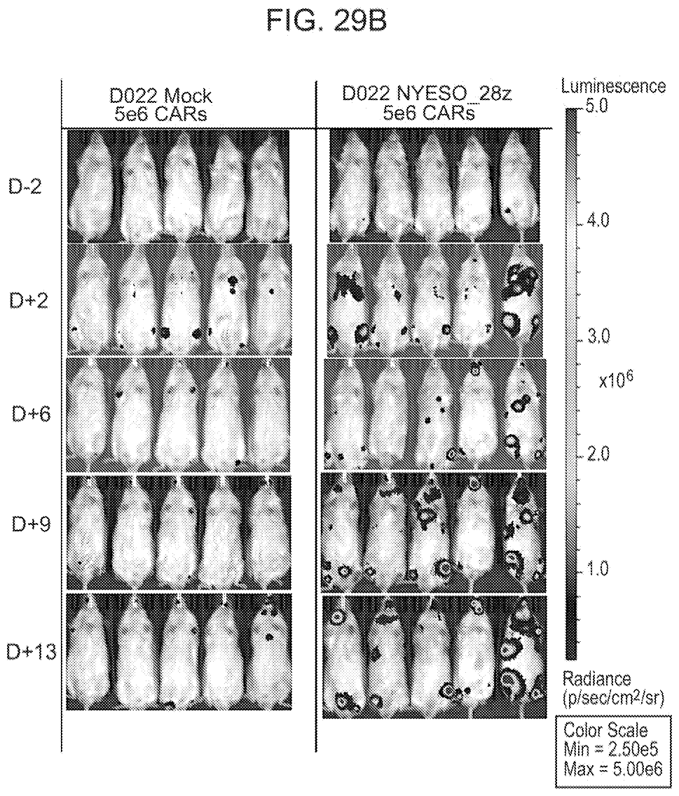

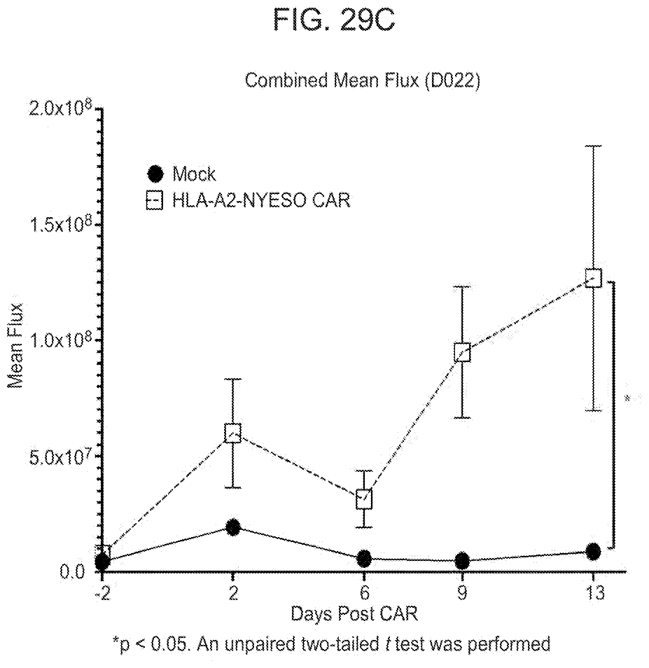

An in vivo xenograft experiment in NSG mice to test the NYESO-SCT_28z bait CAR against m1G4 TCR transduced T-cells (FIG. 29A). Both the CAR and TCR T cell populations were donor matched to prevent alloreactivity. A Nalm6 variant that expresses the NYESO single chain trimer as well as GFP and firefly luciferase was used. This cell line will act as a target for the m1G4 TCR T cells. The m1G4 TCR+ cells should clear or at least slow the leukemia progression on their own. If the number of m1G4 TCR+ cells in each mouse with the NYESO Bait CARs is significantly reduced, the pressure on the leukemia should be removed allowing the leukemia to grow out. Therefore, the recurrence of leukemia following the administration of the Bait CAR is a proxy for the functional and targeted killing of m1G4 TCR+ cells by the Bait CAR.

On D-6 NSG mice were injected with 1 million Nalm6 G/L+NYESO_SCT leukemia cells via the tail vein. On D-2 mice were checked for leukemia engraftment by bioluminescent imaging. On D-1 mice were injected with 500,000 m1G4 TCR transduced human T-cells via the tail vein. On D=0 mice were injected with 4 million donor matched, NYESO-SCT_28% bait CAR transduced human T-cells via the tail vein. Leukemia progression was assessed by bioluminescent imaging on D+2, D+6, D+9, & D+13 on an IVIS Spectrum (FIG. 29B). While body BLI images and mean luminescence (Flux) are shown (FIG. 29C). Together these results show the targeted killing of m1G4 TCR+ cells by the HLA-A2-NYESO Bait CAR.

### IX. Embodiments

Embodiment 1. A chimeric antigen receptor (CAR) comprising:
(a) an ectodomain comprising
   i. a beta-2 microglobulin leader peptide (B2ML),
   ii. a cognate peptide that is recognized by a CD8+ T-cell Receptor (P),
   iii. at least one linker domain (L),
   iv. a beta-2 microglobulin peptide (B2M), and
   v. a MHC class I (MHCI), a HLA-A, a HLA-B or a HLA-C:
(b) a transmembrane domain;
(c) at least one costimulatory domain; and
(d) an intracellular signaling domain.

Embodiment 2. The CAR of Embodiment 1, wherein the ectodomain comprises the following in the N-terminal to C-terminal direction:
N-term-B2ML-P-(L)ₓ-B2M-(L)_{y}-(MHCI/HLA-A/HLA-B/HLA-C)-C-term
   wherein x is any integer between 0-5; and
   wherein y is any integer between 0-5.

Embodiment 3. The CAR of any one of embodiments 1-2, wherein the cognate peptide is isolated or derived from ovalbumin or an autoantigen of a autoimmune disease.

Embodiment 4. The CAR of any one of embodiments 1-2, wherein the cognate peptide comprises the amino acid sequence SX₁X₂X₃FEKL (SEQ ID NO: 62),
wherein
X₁ is A or I:
X₂ is I or Y; and
X₃ is N, Q, T or V.

Embodiment 5. The CAR of any one of embodiments 4, wherein the cognate peptide comprises the amino acid sequence of SEQ ID NO: 7-10, or 56.

Embodiment 6. The CAR of any one of embodiments 1-5, wherein the B2ML is a mouse B2ML or a human B2ML.

Embodiment 7. The CAR of any one of embodiments 1-6, wherein the B2ML comprises the amino acid sequence of SEQ ID NO: 1.

Embodiment 8. The CAR of any one of embodiments 1-7, wherein the at least one linker domain comprises the amino acid sequence of SEQ ID NO: 11, 57 or 58.

Embodiment 9. The CAR of any one of embodiments 1-8, wherein the B2M is a mouse B2M or a human B2M.

Embodiment 10. The CAR of any one of embodiments 1-9, wherein the B2M comprises the amino acid sequence of SEQ ID NO: 2.

Embodiment 11. The CAR of any one of embodiments 1-10, wherein the ectodomain of (a) comprises a MHCI.

Embodiment 12. The CAR of any one of embodiment 11, wherein the MHCI comprises the amino acid sequence of SEQ ID NO: 3.

Embodiment 13. The CAR of embodiment 11, wherein the MHCI comprises a mutation in the epitope binding domain of the MHCI.

Embodiment 14. The CAR of any one of embodiments 11 or 13, wherein the MHCI comprises a mutation in the CD8 binding site domain of the MHCI.

Embodiment 15. The CAR of any one of embodiments 13-14, wherein the MHCI comprises the amino acid sequence of SEQ ID NO: 3-5.

Embodiment 16. The CAR of any one of embodiments 1-10, wherein the ectodomain of (a) comprises a HLA-A, a HLA-B or a HLA-C.

Embodiment 17. The CAR of any one of embodiments 1-16, wherein the transmembrane domain comprises a CD28, CD8, CD8a, CD8 beta, CD3-epsilon, CD3-delta, CD3-gamma, CD3z, CD4, 4-1BB, OX40, ICOS, PD-1, LAG-3, 2B4 or BTLA transmembrane domain or a portion thereof.

Embodiment 18. The CAR of embodiment 17, wherein the transmembrane domain comprises a CD28 transmembrane domain.

Embodiment 19. The CAR of embodiment 18, wherein the CD28 transmembrane domain comprises the amino acid sequence of SEQ ID NO: 12.

Embodiment 20. The CAR of any one of embodiments 1-19, wherein the at least one costimulatory domain comprises a CD28, 4-1BB (CD137), CD97, CD11a-CD18, CD2, ICOS, CD27, CD154, CD8a, OX40 (CD134) co-stimulatory domain or a portion thereof.

Embodiment 21. The CAR of embodiment 20, wherein the at least one costimulatory domain comprises a CD28 costimulatory domain.

Embodiment 22. The CAR of embodiment 21, wherein the CD28 costimulatory domain comprises the amino acid sequence of SEQ ID NO: 14.

Embodiment 23. The CAR of any one of embodiments 1-22, wherein the intracellular signaling domain comprises a CD3ξ intracellular signaling domain.

Embodiment 24. The CAR of embodiment 23, wherein the CD3ξ intracellular signaling domain comprises a mutation in at least one of the ITAM domains of the CD35 intracellular signaling domain.

Embodiment 25. The CAR of any one of embodiments 23-24, wherein the CD3ξ intracellular signaling domain comprises the amino acid sequence of SEQ ID NO: 16-19.

Embodiment 26. The CAR of embodiment 1, wherein the CAR comprises: (a) an ectodomain comprising
i. a beta-2 microglobulin leader peptide (B2ML) comprising the amino acid sequence of SEQ ID NO: 1,
ii. a cognate peptide that is recognized by a CD8+ T-cell Receptor (P) comprising the amino acid sequence of SEQ ID NO: 6, 7, 8, 9, 10, 56 or 62,
iii. at least one linker domain (L) comprising the amino acid sequence of SEQ ID NO: 11, 57 or 58,
iv. a beta-2 microglobulin peptide (B2M) comprising the amino acid sequence of SEQ ID NO: 2, and
v. (a) MHC class I (MHCI) comprising the amino acid sequence of SEQ ID NOs: 3-5:
(b) a transmembrane domain comprising the amino acid sequence of SEQ ID NO: 12:
(c) a costimulatory domain comprising the amino acid sequence of SEQ ID NO: 14; and
(d) a intracellular signaling domain comprising the amino acid sequence of SEQ ID NO: 18 or 19.

Embodiment 27. The CAR of embodiment 26, wherein the CAR comprises the amino acid sequence of SEQ ID NOs: 20, 22, 24, 26, 28, 30 or 32.

Embodiment 28. A polynucleotide comprising a nucleic acid encoding the CAR of any one of embodiments 1-27.

Embodiment 29. A vector comprising the polynucleotide of embodiment 28.

Embodiment 30. An immune cell expressing the CAR of any one of embodiments 1-27.

Embodiment 31. The immune cell of embodiment 30, wherein the immune cell is a T-cell, a hematopoietic progenitor cell, a peripheral blood (PB) derived T-cell or an umbilical cord blood (UCB) derived T-cell.

Embodiment 32. The immune cell of embodiment 30, wherein the immune cell is a CD8+ T-cell.

Embodiment 33. A composition comprising the immune cell of any one of embodiments 30-32 and a pharmaceutically acceptable carrier.

Embodiment 34. A method of targeting a CD8+ T-cell comprising providing a population of the immune cells of any one of embodiments 30-32.

Embodiment 35. A method of treating a condition associated with pathologic T cells in a subject, comprising administering to a subject in need thereof an effective amount of the composition of embodiment 33.

Embodiment 36. The method of embodiment 35, wherein the condition is an autoimmune disease, a transplant rejection, or a chronic inflammatory disease.

1. A cell comprising a chimeric antigen receptor (CAR) comprising:
(a) an ectodomain comprising:
(i) a beta-2 microglobulin leader peptide (B2ML),
(ii) a cognate peptide that is recognized by a CD8+ T-cell Receptor (P),
(iii) at least one linker domain,
(iv) a beta-2 microglobulin peptide (B2M),
(v) a MHCI, a HLA-A, a HLA-B or a HLA-C, and
(vi) a stalk/hinge domain
(b) a transmembrane domain;
(c) at least one costimulatory domain; and
(d) an intracellular signaling domain.

2. The cell of claim 1, wherein the ectodomain comprises the following in the N-terminal to C-terminal direction:
N-term-B2ML-P-(Linker 1)ₓ-(Linker 2)_{y}-B2M-(Linker 2)_{z}-(MHC-I/HLA-A/HLA-B/HLA-C)-stalk/hinge-C-term
wherein x is any integer between 0-5;
wherein y is any integer between 0-5; and
wherein z is any integer between 0-5.

3. The cell of claim 1, wherein the cognate peptide is isolated or derived from an antigen of an autoimmune disease or a chronic inflammatory disease.

4. The cell of claim 1, wherein the CAR comprises:
(a) an ectodomain comprising:
(i) a human beta-2 microglobulin leader peptide (B2ML),
(ii) a cognate peptide that is recognized by a human CD8+ T-cell Receptor (P),
(iii) at least one linker domain,
(iv) a human beta-2 microglobulin peptide (B2M), and
(v) a HLA-A, a HLA-B or a HLA-C, and
(vi) a human stalk/hinge domain;
(b) a human transmembrane domain;
(c) at least one human costimulatory domain; and
(d) a human intracellular signaling domain.

5. The cell of claim 4, wherein the human B2ML comprises the amino acid sequence of SEQ ID NO: 73.

6. The cell of claim 4, wherein the at least one linker domain comprises the amino acid sequence of SEQ ID NO: 11, 57, 58, 70 or 86.

7. The cell of claim 4, wherein the human B2M comprises the amino acid sequence of SEQ ID NO: 75.

8. The cell of claim 4, wherein the ectodomain of (a) comprises an HLA-A comprising the amino acid sequence of SEQ ID NO: 76.

9. The cell of claim 4, wherein the ectodomain of (a) comprises at least one mutation in the CD8 binding domain of the HLA-A, HLA-B or HLA-C.

10. The cell of claim 4, wherein the ectodomain of (a) comprises an HLA-A, wherein the HLA-A comprises at least one mutation in the CD8 binding domain.

11. The cell of claim 10, wherein the HLA-A comprising at least one mutation in the CD8 binding domain comprises the amino acid sequence of SEQ ID NO: 77.

12. The cell of claim 1, wherein the cell is a T-cell, a hematopoietic progenitor cell, a peripheral blood (PB) derived T-cell or an umbilical cord blood (UCB) derived T-cell.

13. The cell of claim 12, wherein the cell is a CD8+ T-cell.

14. A composition comprising the cell of a cell comprising a chimeric antigen receptor (CAR) and a pharmaceutically acceptable carrier, wherein said CAR comprises:
(a) an ectodomain comprising:
(i) a beta-2 microglobulin leader peptide (B2ML),
(ii) a cognate peptide that is recognized by a CD8+ T-cell Receptor (P),
(iii) at least one linker domain,
(iv) a beta-2 microglobulin peptide (B2M),
(v) a MHCI, a HLA-A, a HLA-B or a HLA-C, and
(vi) a stalk/hinge domain
(b) a transmembrane domain;
(c) at least one costimulatory domain; and
(d) an intracellular signaling domain.

15. A pharmaceutical composition comprising:
i) a population of cells comprising about 1.0×10⁵ to about 1.0×10⁹ cells, wherein said cells comprise a chimeric antigen receptor (CAR) comprising:
(a) an ectodomain comprising:
(i) a beta-2 microglobulin leader peptide (B2ML),
(ii) a cognate peptide that is recognized by a CD8+ T-cell Receptor (P),
(iii) at least one linker domain,
(iv) a beta-2 microglobulin peptide (B2M),
(v) a MHCI, a HLA-A, a HLA-B or a HLA-C, and
(vi) a stalk/hinge domain
(b) a transmembrane domain;
(c) at least one costimulatory domain; and
(d) an intracellular signaling domain; and
ii) a pharmaceutically acceptable carrier,
wherein the pharmaceutical composition is suitable for administration to a human subject.

16. A method of inducing cell death of a population of CD8+pathologic T-cells in a human subject in need thereof, wherein said method comprises administering to the human subject a therapeutically effective amount of a pharmaceutical composition comprising:
i) a population of cells comprising about 1.0×10⁵ to about 1.0×10⁹ cells, wherein said cells comprise a chimeric antigen receptor (CAR) comprising:
(a) an ectodomain comprising:
(i) a beta-2 microglobulin leader peptide (B2ML),
(ii) a cognate peptide that is recognized by a CD8+ T-cell Receptor (P),
(iii) at least one linker domain,
(iv) a beta-2 microglobulin peptide (B2M),
(v) a MHCI, a HLA-A, a HLA-B or a HLA-C, and
(vi) a stalk/hinge domain
(b) a transmembrane domain;
(c) at least one costimulatory domain; and
(d) an intracellular signaling domain; and
ii) a pharmaceutically acceptable carrier,
wherein the pharmaceutical composition is suitable for administration to a human subject, and
wherein a plurality of said cells bind to a plurality of said CD8+pathologic T-cells in said human subject, thereby inducing cell death of said population of CD8+ pathologic T-cells in said human subject.

17. The method of claim 16, wherein said cell death of said population of CD8+ pathologic T-cells in said human subject is about 2-fold to about 100-fold higher than the cell death of a population of CD8+ pathologic T-cells in a human subject that has not been administered said pharmaceutical composition.

18. The method of claim 16, wherein said human subject has a condition selected from a group consisting of an autoimmune disease, a transplant rejection, and a chronic inflammatory disease.
